(19) United States
(12) Patent Application Publication
(10) Pub. No.: US 2009/0270394 A1
(43) Pub. Date: Oct. 29, 2009

(54) CYCLYLAMINE DERIVATIVES AS CALCIUM CHANNEL BLOCKERS

(76) Inventors: Robert Galemmo, JR., Vancouver (CA); Richard Holland, Vancouver (CA); Gabriel Hum, Langley (CA); Hossein Pajouhesh, Coquitlam (CA); Navjot Chahal, Vancouver (CA); Mehran Seid-Bagherzadeh, Vancouver (CA); Amy Girard, Burnaby (CA)

Correspondence Address: MORRISON & FOERSTER LLP, 12531 HIGH BLUFF DRIVE, SUITE 100, SAN DIEGO, CA, 92130-2040, US

(21) Appl. No.: 12/431,077

(22) Filed: Apr. 28, 2009

Related U.S. Application Data
(60) Provisional application No. 61/048,509, filed on Apr. 28, 2008.

(51) Int. Cl. A61K 31/5355; C07D 413/08; A61P 35/00

(52) U.S. Cl. 514/235.5; 544/124

(57) ABSTRACT

Methods and compounds effective in ameliorating conditions characterized by unwanted calcium channel activity, particularly unwanted N-type and/or T-type calcium channel activity are disclosed. Specifically, a series of compounds of substituted or unsubstituted cyclylamine derivatives as shown in formulas (1).

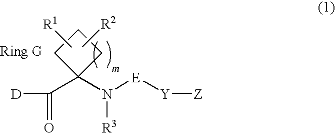

### RELATED APPLICATIONS

This application claims benefit of priority to U.S. Provisional Application Ser. No. 61/048,509 filed Apr. 28, 2008, the contents of which are incorporated herein by reference in its entirety.

### TECHNICAL FIELD

The invention relates to compounds useful in treating conditions associated with calcium channel function, and particularly conditions modulated by N-type and/or T-type calcium channel activity. More specifically, the invention concerns compounds containing substituted or unsubstituted cyclylamine derivatives that are useful in treatment of conditions such as pain, and other diseases or disorders of hyperexcitability such as cardiovascular disease and epilepsy.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Native calcium channels have been classified by their electrophysiological and pharmacological properties into T-, L-, N-, P/Q- and R-types (reviewed in Catterall, W., Annu Rev Cell Dev Biol (2000) 16: 521-555; Huguenard, J. R., Annu Rev Physiol (1996) 58: 329-348). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall (2000); Huguenard (1996)). T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed α_{1G}, α_{1H}, and α_{1I} (alternately called Cav 3.1, Cav 3.2 and Cav 3.3 respectively).

Calcium channels have been shown to mediate the development and maintenance of the neuronal sensitization and hyperexcitability processes associated with neuropathic pain, and provide attractive targets for the development of analgesic drugs (reviewed in Vanegas, H. & Schaible, H-G., Pain (2000) 85: 9-18). All of the high-threshold calcium channel types are expressed in the spinal cord, and the contributions of L-, N and P/Q-types in acute nociception are currently being investigated. In contrast, examination of the functional roles of these channels in more chronic pain conditions strongly indicates a pathophysiological role for the N-type channel (reviewed in Vanegas & Schaible (2000) supra).

Two examples of either FDA-approved or investigational drugs that act on N-type channels are gabapentin and ziconotide. Ziconotide (Prialt®; SNX-111) is a synthetic analgesic derived from the cone snail peptide Conus magus MVIIA that has been shown to reversibly block N-type calcium channels. In a variety of animal models, the selective block of N-type channels via intrathecal administration of ziconotide significantly depresses the formalin phase 2 response, thermal hyperalgesia, mechanical allodynia and post-surgical pain (Malmberg, A. B. & Yaksh, T. L., J Neurosci (1994) 14: 4882-4890; Bowersox, S. S. et al., J Pharmacol Exp Ther (1996) 279: 1243-1249; Sluka, K. A., J Pharmacol Exp Ther (1998) 287:232-237; Wang, Y-X. et al., Soc Neurosci Abstr (1998) 24: 1626).

Ziconotide has been evaluated in a number of clinical trials via intrathecal administration for the treatment of a variety of conditions including post-herpetic neuralgia, phantom limb syndrome, HIV-related neuropathic pain and intractable cancer pain (reviewed in Mathur, V. S., Seminars in Anesthesia, Perioperative Medicine and Pain (2000) 19: 67-75). In phase II and III clinical trials with patients unresponsive to intrathecal opiates, ziconotide has significantly reduced pain scores and in a number of specific instances resulted in relief after many years of continuous pain. Ziconotide is also being examined for the management of severe post-operative pain as well as for brain damage following stroke and severe head trauma (Heading, C., Curr Opin CPNS Investigational Drugs (1999) 1: 153-166). In two case studies ziconotide has been further examined for usefulness in the management of intractable spasticity following spinal cord injury in patients unresponsive to baclofen and morphine (Ridgeway, B. et al., Pain (2000) 85: 287-289). In one instance, ziconotide decreased the spasticity from the severe range to the mild to none range with few side effects. In another patient, ziconotide also reduced spasticity to the mild range although at the required dosage significant side effects including memory loss, confusion and sedation prevented continuation of the therapy.

Gabapentin, 1-(aminomethyl) cyclohexaneacetic acid (Neurontin™), is an anticonvulsant originally found to be active in a number of animal seizure models (Taylor, C. P. et al., Epilepsy Res (1998) 29: 233-249). Though not specific for N-type calcium channels, subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanical allodynia associated with postoperative pain (Taylor, et al. (1998); Cesena, R. M. & Calcutt, N. A., Neurosci Lett (1999) 262: 101-104; Field, M. J. et al., Pain (1999) 80: 391-398; Cheng, J-K., et al., Anesthesiology (2000) 92: 1126-1131; Nicholson, B., Acta Neurol Scand (2000) 101: 359-371).

While its mechanism of action is not completely understood, current evidence suggests that gabapentin does not directly interact with GABA receptors in many neuronal systems, but rather modulates the activity of high threshold calcium channels. Gabapentin has been shown to bind to the calcium channel α₂δ ancillary subunit, although it remains to be determined whether this interaction accounts for its therapeutic effects in neuropathic pain.

In humans, gabapentin exhibits clinically effective anti-hyperalgesic activity against a wide range of neuropathic pain conditions. Numerous open label case studies and three large double blind trials suggest gabapentin might be useful in the treatment of pain. Doses ranging from 300-2400 mg/day were studied in treating diabetic neuropathy (Backonja, M. et al., JAMA (1998) 280:1831-1836), postherpetic neuralgia (Rowbotham, M. et al., JAMA (1998) 280: 1837-1842), trigeminal neuralgia, migraine and pain associated with cancer and multiple sclerosis (Di Trapini, G. et al., Clin Ter (2000) 151: 145-148; Caraceni, A. et al., J Pain & Symp Manag (1999) 17: 441-445; Houtchens, M. K. et al., Multiple Sclerosis (1997) 3: 250-253; see also Magnus, L., Epilepsia (1999) 40(Suppl 6): S66-S72; Laird, M. A. & Gidal, B. E., Annal Pharmacotherap (2000) 34: 802-807; Nicholson, B., Acta Neurol Scand (2000) 101: 359-371).

T-type calcium channels are involved in various medical conditions. In mice lacking the gene expressing the α_{1G} subunit, resistance to absence seizures was observed (Kim, C. et al., Mol Cell Neurosci (2001) 18(2): 235-245). Other studies have also implicated the α_{1H} subunit in the development of epilepsy (Su, H. et al., J Neurosci (2002) 22: 3645-3655). There is strong evidence that some existing anticonvulsant drugs, such as ethosuximide, function through the blockade of T-type channels (Gomora, J. C. et al., Mol Pharmacol (2001) 60: 1121-1132).

Low voltage-activated calcium channels are highly expressed in tissues of the cardiovascular system. Mibefradil, a calcium channel blocker 10-30 fold selective for T-type over L-type channels, was approved for use in hypertension and angina. It was withdrawn from the market shortly after launch due to interactions with other drugs (Heady, T. N., et al., Jpn J. Pharmacol. (2001) 85:339-350).

Growing evidence suggests T-type calcium channels are also involved in pain (see for example: US Patent Application No. 2003/086980; PCT Patent Application Nos. WO 03/007953 and WO 04/000311). Both mibefradil and ethosuximide have shown anti-hyperalgesic activity in the spinal nerve ligation model of neuropathic pain in rats (Dogrul, A., et al., Pain (2003) 105:159-168). In addition to cardiovascular disease, epilepsy (see also US Patent Application No. 2006/025397), and chronic and acute pain, T-type calcium channels have been implicated in diabetes (US Patent Application No. 2003/125269), certain types of cancer such as prostate cancer (PCT Patent Application Nos. WO 05/086971 and WO 05/77082), sleep disorders (US Patent Application No. 2006/003985), Parkinson's disease (US Patent Application No. 2003/087799); psychosis such as schizophrenia (US Patent Application No. 2003/087799), overactive bladder (Sui, G.-P., et al., British Journal of Urology International (2007) 99(2): 436-441; see also US 2004/197825) and male birth control.

The present invention provides novel compounds having calcium channel activity, and which are active as inhibitors of N-type calcium channels in particular. These compounds are thus useful for treatment of disorders including pain and certain mood disorders, gastrointestinal disorders, genitourinary disorders, neurologic disorders and metabolic disorders.

### SUMMARY OF THE INVENTION

The invention relates to compounds useful in treating conditions modulated by calcium channel activity and in particular conditions mediated by N-Type and/or T-type channel activity. The compounds of the invention are substituted or unsubstituted cyclylamine derivatives with structural features that enhance the calcium channel blocking activity of the compounds.

Thus, in one aspect, the invention is directed to a method of treating conditions mediated by calcium channel activity by administering to patients in need of such treatment at least one compound of formula (1):

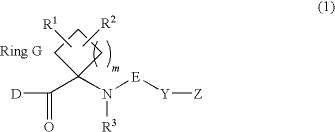

or a pharmaceutically acceptable salt or conjugate thereof, wherein

m is 0-3;
Ring G optionally contains O, S or NR as a ring member in place of one carbon atom, wherein
   R is independently H or optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

wherein one or more optional substituents on R are selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,

wherein each R′ is H or independently C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S as ring members;

R¹ and R² are independently selected from a group consisting of H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl group, halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, and NO₂, wherein R is defined above; and two R can be linked to form a 3-8 membered ring, optionally containing one or two N, O or S as a ring member; wherein one or more optional substituents on each R and each ring formed by linking two R groups together, are selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein R′ is defined above;

or R¹ and R² are optionally connected together to form an optionally substituted 5-6 membered ring fused to ring G; optionally containing one or more N, O or S; and wherein the ring formed by linking R¹ and R² groups together, is optionally substituted with one or more substituents selected from optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, halo, or is selected from OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, NO₂, ═CR′₂, ═O, ═N—CN, ═N—OR′, or ═NR′, wherein R and R′ is defined as described above;

R³ is H or C1-C4 alkyl, C2-C4 alkenyl, or C2-C4 alkynyl, each of which is substituted with one or more ═O, halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, and NO₂, wherein R is defined as described above;

E is —C(═O)— or C1-C4 alkylene, optionally substituted with one or more C1-C8 alkyl, C2-C8 alkenyl, C2-C8 alkynyl, C1-C8 acyl, C6-C10 aryl, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, NO₂, halo, or ═O, wherein each R is defined above;

D is OH or D is NR⁴R⁵;

R⁴ is H and R⁵ is optionally substituted C1-C4 alkyl or -L-Q, wherein

L is a bond or C1-C4 alkylene optionally substituted with one or more C1-C8 alkyl, C2-C8 alkenyl, C2-C8 alkynyl, C1-C8 acyl, C6-C10 aryl, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, NO₂, halo, and ═O, wherein each R is defined above;

Q is an optionally substituted 5-6 membered ring that may contain up to 4 heteroatoms as ring members, each independently selected from O, S, N and NR⁶, wherein R⁶ is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or SO₂R⁷, each of which is optionally substituted with up to four groups selected from R⁷, halo, CN, OR⁷, ═O, C(NR⁷)NR⁷₂, NR⁷₂, COR⁷, COOR⁷, CONR⁷₂, SR⁷, SOR⁷, SO₂R⁷, SO₂NR⁷₂, NR⁷COOR⁷, and COCOOR⁷, wherein

each R⁷ is independently H or C1-C8 alkyl, C2-C8 heteroalkyl, C7-C12 arylalkyl, or diarylalkyl, each of which may be optionally substituted, and wherein two R⁷ groups can cyclize to form a 3 to 8 membered ring, optionally including up to two heteroatoms selected from N, O and S as ring members;

or R⁴ and R⁵ are joined to form an optionally substituted 5-6 membered saturated ring, which may contain up to 2 heteroatoms selected from NR⁶, O and S as ring members, wherein each R⁶ is described as above;

Y is a bond or C1-C3 alkylene optionally substituted with ═O;

Z is an optionally substituted 5-6 membered aromatic ring; and with the proviso wherein
if D is 4-substituted aniline, R³ is H, and Y is a bond, then Z is not thiophenyl;
Z is not a substituted 9-membered bicyclic group comprised of a fused pyrazolyl and pyrimidinyl moiety;
if D is OH or D is NR⁴R⁵, wherein R⁴ is not H, then R³ is not an unsubstituted C1-C4 alkyl;
if Z is 2-(quinolin-5-yl)oxazole and E is C═O, then Y is not a bond; and
if E is ═O and Y is a bond, then Z is not a substituted 9-membered bicyclic ring comprising an imidazole.

One aspect of the invention relates to a novel compound selected from the group in FIG. 1.

Another aspect of the invention includes a pharmaceutical composition which comprises the compound of formula 1 in admixture with a pharmaceutically acceptable excipient. The compounds of the invention may also be in the form of a salt if appropriate, or in the form of a prodrug.

One aspect of the invention includes a method to treat a condition mediated by N-type or T-type calcium ion channels. The method comprises administering to a subject in need of such treatment an amount of the compound of formula 1 or dual active compounds that selectively affect N-type and/or T-type channels or a pharmaceutical composition thereof effective to ameliorate said condition. An example of said condition is chronic or acute pain, mood disorders, neurodegenerative disorders, gastrointestinal disorders, genitourinary disorders, neuroprotection, metabolic disorders, cardiovascular disease, epilepsy, diabetes, prostate cancer, sleep disorders, Parkinson's disease, schizophrenia or male birth control. A preferred example of said condition is chronic or acute pain.

In particular examples, compounds having formula 1 contain at least one chiral center. The compounds may be in the form of isolated stereoisomers or mixtures of various stereoisomers, including enantiomeric mixtures, equimolar mixtures of all possible stereoisomers, or various degrees of chiral or optical purity.

The invention also relates to methods of antagonizing calcium channel activity using the compounds of formula 1, thus treating conditions associated with calcium channel activity. For example, compounds for formula 1 may be used for treating conditions associated with undesired calcium channel activity. Alternatively, compounds of formula 1 may be used to treat a subject that may have normal calcium channel function which nevertheless results in an undesirable physical or metabolic state.

In one aspect, the invention relates to methods for modulating calcium channel activity in a subject, comprising administering a compound of formula 1, or a pharmaceutical composition thereof, to a subject in need of such treatment. In another aspect, the invention relates to methods for ameliorating pain in a subject, comprising administering a compound of claim 1 or a pharmaceutical composition thereof to a subject in need of such treatment.

Furthermore, the invention relates to combinatorial libraries containing the compounds of formula 1. The invention also relates to methods for screening such libraries for members containing particularly potent calcium channel blocking activity, or for members that antagonize one type of such channels specifically.

The invention is also directed to the use of compounds of formula (1) for the preparation of medicaments for the treatment of conditions requiring modulation of calcium channel activity, and in particular N-type and/or T-type calcium channel activity. In another aspect, the invention is directed to pharmaceutical compositions containing compounds of formula (1) and to the use of these compositions for treating conditions requiring modulation of calcium channel activity, and particularly N-type and/or T-type calcium channel activity. The invention is also directed to compounds of formula (1) useful to modulate calcium channel activity, particularly N-type and/or T-type channel activity.

The invention also provides methods for using such compounds in treating conditions such as stroke, anxiety, overactive bladder, inflammatory bowel disease, head trauma, migraine, chronic, neuropathic and acute pain, epilepsy, hypertension, cardiac arrhythmias, and other indications associated with calcium metabolism, including synaptic calcium channel-mediated functions. For example, selective N-type calcium channel blockers are particularly useful for treating pain, stroke, anxiety, epilepsy, inflammatory bowel disease and overactive bladder. Selective N-type and/or T-type calcium channel blockers are useful for treating epilepsy, cardiovascular disease and pain. Dual blockers of both N-type and T-type channels would be especially useful for treating epilepsy, stroke and some forms of pain.

### BRIEF DESCRIPTION OF THE DRAWINGS

FIG. 1 shows the structures of illustrative compounds of the invention.

### DETAILED DESCRIPTION

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-8C (alkyl) or 2-8C (alkenyl or alkynyl). In some embodiments, they contain 1-6C, 1-4C, 1-3C or 1-2C (alkyl); or 2-6C, 2-4C or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a IC alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N.

The designated number of carbons in heteroforms of alkyl, alkenyl and alkynyl includes the heteroatom count. For example, if heteroalkyl is defined as 1-6C, it will contain 1-6 C, N, O, or S atoms such that the heteroalkyl contains at least one C atom and at least one heteroatom, for example 1-5C and 1N or 1-4C and 2N. Similarly, when heteroalkyl is defined as 1-6C or 1-4C, it would contain 1-5C or 1-3C respectively, i.e., at least one C is replaced by O, N or S. Accordingly, when heteroalkenyl or heteroalkynyl is defined as 2-6C (or 2-4C), it would contain 2-6 or 2-4 C, N, O, or S atoms, since the heteroalkenyl or heteroalkynyl contains at least one carbon atom and at least one heteroatom, e.g. 2-5C and 1N or 2-4C and 20. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙOR, (CH₂)ₙCOR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the group contains at least one C and the size of the substituent is consistent with the definition of alkyl, alkenyl and alkynyl.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, 2-, 3- or 4-pyridyl, indolyl, 2- or 4-pyrimidyl, pyridazinyl, benzothiazolyl or benzimidazolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C, 1-6C or more particularly 1-4C or 1-3C when saturated or 2-8C, 2-6C, 2-4C or 2-3C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, or NR′SO₂R′, wherein each R′ is independently H or an optionally substituted group selected from alkyl, alkenyl, alkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group, are typically selected from the same list of substituents suitable for aromatic or heteroaromatic groups and may further be selected from ═O and ═NOR′ where R′ is H or an optionally substituted group selected from alkyl, alkenyl, alkynyl, heteroaryl, and aryl (all as defined above).

Halo may be any halogen atom, especially F, Cl, Br, or I, and in preferred embodiments it is fluoro, or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

A preferred embodiment of R³ is H.

In some preferred embodiments, E is —C(═O)—, —CH₂—, —CH₂CH₂— or —CH₂CH₂CH₂—.

In some preferred embodiments, Y is a bond, —CH₂—, —CH₂CH₂— or —CH₂CH₂CH₂—.

In some preferred embodiments, Z is optionally substituted phenyl or pyridinyl ring. Preferred optional substitutents on Z include CF₃, methyl, ethyl, propyl, t-butyl, OH, cyclopropyl, OMe, C1-C3 cyanoalkyl, and CMe₂CONH₂.

In some embodiments, D is OH. In other embodiments, D is NR⁴R⁵. In some preferred embodiments R⁴ is H and R⁵ is methyl, ethyl, propyl or t-butyl.

In other embodiments, R⁵ is L-Q. In some preferred embodiments, L is a bond, —CH₂—, —CH₂CH₂— or —CH₂CH₂CH₂—.

Preferred embodiments of Q include phenyl, pyrimidinyl, pyridinyl, pyrazinyl, triazinyl, furanyl, oxadiazolyl, oxazolyl, isoxazolyl, pyrazolyl, thiazolyl, thiophenyl, thiadiazolyl, isothiazolyl, indazolyl, indolyl, morpholinyl and benzimidazolyl. Particularly preferred embodiments of Q include phenyl, pyridinyl, pyrazinyl, isoxazolyl, pyrazolyl, thiazolyl, morpholinyl and benzimidazolyl.

Preferred optional substituents of Q include up to four substituents independently selected from the group consisting of CF₃, C1-C6 alkyl, C1-C6 alkoxy, heterocyclylalkyl, —SO₂R⁸, halo, and -L′NR⁹R⁹, wherein L′ is a bond or optionally substituted C1-C4 alkylene, R⁸ is H or C1-C4 alkyl; and each R⁹ is independently selected from a group consisting of H or C1-C8 alkyl, C1-C8 alkenyl, C2-C8 heteroalkyl, C2-C8 heteroalkenyl, C3-C8 cyclylalkyl, C3-C8 heterocyclylalkyl, C6-C10 aryl, C7-C12 arylalkyl, C4-C12 heteroaryl, C6-C12 heteroarylalkyl, and —SO₂R⁸, each of which is optionally substituted, or two R⁹ on the same nitrogen may form an optionally substituted 5-6 membered ring optionally containing O or NR⁸ as a ring member.

Particularly preferred embodiments of L′ include —CH₂—, —CH₂CH₂— or —CH₂CH₂CH₂-Even more particularly preferred substituents of Q include up to three substituents independently selected from the group CF₃, methyl, ethyl butyl, t-butyl, C1-C3 morpholinoalkyl, N-methylpiperazinylalkyl, CMe₂CN, Cl, F, benzyl, phenyl, SO₂Me, OMe, NMe₂, and CMe₂CONH₂.

In preferred embodiments, R⁴ and R⁵ are joined to form a 5-6 membered saturated ring. In particularly preferred embodiments, R⁴ and R⁵ are joined to form optionally substituted piperidinyl, piperazinyl, pyrrolidinyl or morpholinyl. Preferred optional substituents on the formed ring include one or more optionally substituted methyl, ethyl, propyl, t-butyl, benzyl, or phenyl.

A preferred embodiment of m is 2

In some preferred embodiments, R¹ and R² are optionally connected together to form an optionally substituted 6-membered aromatic group with said ring G. In particularly preferred embodiments, R¹ and R² are attached to adjacent atoms of said ring G, and are joined to form a phenyl ring fused to said ring G. Preferred optional substituents of the phenyl ring include one or more C1-C6 alkyl, halo, CF₃, OCF₃, NO₂, NR¹⁰₂, OR¹⁰, SR¹⁰, COR¹⁰, COOR¹⁰, CONR¹⁰₂, NR¹⁰OCR¹⁰ or OOCR¹⁰, wherein R¹⁰ is H or C1-C4 alkyl, or two R¹⁰ attached to the same N may be joined to form an optionally substituted 5-7 membered ring.

In some preferred embodiments, ring G contains a heteroatom O or NR as a ring member, wherein R is —COOR¹¹ or R¹¹, wherein R¹¹ is H or C1-C8 alkyl. Particularly preferred embodiments of NR include NCO₂tBu, NH, and NMe, NCH₂CH₃, NCH₂CH₂CH₃, Nt-butyl.

In preferred embodiments, Z is phenyl, pyridinyl, pyrazinyl, or pyrimidinyl. In particularly preferred embodiments, Z is phenyl or pyridinyl. Preferred optional substituents of Z include up to four substituents independently selected from the group consisting of —CF₃, —OH, —CN, halo, C3-C8 cycloalkyl, C1-C6 alkoxy and C1-C6 alkyl, wherein C3-C8 cycloalkyl, C1-C6 alkoxy, and C1-C6 alkyl are optionally substituted with halo, —OR, —CN, —COOR¹² or —CONR¹²₂, wherein each R¹² is independently selected from H and C1-C6 alkyl. Particularly preferred optional substituents of Z include up to four substituents independently selected from the group consisting of —CF₃, —OH, —CN, t-Bu, cyclopropyl, C2-C4 cyanoalkyl, OR¹¹ and C1-C4 alkyl, wherein C1-C4 alkyl is optionally substituted with —CONR¹¹₂, wherein each R¹¹ is independently selected from H and C1-C6 alkyl.

The compounds of the invention may be in the form of pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Compounds of formula (1) are also useful for the manufacture of a medicament useful to treat conditions characterized by undesired N-type and/or T-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

Compounds for this invention are often selelective for N or T-type calcium channels. Preferred compounds are especially selective relative to L-type and P/Q-type channels. Selective ones preferably have an IC₅₀ of at least 10× lower on one type of calcium channel.

### MODES OF CARRYING OUT THE INVENTION

The compounds of formula (1) are useful in the methods of the invention and exert their desirable effects through their ability to modulate the activity of calcium channels, particularly the activity of N-type and/or T-type calcium channels. This makes them useful for treatment of certain conditions where modulation of N-type calcium channels is desired, including: chronic and acute pain; mood disorders such as anxiety, depression, and addiction; neurodegenerative disorders; hearing disorders; gastrointestinal disorders such as inflammatory bowel disease and irritable bowel syndrome; genitourinary disorders such as urinary incontinence, interstitial colitis and sexual dysfunction; neuroprotection such as cerebral ischemia, stroke and traumatic brain injury; and metabolic disorders such as diabetes and obesity. Certain conditions where modulation of T-type calcium channels is desired includes: cardiovascular disease; epilepsy; diabetes; certain types of cancer such as prostate cancer; pain, including both chronic and acute pain; sleep disorders; Parkinson's disease; psychosis such as schizophrenia; overactive bladder and male birth control.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

Anxiety as used herein includes but is not limited to the following conditions: generalized anxiety disorder, social anxiety disorder, panic disorder, obsessive-compulsive disorder, and post-traumatic stress syndrome. Addiction includes but is not limited to dependence, withdrawal and/or relapse of cocaine, opioid, alcohol and nicotine.

Neurodegenerative disorders as used herein include Parkinson's disease, Alzheimer's disease, multiple sclerosis, neuropathies, Huntington's disease, presbycusis and amyotrophic lateral sclerosis (ALS).

Cardiovascular disease as used herein includes but is not limited to hypertension, pulmonary hypertension, arrhythmia (such as atrial fibrillation and ventricular fibrillation), congestive heart failure, and angina pectoris.

Epilepsy as used herein includes but is not limited to partial seizures such as temporal lobe epilepsy, absence seizures, generalized seizures, and tonic/clonic seizures.

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

It is known that calcium channel activity is involved in a multiplicity of disorders, and particular types of channels are associated with particular conditions. The association of N-type and/or T-type channels in conditions associated with neural transmission would indicate that compounds of the invention which target N-type and/or T-type receptors are most useful in these conditions. Many of the members of the genus of compounds of formula (1) exhibit high affinity for N-type and/or T-type channels. Thus, as described below, they are screened for their ability to interact with N-type and/or T-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

There are three distinguishable types of calcium channel inhibition. The first, designated “open channel blockage,” is conveniently demonstrated when displayed calcium channels are maintained at an artificially negative resting potential of about −100 mV (as distinguished from the typical endogenous resting maintained potential of about −70 mV). When the displayed channels are abruptly depolarized under these conditions, calcium ions are caused to flow through the channel and exhibit a peak current flow which then decays. Open channel blocking inhibitors diminish the current exhibited at the peak flow and can also accelerate the rate of current decay.

This type of inhibition is distinguished from a second type of block, referred to herein as “inactivation inhibition.” When maintained at less negative resting potentials, such as the physiologically important potential of −70 mV, a certain percentage of the channels may undergo conformational change, rendering them incapable of being activated—i.e., opened—by the abrupt depolarization. Thus, the peak current due to calcium ion flow will be diminished not because the open channel is blocked, but because some of the channels are unavailable for opening (inactivated). “Inactivation” type inhibitors increase the percentage of receptors that are in an inactivated state.

A third type of inhibition is designated “resting channel block”. Resting channel block is the inhibition of the channel that occurs in the absence of membrane depolarization, that would normally lead to opening or inactivation. For example, resting channel blockers would diminish the peak current amplitude during the very first depolarization after drug application without additional inhibition during the depolarization.

In order to be maximally useful in treatment, it is also helpful to assess the side reactions which might occur. Thus, in addition to being able to modulate a particular calcium channel, it is desirable that the compound has very low activity with respect to the hERG K⁺ channel which is expressed in the heart. Compounds that block this channel with high potency may cause reactions which are fatal. Thus, for a compound that modulates the calcium channel, it should also be shown that the hERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Assay Examples 1-4.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, e.g., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to activate or block the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to block the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Accordingly, a library of compounds of formula (1) or formula (2) can be used to identify a compound having a desired combination of activities that includes activity against at least one type of calcium channel. For example, the library can be used to identify a compound having a suitable level of activity on N-type calcium channels while having minimal activity on HERG K+ channels.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) or (2) may be used alone, as mixtures of two or more compounds of formula (1) and/or (2) or in combination with other pharmaceuticals. An example of other potential pharmaceuticals to combine with the compounds of formula (1) would include pharmaceuticals for the treatment of the same indication but having a different mechanism of action from N-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

The compounds of the invention may be prepared and used as pharmaceutical compositions comprising an effective amount of at least one compound of formula (1) admixed with a pharmaceutically acceptable carrier or excipient, as is well known in the art. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.01-15 mg/kg, preferably 0.1-10 mg/kg. However, dosage levels are highly dependent on the nature of the condition, drug efficacy, the condition of the patient, the judgment of the practitioner, and the frequency and mode of administration. Optimization of the dosage for a particular subject is within the ordinary level of skill in the art.

Synthesis of the Invention Compounds

The following reaction schemes and examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

A variety of synthetic methods familiar to those skilled in the art of Organic Chemistry may be employed in the preparation of compounds of Formula 1. In this discussion it will be recognized by a skilled practitioner that a sequence proposed for one series of compounds may require minor modifications, such as a re-ordering of synthetic steps, the use of different reaction conditions or reagents, or the selection of an alternative protecting group scheme, to be effective in producing the desired analog of Formula 1. References describing the use and limitations of protecting groups can be found in Greene and Wuts, Protective Groups in Organic Synthesis, Wiley-Interscience. References describing synthetic transformations can be found in Larock, Comprehensive Organic Transformations, Wiley-VCH. It is understood, however, that these compendia contain only some of the protecting groups and synthetic reactions that are available to one skilled in the art to prepare the compounds of Formula 1.

### Example 1

### Synthesis of Intermediates

### 1(a) Synthesis of 3,5-dicyclopropylbenzoic acid

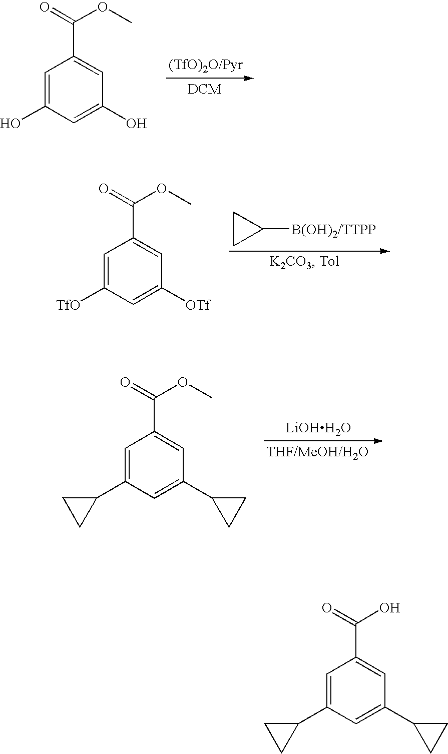

### Preparation of methyl 3,5-bis(trifluoromethylsulfonyloxy)benzoate

Methyl-3,5-dihydroxybenzoate (2 g, 11.9 mmol) and pyridine (1.9 g, 23.8 mmol) were stirred in DCM at 0° C. Triflic anhydride (5.2 g, 19 mmol) was added and the mixture allowed to warm to rt. After 2 h, the reaction was diluted with Et₂O (50 mL), quenched with 10% HCl, washed with saturated NaHCO₃ and the organics concentrated in-vacuo to give methyl 3,5-bis(trifluoromethylsulfonyloxy)benzoate (amt, 66%). (MS m/z 432, calc'd for C₁₀H₆F₆O₈S₂ 432.3). The product was used without further purification.

### Preparation of methyl 3,5-dicyclopropylbenzoate

Methyl 3,5-bis(trifluoromethylsulfonyloxy)benzoate (300 mg, 0.69 mmol), K₂CO₃ (400 mg, 2.9 mmol), cyclopropyl boric acid (356 mg, 4.1 mmol) and tetrakistriphenylphosphine (160 mg, 0.13 mmol) were refluxed in toluene (20 mL) for 16 h. The reaction was cooled, filtered, concentrated in-vacuo and the residue purified by column chromatography (hexane/EtOAc 15/1) to give methyl 3,5-dicyclopropylbenzoate (90 mg, 60%). (MS m/z 216, calc'd for C₁₄H₁₆O₂ 216.3).

### Preparation of 3,5-dicyclopropylbenzoic acid

Methyl 3,5-dicyclopropylbenzoate, (100 mg, 0.46 mmol) and LiOH.H₂O (40 mg, 0.97 mmol) were stirred in THF/MeOH/H₂O (5 mL, 3/1/1) for 16 h. The reaction was concentrated in-vacuo, the residue dissolved in H₂O (2 mL), acidified with 10% HCl and the resultant solid collected by filtration to give 3,5-dicyclopropylbenzoic acid (86 mg, 92%). The product was used without further purification (MS m/z 202, calc'd for C₁₃H₁₄O₂ 202.3).

### 1(b) Synthesis 3,5-di-tert-butyl-4-methoxy benzoic acid

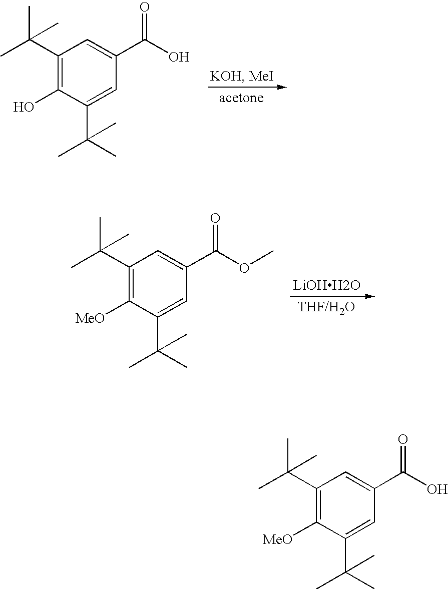

S 3,5-di-tert-butyl-4-hydroxy benzoic acid (50 g, 199 mmol), KOH (28 g, 499 mmol) and MeI (37 mL, 599 mmol) were stirred in acetone (1 L) at rt for 18 h. The reaction mixture was concentrated in-vacuo and the residue partitioned between EtOAc and H₂O. The aqueous phase was extracted three times with EtOAc and the organics combined, dried (Na₂SO₄) and concentrated in-vacuo. The crude residue was stirred in THF/H₂O (1/1) (500 mL) with LiOH.H₂O (25 g, 595 mmol) for 18 h at rt. The reaction was concentrated in-vacuo and the resultant solution acidified with conc. HCl. The product was recovered by filtration to give 3,5-di-tert-butyl-4-methoxybenzoic acid (27 g, 51% from 5). (1H NMR (400 mHz, CDCl₃) δ 1.47 (s, 18H), 3.74 (s, 3H), 8.04 (s, 2H). MS m/z 263.1 (calcd for C16H24O3, 264.4)

### 1(c) Synthesis of 4-(2-cyanopropan-2-yl)benzoic acid

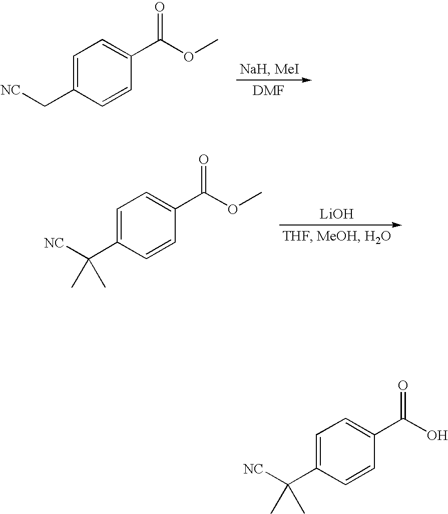

### Preparation of methyl 4-(2-cyanopropan-2-yl)benzoate

Methyl 4-(cyanomethyl)benzoate (5 g, 28.5 mmol) was stirred in DMF (50 mL) at 0° C. and NaH (3.44 g, 85.7 mmol) added in portions. MeI (5.35 mL, 85.7 mmol) in DMF (20 mL) was added dropwise over 30 min and the reaction stirred at rt for 18 h. The reaction was quenched with H₂O (20 mL), extracted with EtOAc (3×20 mL) and the combined organics washed sequentially with 1 M HCl (2×20 mL) and saturated NaHCO₃ solution (20 mL) and dried over MgSO₄. The crude mixture was concentrated in-vacuo and the residue purified by column chromatography (20% EtOAc/Petroleum ether), to give methyl 4-(2-cyanopropan-2-yl)benzoate (4.22 g, 73%).

### Preparation of 4-(2-cyanopropan-2-yl)benzoic acid

Methyl 4-(2-cyanopropan-2-yl)benzoate (0.2 g, 0.72 mmol) and LiOH.H₂O (0.045 g, 1.08 mmol) were stirred in THF/H₂O/MeOH (3/1/1) (5 mL) at rt for 18 h. The organic solvents were removed in-vacuo and the aqueous solution acidified (to pH 2) with conc. HCl. The product was recovered by filtration to give 4-(2-cyanopropan-2-yl)benzoic acid (0.18 g, 89%). The product was used without further purification (MS m/z [M−H] 188.3 calc'd for C₁₁H₁₁NO₂ 189.2).

### 1(d) Synthesis of 3,5-bis(2-cyanopropan-2-yl)benzoic acid

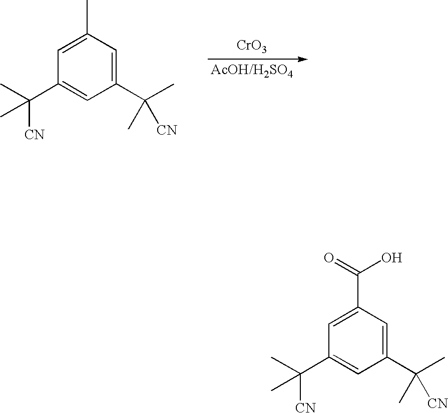

2,2′-(5-Methyl-1,3-phenylene)bis(2-methylpropanenitrile) (2.26 g, 10 mmol) was dissolved in AcOH (20 mL) and conc. H₂SO₄ (1.5 mL) at 0° C. CrO₃ (3 g, 30 mmol) was added in portions, the mixture stirred at 0° C. for 2 h then diluted with H₂O (60 mL). The aqueous solution was extracted with EtOAc (40 mL) and the organics washed with brine, dried (Na₂SO₄) and concentrated in-vacuo. The residue was purified by column chromatography (2% MeOH/DCM) to give 3,5-bis(2-cyanopropan-2-yl)benzoic acid (2.0 g, 78%). MS m/z 255.4 (calcd C₁₅H₁₆N₂O₂, 256.12).

### 1(e) Synthesis of 3-t-butyl-5-(2-cyanopropan-2-yl)benzoic acid

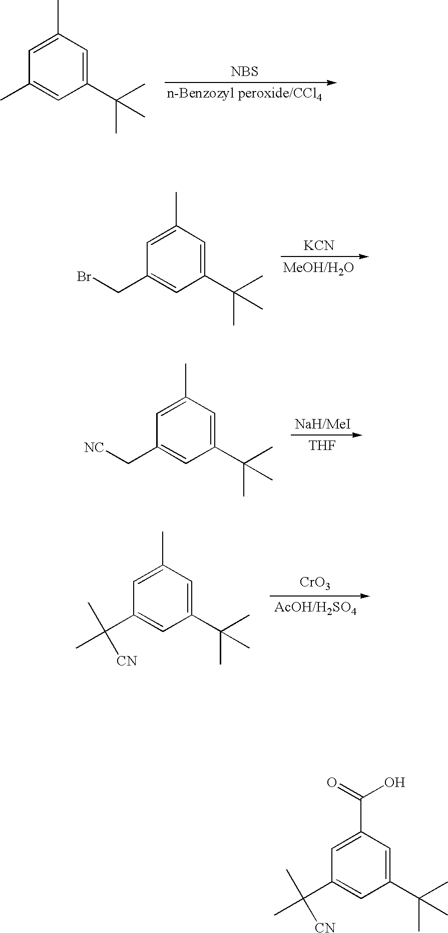

### Preparation of 1-(bromomethyl)-3-tert-butyl-5-methylbenzene

5-t-butyl-m-xylene (5 g, 30.81 mmol), NBS (4.39 g, 24.65 mmol) and benzoyl peroxide (0.1 g, 0.3 mmol) were refluxed in CCl₄ (100 mL) for 16 h. The reaction was cooled, filtered and concentrated in-vacuo and the residue purified by column chromatography (100% pet ether) to give 1-(bromomethyl)-3-tert-butyl-5-methylbenzene (5.4 g, 73%).

### Preparation of 2-(3-tert-butyl-5-methylphenyl)acetonitrile

1-(bromomethyl)-3-tert-butyl-5-methylbenzene (5.4 g, 22.5 mmol) and KCN (2.19 g, 33.75 mmol) were refluxed in MeOH:H₂O (9:1, 100 mL) for 20 h. The reaction was cooled, concentrated in-vacuo and the residue was taken up in EtOAc (50 mL). The organics were washed with brine (1×40 mL), dried (Na₂SO₄), concentrated in-vacuo and the residue purified by column chromatography (pet ether:EtOAc, 25:1) to give 2-(3-tert-butyl-5-methylphenyl)acetonitrile, (1.55 g, 26.90%).

### Preparation of 2-(3-tert-butyl-5-methylphenyl)-2-methylpropanenitrile

2-(3-tert-butyl-5-methylphenyl)acetonitrile (1.55 g, 8.29 mmol) was dissolved in anhydrous THF (40 mL) under N₂. NaH (60% dispersion in mineral oil, 1 g, 24.9 mmol) was added in portions. MeI (1.55 mL, 24.9 mmol) was added and the reaction stirred at rt for 16 h. The reaction was quenched with saturated NH₄Cl solution (10 mL), extracted with EtOAc (2×40 mL) and the combined organic extracts washed with brine (50 mL), dried (Na₂SO₄) and concentrated in-vacuo. The residue was purified by column chromatography (pet ether:EtOAc, 25:1) to give 2-(3-tert-butyl-5-methylphenyl)-2-methylpropanenitrile (16) (1.37 g, 76.9%).

### Preparation of 3-tert-butyl-5-(2-cyanopropan-2-yl)benzoic acid

2-(3-tert-butyl-5-methylphenyl)-2-methylpropanenitrile (1.37 g, 6.37 mmol) was dissolved in AcOH (13 mL) and conc H₂SO₄ (1 mL) at 0° C. CrO₃ (1.911 g, 1.9 mmol) was added in portions and stirred at 0° C. for 2 h. The reaction was diluted with H₂O (40 mL), extracted with EtOAc (40 mL), washed with brine, dried (Na₂SO₄) and concentrated in-vacuo. The residue was purified by column chromatography (2% MeOH:DCM) to give 3-tert-butyl-5-(2-cyanopropan-2-yl)benzoic acid (0.75 g, 48%). MS m/z 244.5 (calcd C₁₅H₁₉NO₂, 245.32).

### 1(f) Synthesis of 3-(2-cyanopropan-2-yl)-4-methoxybenzoic acid

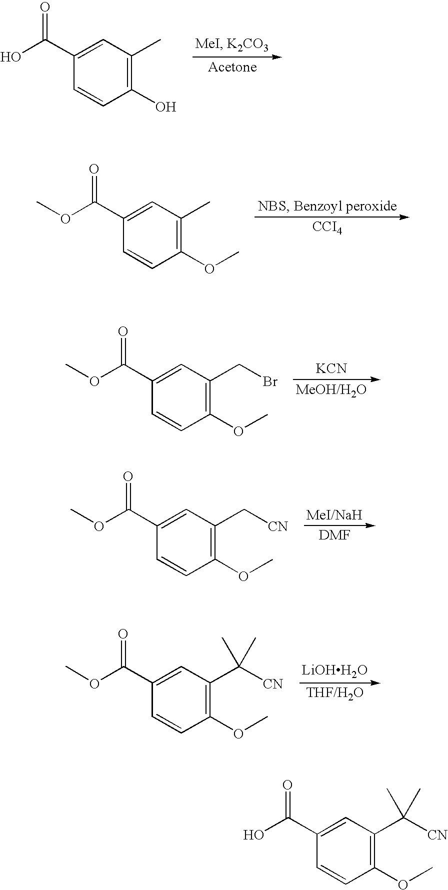

### Preparation of methyl 4-methoxy-3-methylbenzoate

4-Hydroxy-3-methylbenzoic acid (10 g, 65.7 mmol), K₂CO₃ (22.5 g, 163.2 mmol) and MeI (10.1 mL, 163.2 mmol) were heated at reflux for 16 h. The reaction was cooled and partioned between EtOAc and H₂O. The aqueous layer was extracted with EtOAc and the combined organics concentrated in-vacuo to give methyl 4-methoxy-3-methylbenzoate (11.7 g, 65.2 mmol). (1H NMR 300 mHz, CDCl₃) δ 2.16 (s, 3H), 3.80 (s, 6H), 6.77 (d, 1H, J=8.7 Hz), 7.75 (s, 1H), 7.83 (d, 1H, J=8.7 Hz). MS m/z 181.4 (calcd for C10H₁₂O₃). The product was without further purification.

### Preparation of methyl 3-(bromomethyl)-4-methoxybenzoate

Methyl 4-methoxy-3-methylbenzoate, NBS (14.8 g, 83.3 mmol) and benzoyl peroxide (5.1 g, 21.0 mmol) were refluxed in CCl₄ for 16 h. The reaction was cooled, filtered, concentrated in-vacuo and purified by Biotage (10% EtOAc/Pet ether) to give methyl 3-(bromomethyl)-4-methoxybenzoate (12.06 g, 71%). (1H NMR 300 mHz, CDCl₃) δ 3.90 (s, 3H), 3.97 (s, 3H), 4.56 (s, 2H), 6.92 (d, 1H, J=8.4 Hz), 7.10 (s, 1H), 8.02 (d, 1H, J=8.4 Hz).

### Preparation of methyl 3-(cyanomethyl)-4-methoxybenzoate

Methyl 3-(bromomethyl)-4-methoxybenzoate (2.76 g, 10.7 mmol) and KCN (1.04 g, 16.05 mmol) were refluxed in MeOH:H₂O (9:1, 40 mL) for 16 h. The reaction was concentrated in-vacuo, the residue taken up in Et₂O (50 mL), washed with H₂O, dried (MgSO₄) concentrated in-vacuo and the product purified by Biotage (20% EtOAc/pet ether) to give methyl 3-(cyanomethyl)-4-methoxybenzoate, (1.08 g, 49.8%). (1H NMR 300 mHz, CDCl₃) δ 3.70 (s, 2H), 3.91 (s, 3H), 3.95 (s, 3H), 6.94 (d, 1H, J=9 Hz), 8.06 (m, 2H).

### Preparation of methyl 3-(2-cyanopropan-2-yl)-4-methoxybenzoate

Methyl 3-(cyanomethyl)-4-methoxybenzoate (1.08 g, 5.3 mmol) was stirred in DMF (10 mL) at 0° C. under N₂ and NaH (60% dispersion in mineral oil, 588 mg, 14.7 mmol) was added in portions. MeI (0.92 mL, 14.7 mmol) in DMF (10 mL) was added dropwise and the reaction allowed to warm to rt and stirred for 3 h. The reaction was partitioned between EtOAc and H₂O. The organics were washed sequentially with H₂O and brine, dried (MgSO₄), concentrated in-vacuo and purified by Biotage (20% EtOAc pet ether) to give methyl 3-(2-cyanopropan-2-yl)-4-methoxybenzoate (1.01 g, 81.3%). %). (1H NMR 300 mHz, CDCl₃) δ 1.79 (s, 6H), 3.92 (s, 3H), 4.01 (s, 3H), 6.98 (d, 1H, J=8.7 Hz), 8.06 (m, 2H).

### Preparation of 3-(2-cyanopropan-2-yl)-4-methoxybenzoic acid

Methyl 3-(2-cyanopropan-2-yl)-4-methoxybenzoate (500 mg, 2.14 mmol) and LiOH.H₂O (134 mg, 3.21 mmol) were stirred in THF/H₂O (10 mL, 1:1) at rt for 16 h. The organic solvent was removed in-vacuo, the aqueous layer made pH 3 (1M HCl) and extracted with EtOAc. The organics were dried (MgSO₄) and concentrated in-vacuo to give 3-(2-cyanopropan-2-yl)-4-methoxybenzoic acid, (460 mg, 98%). MS (neg ion mode) m/z 218.4 (calc'd for C₁₂H₁₃NO₃). The product was used without further purification

### 1(g) Synthesis of 2-(2-cyanopropan-2-yl)isonicotinic acid

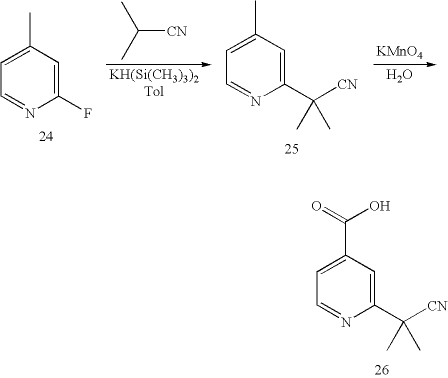

### Preparation of 2-methyl-2-(4-methylpyridin-2-yl)propanenitrile

2-Fluoro-4-methylpyridine (1.0 g, 9 mmol), KH(Si(CH₃)₃)₂, (27 mL, 13.5 mmol, 0.5 M solution in toluene) and 2-methylpropanenitrile (3.2 mL) were refluxed in toluene for 1 h. The reaction was quenched with saturated NH₄Cl, the organics separated, dried (MgSO₄), concentrated in-vacuo and the residue purified by column chromatography (20% EtOAc/pet ether) to give 2-methyl-2-(4-methylpyridin-2-yl)propanenitrile (1.22 g, 92%). MS m/z 161.4 (calc'd for C10H₁₂N₂).

### Preparation of 2-(2-cyanopropan-2-yl)isonicotinic acid

2-Methyl-2-(4-methylpyridin-2-yl)propanenitrile (1.22 g, 7.6 mmol) and KMnO₄ (6 g, 38 mmol) were refluxed in H₂O (20 mL) for 3 h. The reaction was cooled, filtered through celite, made pH 4 (1 M HCl) and extracted with EtOAc. The combined organics were dried (MgSO₄), and concentrated in-vacuo to give 2-(2-cyanopropan-2-yl)isonicotinic acid, (550 mg, 38%). MS m/z 191.5 (calc'd for C10H₁₂N₂O₂). The product was used without further purification.

### 1(h) Synthesis of 3,5-bis(1-(tert-butylamino)-2-methyl-1-oxopropan-2-yl)benzoic acid

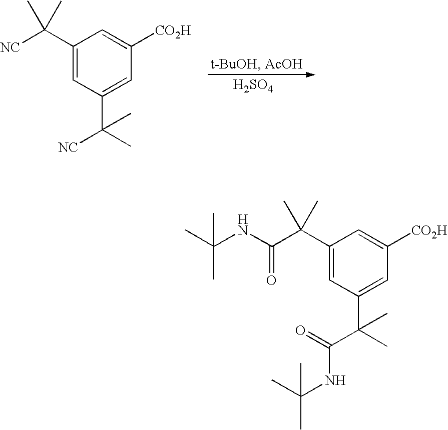

3,5-Bis(2-cyanopropan-2-yl)benzoic acid (0.8 g, 3.1 mmol), t-BuOH (2 mL), AcOH (2 mL) and conc. H₂SO₄ (0.5 mL) were heated at 75° C. for 18 hrs. The crude reaction mixture was diluted with EtOAc (10 mL) and washed with H₂O (3×10 mL). The organic layer was dried (Na₂SO₄), filtered and concentrated in-vacuo to give 3,5-bis(1-(tert-butylamino)-2-methyl-1-oxopropan-2-yl)benzoic acid (0.81 g, 64%). The product was used without further purification. MS m/z [M+H] 403.6 (calc'd for C₂₃H₃₆N₂O₄ 404.5).

### 1(i) Synthesis 3-(1-amino-2-methyl-1-oxopropan-2-yl)benzoic acid

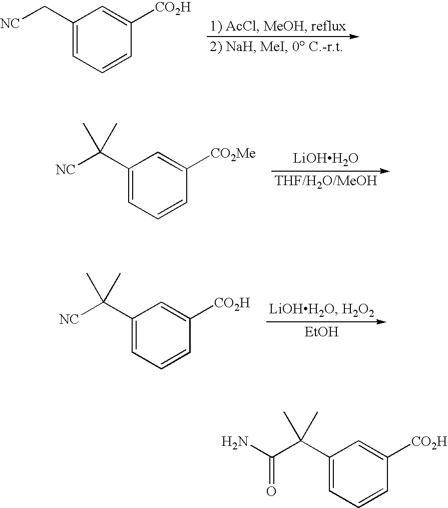

### Preparation of methyl 3-(2-cyanopropan-2-yl)benzoate

3-(1-cyanoethyl)benzoic acid (10.0 g, 57 mmol) and AcCl (8 mL, 114 mmol) were heated at reflux in MeOH (200 mL) for 18 h. The reaction was concentrated in-vacuo, diluted with EtOAc and washed with NaHCO₃ saturated solution. The organics were dried (Na₂SO₄), filtered and concentrated in-vacuo. The crude material was stirred in DMF (250 mL) at 0° C., NaH (3.4 g, 85 mmol) added and the reaction stirred for 30 min at 0° C. MeI (5.3 mL, 85 mmol) was added and the reaction stirred at rt for 18 h. The reaction was cooled (0° C.), quenched with NH₄Cl saturated solution and extracted with EtOAc (3×30 mL). The organics were dried (Na₂SO₄), filtered and concentrated in-vacuo to give methyl 3-(2-cyanopropan-2-yl)benzoate (10.0 g, 86%) as a light brown oil. The product was carried to the next step without further purification.

### Preparation of 3-(2-cyanopropan-2-yl)benzoic acid

Methyl 3-(2-cyanopropan-2-yl)benzoate (1.0 g, 4.9 mmol) and LiOH.H₂O (4 g, 9.8 mmol) were stirred in THF/H₂O/MeOH (3/1/1) (15 mL) at rt for 18 h. The reaction was concentrated in-vacuo and the resultant solution acidified (to pH 2) with conc. HCl. The product was recovered by filtration to give 3-(2-cyanopropan-2-yl)benzoic acid (quantitative) as a yellow solid. MS: (negative ion mode) m/z=188.3 (calcd. for C₁₁H₁₁NO₂ 189.2). The product was used without additional purification.

### Preparation of 3-(1-amino-2-methyl-1-oxopropan-2-yl)benzoic acid

3-(2-cyanopropan-2-yl)benzoic acid (0.5 g, 2.7 mmol), LiOH.H₂O (0.12 g, 2.9 mmol) and H₂O₂ (30% solution) (5 mL) were heated in EtOH (10 mL) at reflux for 18 h. The reaction was concentrated in-vacuo and acidified (to pH 2) with conc. HCl. The resultant residue was diluted with H₂O and extracted sequentially with CH₂Cl₂ (2×10) followed by EtOAc (2×10). The combined organics were dried (Na₂SO₄), filtered and concentrated to give 3-(1-amino-2-methyl-1-oxopropan-2-yl)benzoic acid (0.26 g, 48%) as a white solid. MS: m/z 208.4 (calcd. for C₁₁H₁₃NO₃ 207.2).

### 1(j) Synthesis of 4-(1-(tert-butylamino)-2-methyl-1-oxopropan-2-yl)benzoic acid

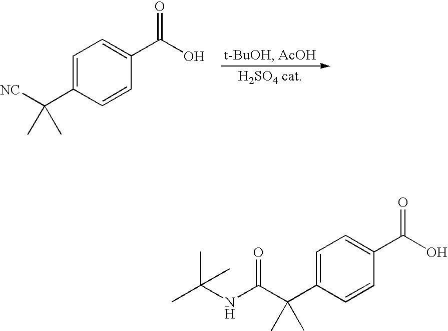

4-(2-Cyanopropan-2-yl)benzoic acid (0.1 g, 0.53 mmol) in t-BuOH (2 mL), AcOH (2 mL) and H₂SO₄ (0.5 mL) were heated at 75° C. for 6 h. The reaction was diluted with H₂O (10 mL), extracted with EtOAc (3×5 mL), dried (Na₂SO₄) and concentrated in-vacuo to give 4-(1-(t-butylamino)-2-methyl-1-oxopropan-2-yl)benzoic acid (0.12 g, 86%). MS: m/z=264.3 (calcd. for C15H₂₁NO₃ 263.3). The product was used without additional purification.

### Example 2

### Procedures for the Synthesis of Compounds with Generic Structure

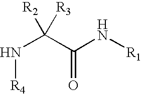

### R₂R₃=Cy-Propyl, Cy-Pentyl, Cy-Hexyl, C4-THP, C4-N-subs-pip, C3-Indanyl R₄=ArCO—, ARSO₂—

### Method A: Exemplified by Synthesis of 3,5-di-tert-butyl-4-hydroxy-N-(1-(thiazol-2-ylmethylcarbamoyl)cyclopropyl)benzamide (Compound 1)

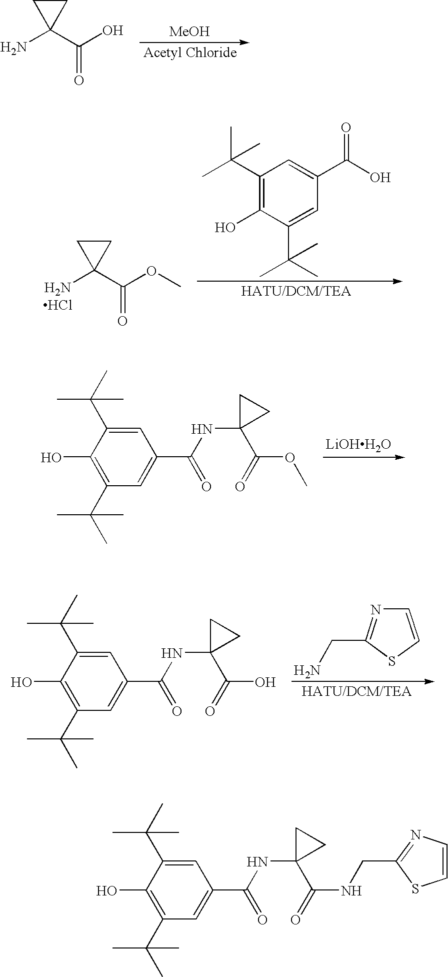

### Preparation of methyl 1-aminocyclopropanecarboxylate hydrochloride

Acetyl chloride (10 mL) was added dropwise with stirring to MeOH (10 mL). The resultant solution was added dropwise to a suspension of 1-aminocyclopropanecarboxylic acid, (2.5 g, 24.7 mmol) in MeOH (20 mL) and the reaction refluxed for 16 h. The reaction was cooled and concentrated in-vacuo to give methyl 1-aminocyclopropanecarboxylate hydrochloride (3.77 g, 100%), MS: m/z=116.2 (calcd. for C5H₉NO₂ 115.1). The product was used without further purification.

### Preparation of methyl 1-(3,5-di-tert-butyl-4-hydroxybenzamido)cyclopropanecarboxylate

Methyl 1-aminocyclopropanecarboxylate hydrochloride (1.5 g, 10 mmol), 3,5-di-tert-butyl-4-hydroxybenzoic acid (2.5 g, 10 mmol), HATU (5.55 g, 15 mmol) and TEA (7 mL, 50 mmol) were stirred in DCM (25 mL) at rt for 48 h. The resultant precipitate was collected by filtration to give methyl 1-(3,5-di-tert-butyl-4-hydroxybenzamido)cyclopropanecarboxylate (2.0 g, 57.6%). MS: m/z=348.6 (calcd. for C20H₂₉NO₄ 347.5). The product was used without further purification.

### Preparation of 1-(3,5-di-tert-butyl-4-hydroxybenzamido)cyclopropanecarboxylic acid

Methyl 1-(3,5-di-tert-butyl-4-hydroxybenzamido)cyclopropanecarboxylate (2 g, 5.8 mmol) and LiOH.H₂O (727 mg, 17.3 mmol) were stirred in THF:H₂O (20 mL, 1:1) until hydrolysis was complete. The THF was removed in-vacuo and the aqueous layer acidified (1 M HCl). The precipitate was recovered by filtration to give 1-(3,5-di-tert-butyl-4-hydroxybenzamido)cyclopropanecarboxylic acid (1.58 g, 82.3%). MS: (negative ion mode) m/z=332.6 (calcd. for C₁₉H₂₅NO₄ 333.4). The product was used without further purification.

### Preparation of 3,5-di-tert-butyl-4-hydroxy-N-(1-(thiazol-2-ylmethylcarbamoyl)cyclopropyl)-benzamide

1-(3,5-Di-tert-butyl-4-hydroxybenzamido)cyclopropanecarboxylic acid (100 mg, 0.3 mmol), thiazol-2-ylmethanamine (50 mg, 0.45 mmol), HATU (171 mg, 0.45 mmol) and DIPEA (0.15 mL, 0.9 mmol) were stirred in DCM at rt for 16 h. The organics were washed sequentially with saturated NaHCO₃, 0.1 M HCl, dried (Na₂SO₄), concentrated in-vacuo and the residue purified by Biotage (5% MeOH/DCM) to give 3,5-di-tert-butyl-4-hydroxy-N-(1-(thiazol-2-ylmethylcarbamoyl)cyclopropyl)-benzamide (57 mg, 45.0%). MS: m/z=430.2 (calc'd for C₂₃H₃₁N₃O₃S 429.2).

### Method B: Exemplified by the synthesis of 3,5-di-tert-butyl-4-hydroxy-N-(1-(pyridin-2-ylcarbamoyl)cyclopropyl)benzamide (Compound 4)

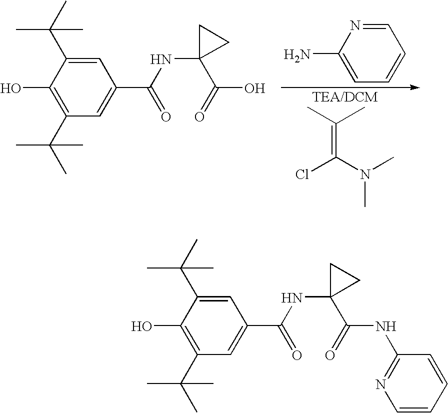

1-(3,5-Di-tert-butyl-4-hydroxybenzamido)cyclopropanecarboxylic acid (100 mg, 0.3 mmol) and 1-chloro-N,N,2-trimethylprop-1-en-1-amine (52 mg, 0.4 mmol) were stirred in DCM (5 mL) at rt for 10 min. 2-Aminopyridine (50 mg, 0.45 mmol) and TEA (61 mg, 0.6 mmol) were added and the reaction stirred at rt for 2 h. The reaction was partitioned between DCM and H₂O, the organics separated, dried (Na₂SO₄), concentrated in-vacuo and the residue purified by Biotage (5% MeOH/DCM) to give 3,5-di-tert-butyl-4-hydroxy-N-(1-(pyridin-2-ylcarbamoyl)cyclopropyl)benzamide. (64 mg, 52.0%). MS m/z 410.5 (calc'd for C₂₄H₃₁N₃O₃ 409.2).

### Method C: Exemplified by the synthesis of 3,5-di-tert-butyl-4-hydroxy-N-(1-((5-methylpyrazin-2-yl)methylcarbamoyl)cyclohexyl)benzamide (Compound 5)

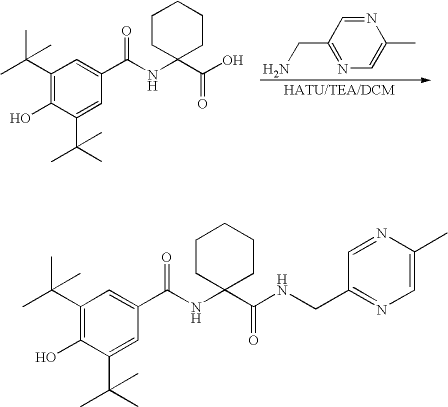

### 1-(3,5-Di-tert-butyl-4-hydroxybenzamido)cyclohexanecarboxylic acid was prepared in an analogous fashion to intermediate

### Preparation of 3,5-di-tert-butyl-4-hydroxy-N-(1-((5-methylpyrazin-2-yl)methylcarbamoyl)-cyclohexyl)benzamide

1-(3,5-Di-tert-butyl-4-hydroxybenzamido)cyclohexanecarboxylic acid (200 mg, 0.53 mmol), 2-(aminomethyl)-5-methylpyrazine (79 mg, 0.64 mmol), HATU (263 mg, 0.69 mmol) and TEA (0.3 mL, 2.13 mmol) were stirred in DCM (5 mL) at rt for 48 h. Silica bound isocyanate resin (0.53 mmol, 1 eq) and silica bound carbonate resin (0.53 mmol, 1 eq) were added and left for 20 h. The resin was removed by filtration, washed with DCM (4 mL), the filtrate concentrated in-vacuo and the residue purified by Biotage (5% MeOH/DCM followed by 1% MeOH/1% TEA/EtOAc) to give 3,5-di-tert-butyl-4-hydroxy-N-(1-((5-methylpyrazin-2-yl)methylcarbamoyl)-cyclohexyl)benzamide (26 mg, 10.2%). MS: m/z=481.5 (calcd. for C₂₈H₄₀N₄O₃ 480.6).

### Method D: Exemplified by the synthesis of 2,6-di-tert-butyl-N-(1-(thiazol-2-ylmethylcarbamoyl)cyclopropyl)isonicotinamide (Compound 10)

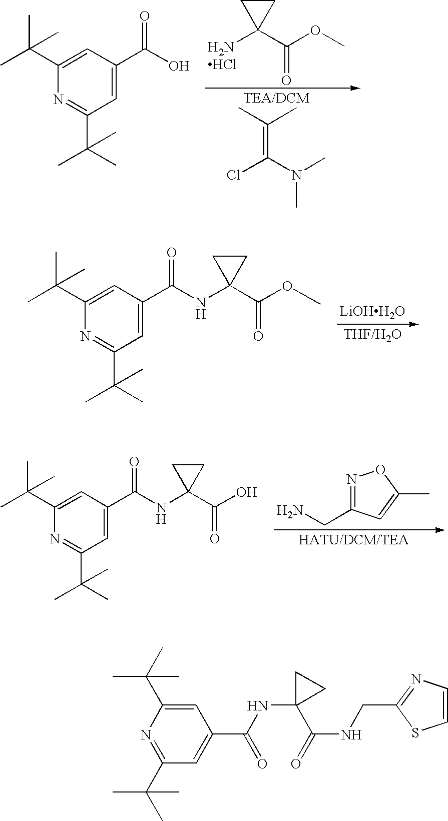

### Preparation of methyl 1-(2,6-di-tert-butylisonicotinamido)cyclopropanecarboxylate (47)

This compound was prepared in an analogous fashion to 3,5-di-tert-butyl-4-hydroxy-N-(1-(pyridin-2-ylcarbamoyl)cyclopropyl)benzamide, method B.

### Preparation of 2,6-di-tert-butyl-N-(1-(thiazol-2-ylmethylcarbamoyl)cyclopropyl)isonicotinamide

The hydrolysis and coupling were conducted in an analogous fashion to 3,5-di-tert-butyl-4-hydroxy-N-(1-(thiazol-2-ylmethylcarbamoyl)cyclopropyl)benzamide (39), Method A. (63 mg, 48%), MS: m/z=417 (calcd. for C₂₂H₃₀N₄O₂S 414.6).

### Method E: Exemplified by the synthesis of N-benzyl-1-(2-(3,5-di-tert-butyl-4-methoxyphenyl)acetamido)cyclohexanecarboxamide (Compound 17)

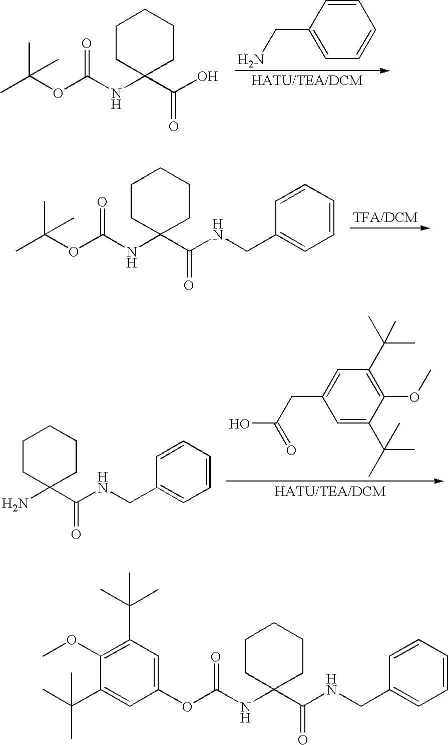

### Preparation of t-butyl 1-(benzylcarbamoyl)cyclohexylcarbamate

1-(t-Butoxycarbonylamino)cyclohexanecarboxylic acid (10 g, 41.2 mmol), Benzylamine (4.5 mL, 41.2 mmol), HATU (22.9 g, 61.8 mmol) and TEA (17.3 mL, 123.6 mmol) were stirred in DCM (150 mL) at rt for 16 h. The reaction was diluted with DCM (250 mL) washed with 1 M HCl and the resultant PPT removed by filtration. The filtrate was washed with saturated NaHCO₃, dried (MgSO₄), concentrated in-vacuo and the residue taken up in the minimum amount of DCM. Et₂O was added to PPT and the suspension stirred at rt for 1 h. The product was collected by filtration to give t-butyl 1-(benzylcarbamoyl)cyclohexylcarbamate (6.57 g, 48%). MS: m/z 333.7 (calcd. for C₁₉H₁₈N₂O₃ 414.6). The product was used without further purification.

### Preparation of 1-amino-N-benzylcyclohexanecarboxamide

t-Butyl 1-(benzylcarbamoyl)cyclohexylcarbamate (7.47 g, 22.5 mmol) was stirred in DCM/TFA (120 mL, 5:1) at rt for 16 h. The reaction was concentrated in-vacuo, washed with 10% NaOH solution, dried (MgSO₄) and concentrated in-vacuo to give 1-amino-N-benzylcyclohexanecarboxamide (5.04 g, 97%). MS: m/z 233.5 (calcd. for C₁₉H₁₈N₂O₃ 414.6).

### Preparation of N-benzyl-1-(2-(3,5-di-tert-butyl-4-methoxyphenyl)acetamido)cyclohexane-carboxamide

The coupling was conducted in an analogous fashion to 3,5-di-tert-butyl-4-hydroxy-N-(1-(thiazol-2-ylmethylcarbamoyl)cyclopropyl)benzamide, Method A. (108 mg, 54%), MS: m/z M+Na=515.8 (calcd. for C₃₁H₄₄N₂O₃ 492.7).

### Method F exemplified by the synthesis of 4-(4-((dimethylamino)methyl)benzylcarbamoyl)-tetrahydro-2H-pyran-4-yl 2,6-di-tert-butylisonicotinate (Compound 23)

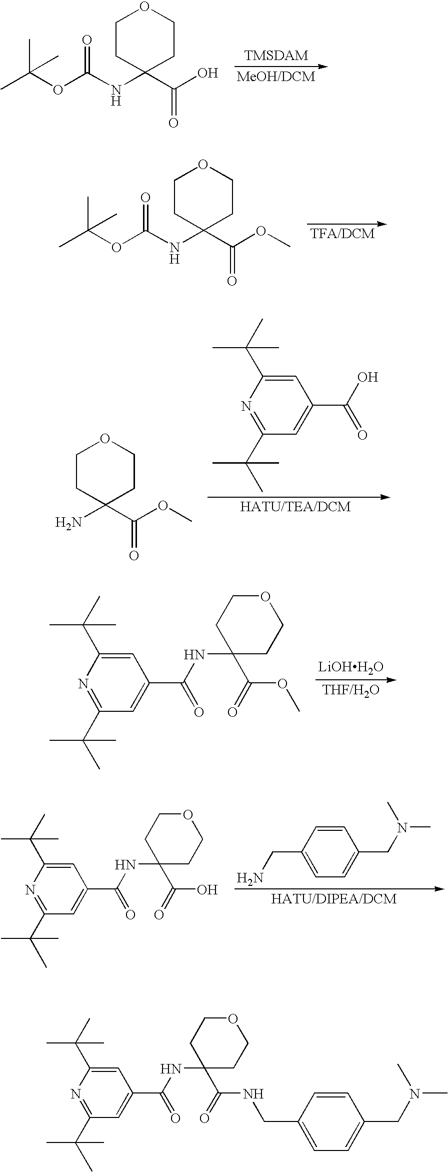

### Preparation of methyl 1-(tert-butoxycarbonylamino)cyclohexanecarboxylate

1-(t-Butoxycarbonylamino)cyclohexanecarboxylic acid (2.5 g, 10.3 mmol) was stirred as a suspension in DCM:MeOH (25 mL, 4:1) at rt and TMSDAM (2 M solution in hexanes (6.5 mL, 13 mmol) was added dropwise. Excess reagent was decomposed with the dropwise addition of AcOH. The reaction was concentrated in-vacuo and the residue purified by Biotage (20% EtOAc/pet ether) to give methyl 1-(tert-butoxycarbonylamino)cyclohexanecarboxylate (2.68 g, 100%).

### Preparation of methyl 1-aminocyclohexanecarboxylate

The compound was prepared in an analogous manner to 1-amino-N-benzylcyclohexanecarboxamide Method E

### Preparation of 4-(4-((dimethylamino)methyl)benzylcarbamoyl)-tetrahydro-2H-pyran-4-yl 2,6-di-tert-butylisonicotinate

The compound was prepared using the analogous coupling, hydrolysis, coupling protocol for the synthesis of 3,5-di-tert-butyl-4-hydroxy-N-(1-(thiazol-2-ylmethylcarbamoyl)cyclopropyl)-benzamide (Method A) (19 mg, 17.5%). %). MS m/z 509.9 (calc'd for C30H₄₄N₄O₃ 508.3).

### Method G exemplified by the synthesis of N-benzyl-1-(3,5-di-tert-butylbenzylamino)cyclohexanecarboxamide (Compound 57)

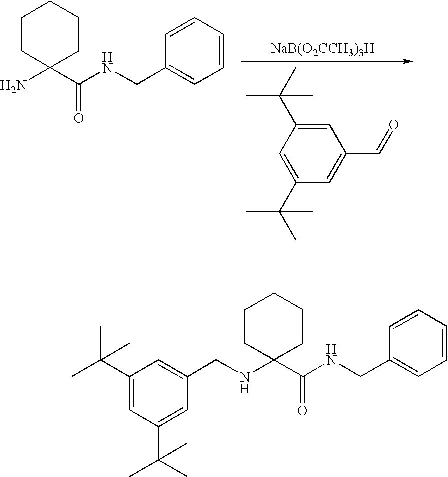

1-Amino-N-benzylcyclohexanecarboxamide (180 mg, 0.76 mmol), 3,5-di-tert-butylbenzaldehyde (200 mg, 0.93 mmol) and sodium triacetoxy borohydride (330 mg, 1.55 mmol) were stirred in DCM at rt for 16 h. The organics were washed with H₂O, dried (Na₂SO₄), concentrated in-vacuo and the product isolated by mass directed RPLC.

### Method H as exemplified by the synthesis of 3,5-di-tert-butyl-N-(1-(3-(trifluoromethyl)benzylcarbamoyl)cyclohexyl)benzamide (Compound 62)

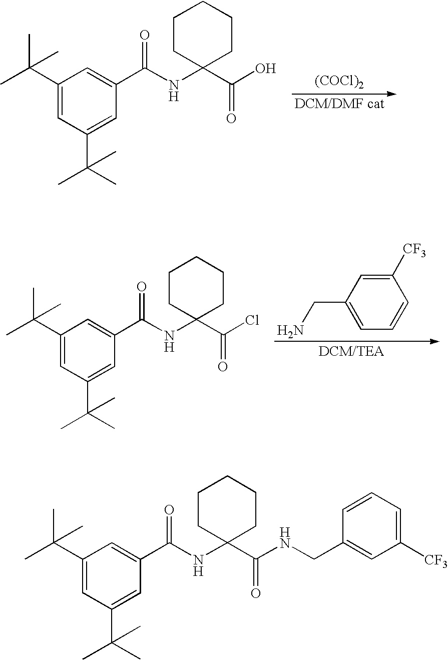

1-(3,5-Di-tert-butylbenzamido)cyclohexanecarboxylic acid was prepared in an analogous fashion to 1-(3,5-di-tert-butyl-4-hydroxybenzamido)cyclopropanecarboxylic acid (Method A)

### Preparation of 1-(3,5-di-tert-butylbenzamido)cyclohexanecarbonyl chloride

1-(3,5-Di-tert-butylbenzamido)cyclohexanecarboxylic acid (150 mg, 0.42 mmol) and oxalyl chloride (0.4 mL, 4.2 mmol) were stirred in DCM (5 mL) at rt. A catalytic amount of DMF was added and the reaction stirred at rt for 16 h. The reaction was concentrated in-vacuo and the crude 1-(3,5-di-tert-butylbenzamido)cyclohexanecarbonyl chloride was used without subsequent purification.

### Preparation of 3,5-di-tert-butyl-N-(1-(3-(trifluoromethyl)benzylcarbamoyl)cyclohexyl)-benzamide

Crude 1-(3,5-di-tert-butylbenzamido)cyclohexanecarbonyl chloride (0.42 mmol), (3-(trifluoromethyl)phenyl)methanamine (75 μL, 0.5 mmol) and TEA (0.11 mL, 0.33 mmol) were stirred in DCM at rt for 24 h. The reaction was washed with H₂O, dried (Na₂SO₄), concentrated in-vacuo and the residue purified by Biotage (5% EtOAc/DCM) to give 3,5-di-tert-butyl-N-(1-(3-(trifluoromethyl)benzylcarbamoyl)cyclohexyl)-benzamide. The product was isolated by mass-directed RPLC.

### Method I as exemplified by the synthesis of N-(4-(benzylcarbamoyl)-1-methylpiperidin-4-yl)-2,6-di-tert-butylisonicotinamide (Compound 97)

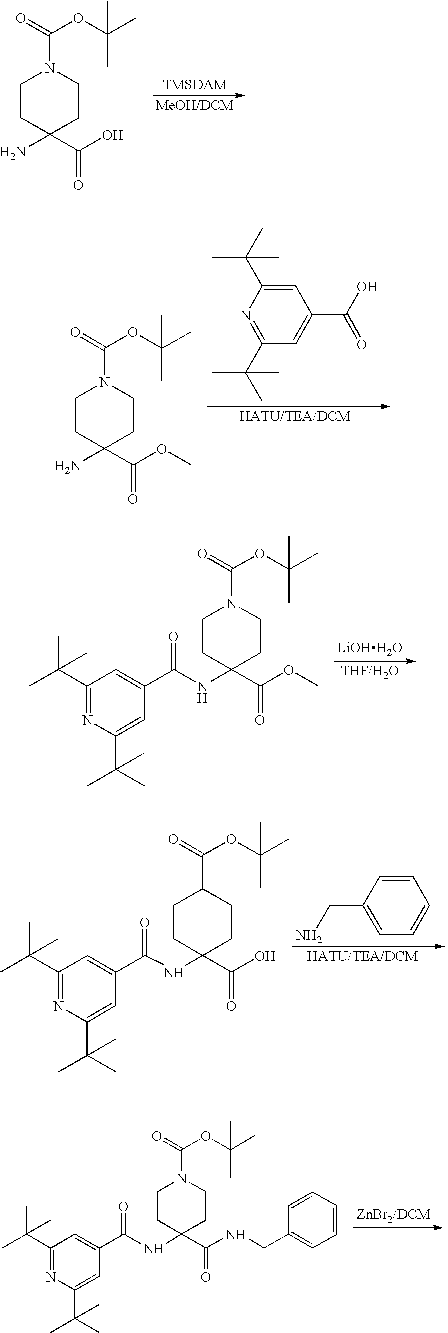

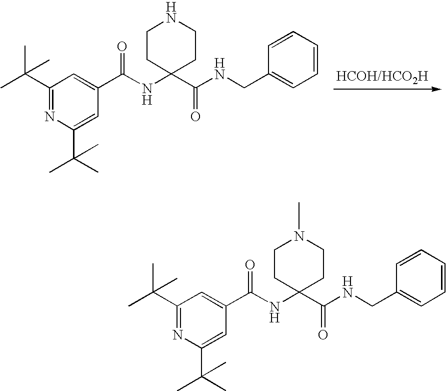

### Preparation of 1-t-Butyl 4-methyl 4-aminopiperidine-1,4-dicarboxylate

1-t-Butyl 4-methyl 4-aminopiperidine-1,4-dicarboxylate was prepared in an analogous manner to methyl 1-(tert-butoxycarbonylamino)cyclohexanecarboxylate (Method F) using MeOH (100%) as solvent.

### Preparation of t-butyl 4-(benzylcarbamoyl)-4-(2,6-di-tert-butylisonicotinamido)piperidine-1-carboxylate

The compound was prepared using the analogous coupling, hydrolysis, coupling protocol for the synthesis of 3,5-di-tert-butyl-4-hydroxy-N-(1-(thiazol-2-ylmethylcarbamoyl)cyclopropyl)-benzamide (Method A) (226.5 mg, 41.0%). MS: m/z 551.7 (calcd. for C₃₆H₄₆N₄O₄ 550.7).

### Preparation of N-(4-(benzylcarbamoyl)piperidin-4-yl)-2,6-di-tert-butylisonicotinamide

t-Butyl 4-(benzylamino)-4-(2,6-di-tert-butylpyridin-4-ylcarbamoyl)piperidine-1-carboxylate (0.20 g, 0.364 mmol) and ZnBr₂ (0.245 g, 1.09 mmol) were stirred in DCM (60 mL) at rt for 72 h. H₂O (3 mL) was added and the DCM solution washed with brine (30 mL), dried (Na₂SO₄), concentrated in-vacuo and the residue purified by column chromatography (DCM:MeOH:NH₄OH, 100:5:1) to give N-(4-(benzylcarbamoyl)piperidin-4-yl)-2,6-di-tert-butylisonicotinamide (0.12 g, 73.2%) MS m/z 423.5 (calcd C₂₆H₃₈N₄O, 422.30).

### Preparation of N-(4-(benzylcarbamoyl)-1-methylpiperidin-4-yl)-2,6-di-tert-butylisonicotinamide

N-(4-(benzylcarbamoyl)piperidin-4-yl)-2,6-di-tert-butylisonicotinamide (0.10 g, 0.22 mmol), fomic acid (1 mL) and formaldehyde 37% solution in H₂O (0.45 mL) were heated at 50° C. for 2 h. The solution was made pH 10 with 2M NaOH (dropwise) and extracted with DCM (20 mL). The organics were washed with brine (30 mL), dried (Na₂SO₄), concentrated in-vacuo and the residue purified by column chromatography (DCM:MeOH:NH₄OH, 100:5:1) to give N-(4-(benzylcarbamoyl)-1-methylpiperidin-4-yl)-2,6-di-tert-butylisonicotinamide (0.50 g, 48.4%) MS m/z 436.8 (calcd C₂₇H₄₀N₄O, 436.63).

### Method J as exemplified by the synthesis of 3,5-di-tert-butyl-4-methoxy-N-(1-(phenylcarbamoyl)cyclohexyl)benzamide (Compound 96)

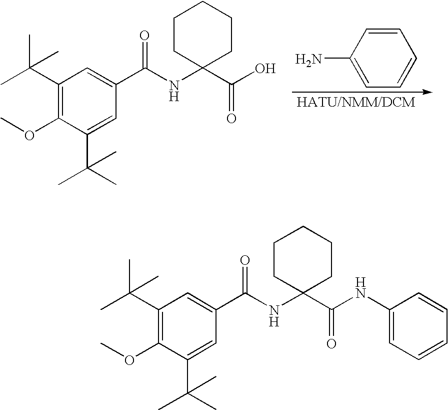

1-(3,5-di-t-Butyl-4-methoxybenzamido)cyclohexanecarboxylic acid was prepared in an analogous manner to 1-(3,5-di-tert-butyl-4-hydroxybenzamido)cyclopropanecarboxylic acid (method A).

### Preparation of 3,5-di-tert-butyl-4-methoxy-N-(1-(phenylcarbamoyl)cyclohexyl)benzamide

1-(3,5-di-t-Butyl-4-methoxybenzamido)cyclohexanecarboxylic acid (19.5 mg, 0.05 mmol), aniline (9.1 μL, 0.1 mmol), HATU (27 mg, 0.7 mmol) and NMM (16 μL, 0.15 mmol) were heated in DCM (0.5 mL) at 120° C. for 30 mins using microwave assisted heating. Silica bound carbonate (1 eq) and silica bound isocyanate (1 eq) were added and the suspension allowed to stand for 48 h. The resin was removed by filtration, washed with DCM (4 mL) and the organics concentrated in-vacuo. The residue was purified by biotage (3% EtOAc/DCM) to give 1-(3,5-di-t-Butyl-4-methoxybenzamido)cyclohexanecarboxylic acid (17 mg, 37%). MS m/z 465.2 (calcd C₂₇H₄₀N₄O, 464.3).

### Example 3

### Synthesized Compounds

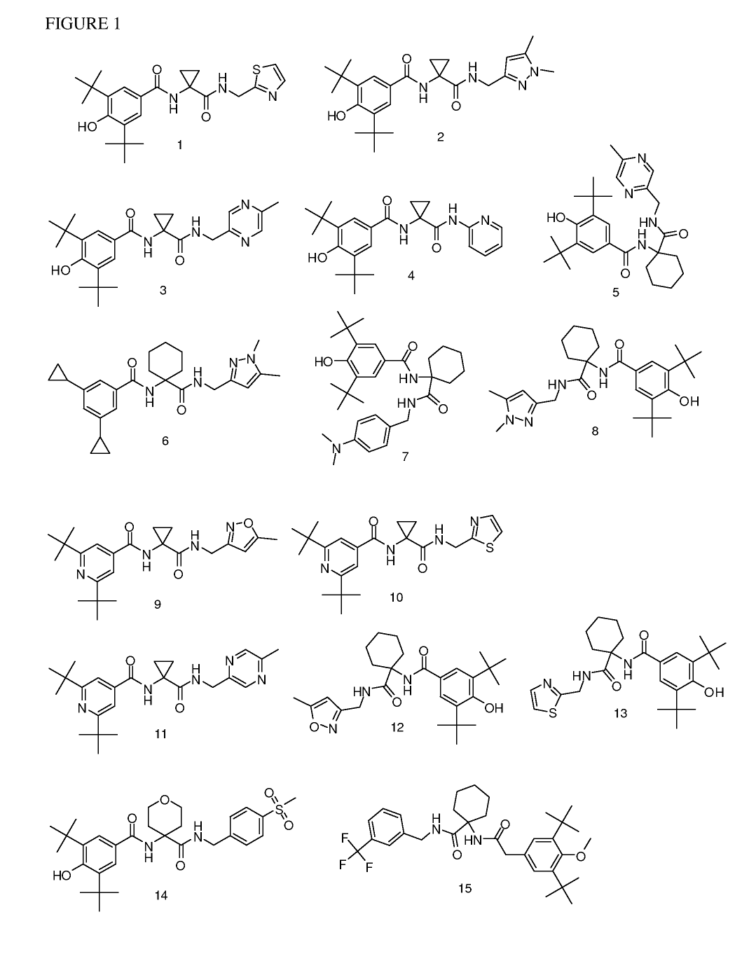

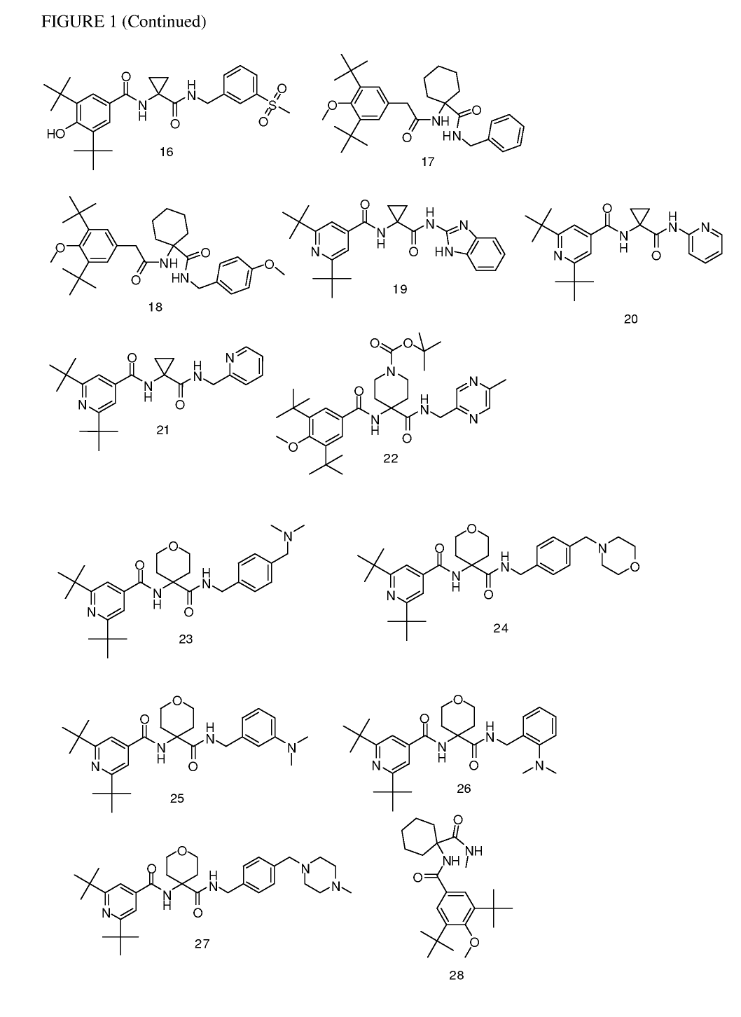

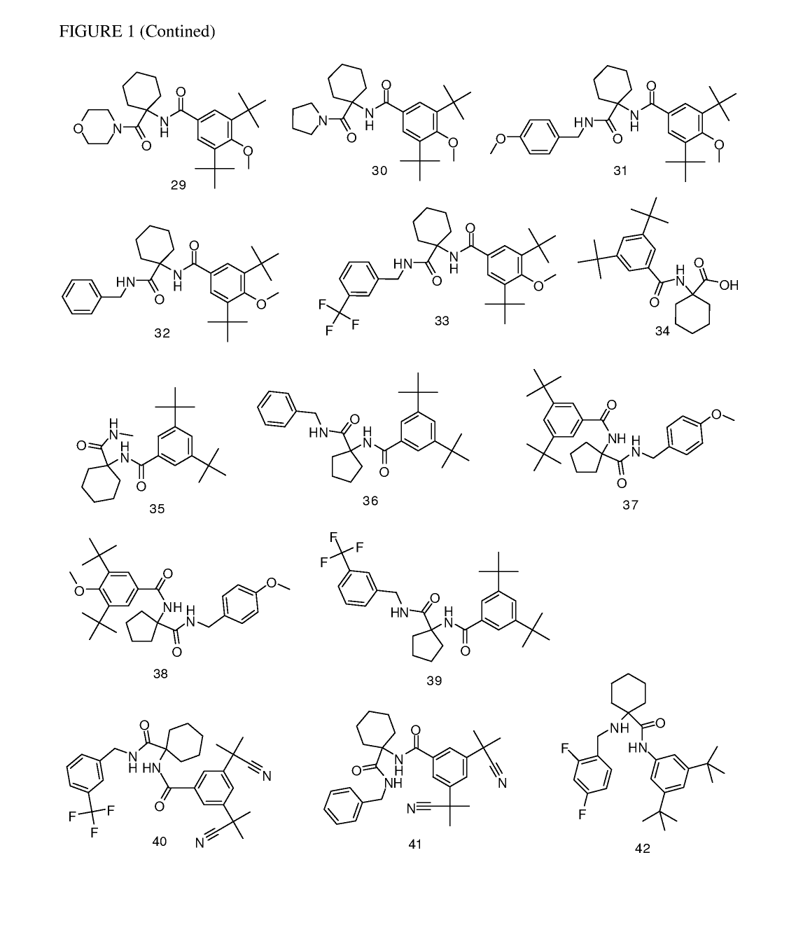

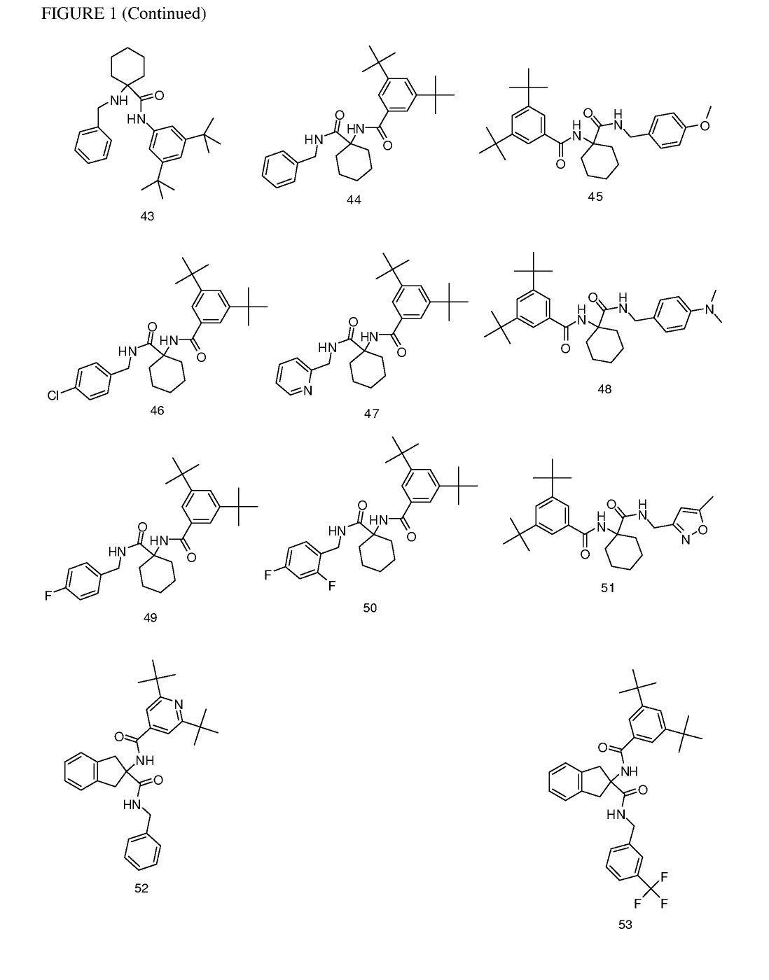

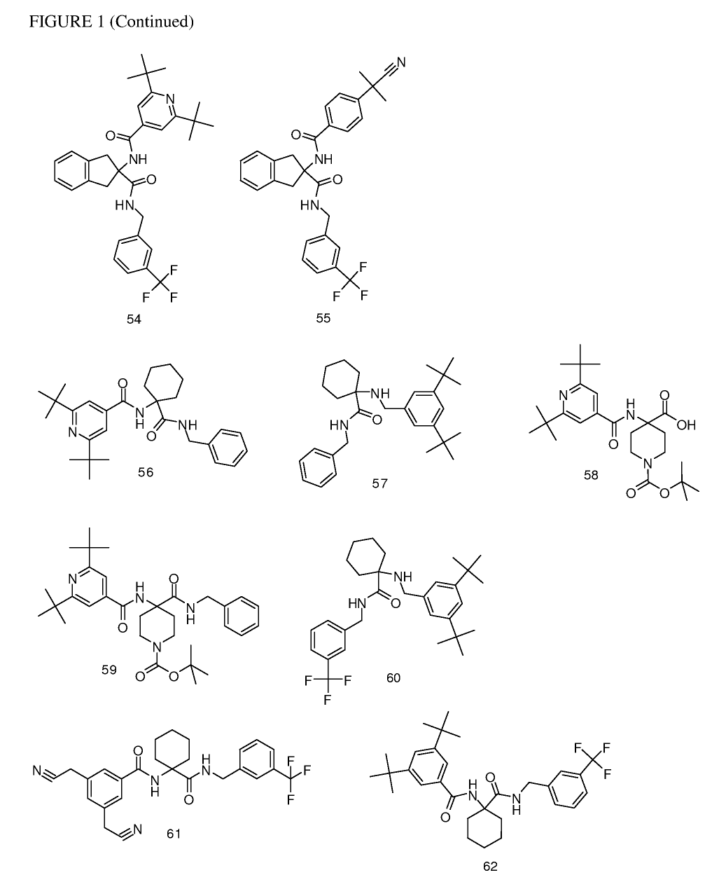

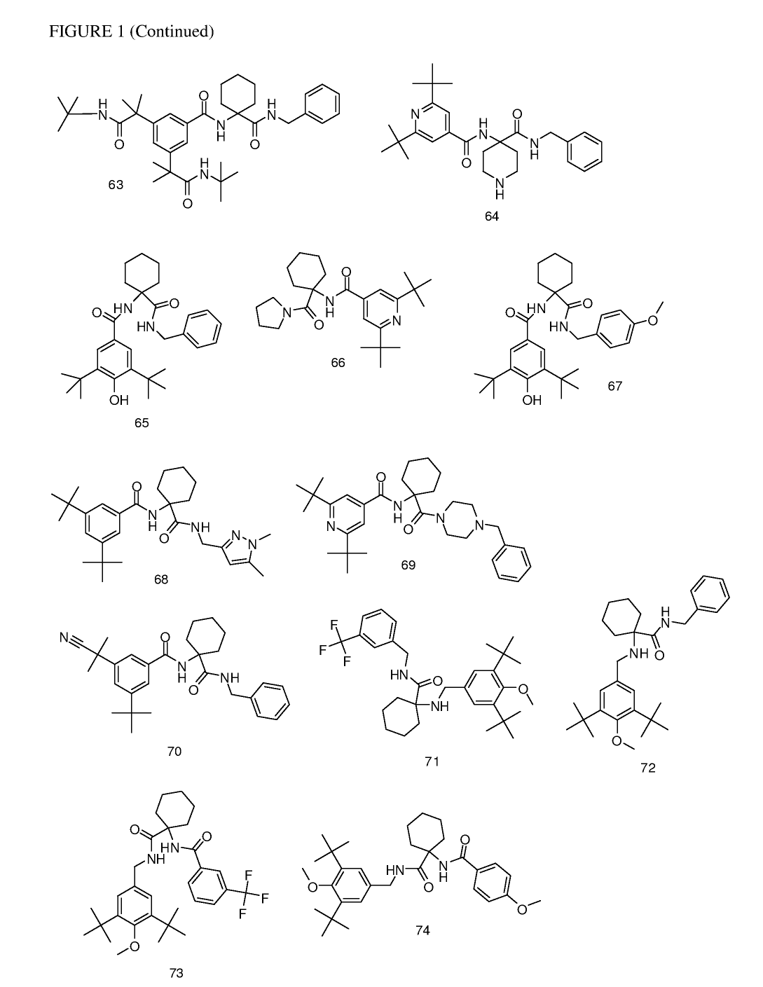

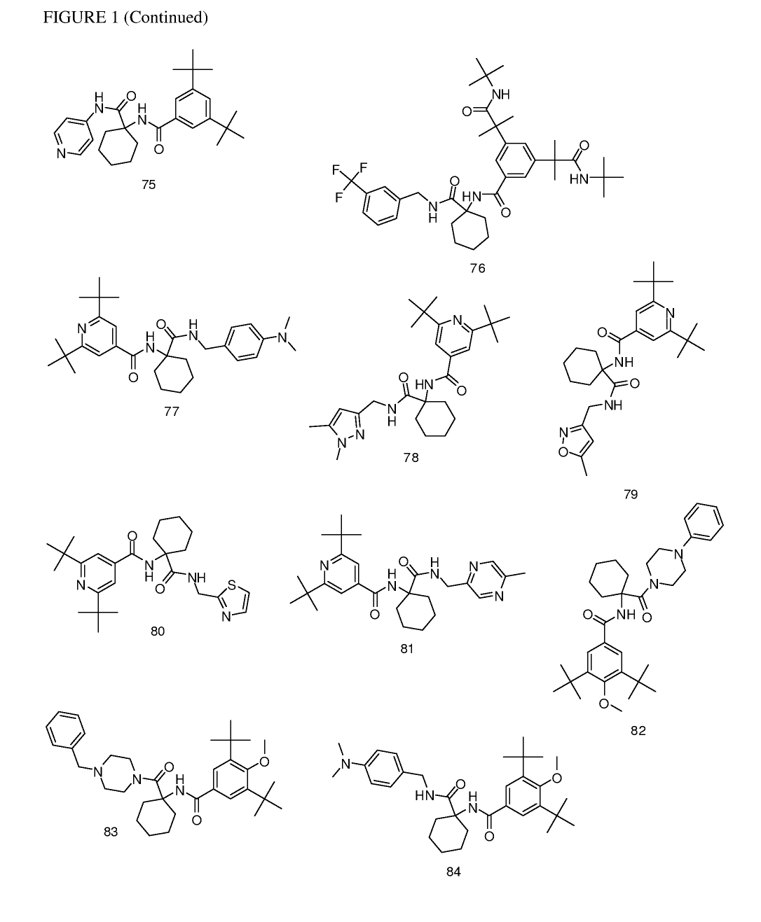

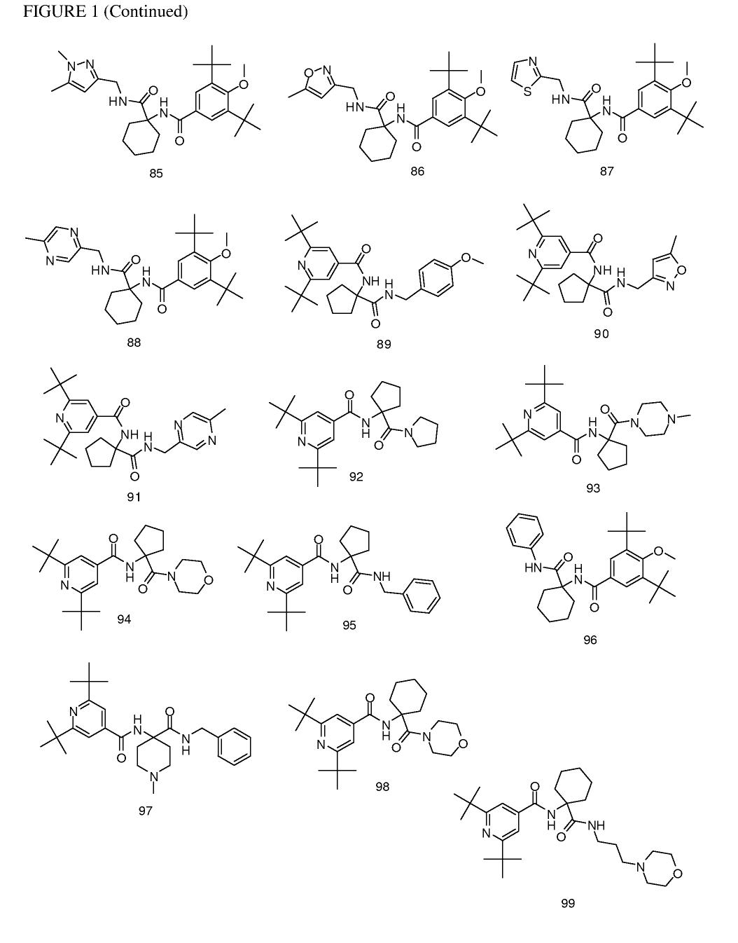

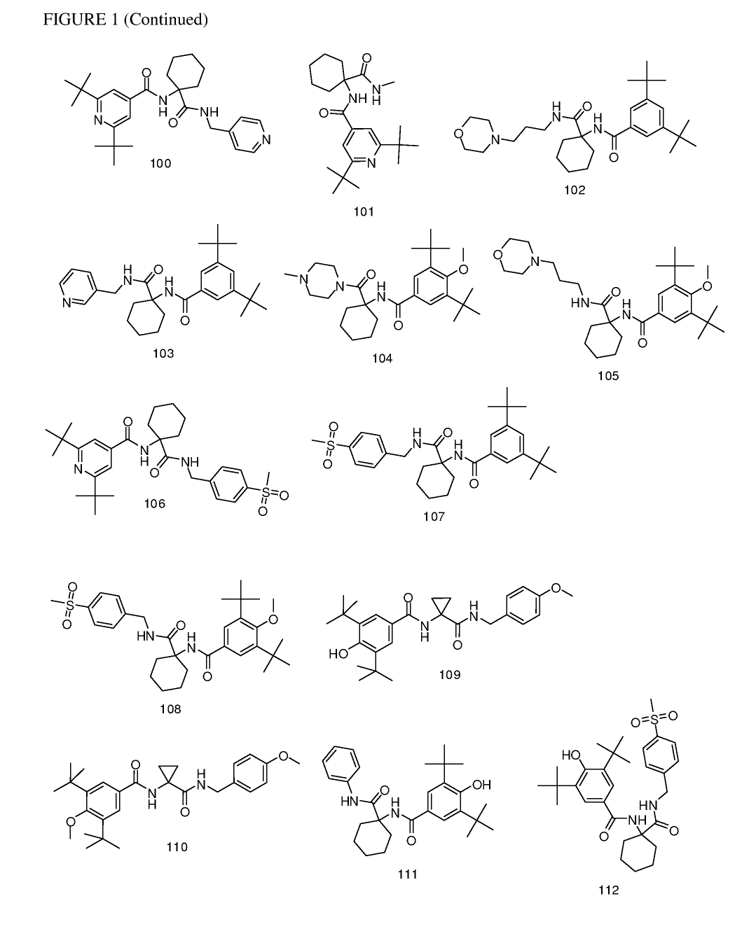

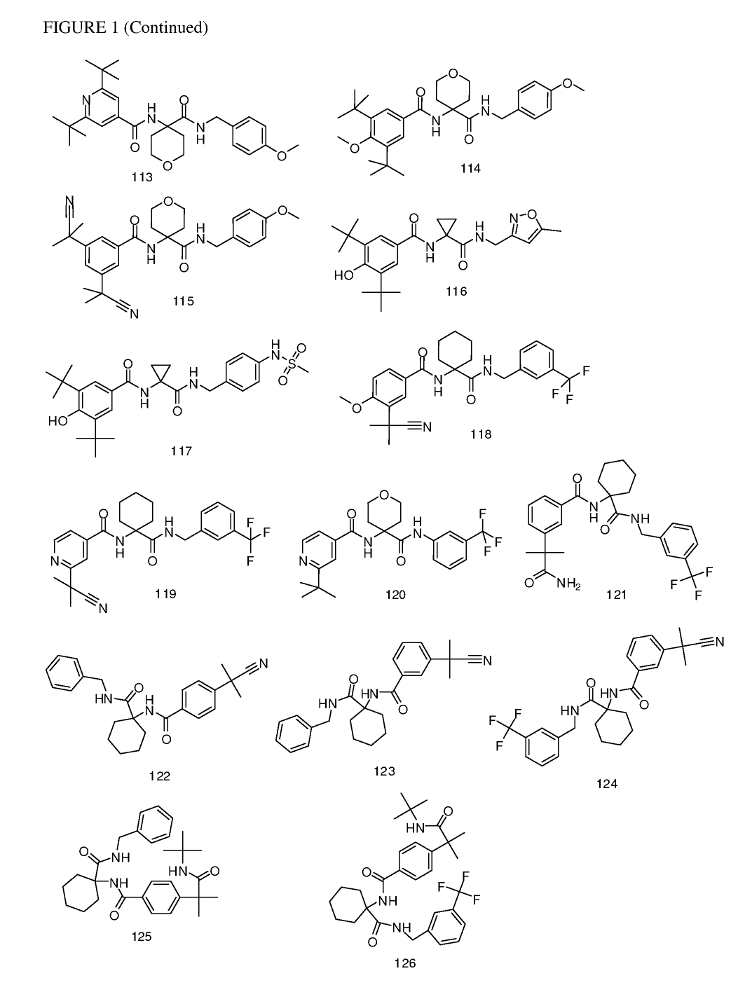

Following the general procedures set forth above, the following compounds listed in Table 1 below were prepared. The corresponding structures are illustrated in FIG. 1.

TABLE 1

| Cmpd | | Method of | Observed |
|---|---|---|---|
| No. | Compound | synthesis | [M + H]+ |
| 1 | 3,5-di-tert-butyl-4-hydroxy-N-(1-(thiazol-2- | A | 430.3 |
| | ylmethylcarbamoyl)cyclopropyl)benzamide | | |
| 2 | 3,5-di-tert-butyl-N-(1-((1,5-dimethyl-1H-pyrazol-3- | A | 441.4 |
| | yl)methylcarbamoyl)cyclopropyl)-4-hydroxybenzamide | | |
| 3 | 3,5-di-tert-butyl-4-hydroxy-N-(1-((5-methylpyrazin-2- | A | 439.5 |
| | yl)methylcarbamoyl)cyclopropyl)benzamide | | |
| 4 | 3,5-di-tert-butyl-4-hydroxy-N-(1-(pyridin-2- | B | 410.5 |
| | ylcarbamoyl)cyclopropyl)benzamide | | |
| 5 | 3,5-di-tert-butyl-4-hydroxy-N-(1-((5-methylpyrazin-2- | C | 479.3 |
| | yl)methylcarbamoyl)cyclohexyl)benzamide | | |
| 6 | 3,5-dicyclopropyl-N-(1-((1,5-dimethyl-1H-pyrazol-3- | E | 435 |
| | yl)methylcarbamoyl)cyclohexyl)benzamide | | |
| 7 | 3,5-di-tert-butyl-N-(1-(4- | C | 508.6 |
| | (dimethylamino)benzylcarbamoyl)cyclohexyl)-4- | | |
| | hydroxybenzamide | | |
| 8 | 3,5-di-tert-butyl-N-(1-((1,5-dimethyl-1H-pyrazol-3- | C | 483.6 |
| | yl)methylcarbamoyl)cyclohexyl)-4-hydroxybenzamide | | |
| 9 | 2,6-di-tert-butyl-N-(1-((5-methylisoxazol-3- | D | 413.7 |
| | yl)methylcarbamoyl)cyclopropyl)isonicotinamide | | |
| 10 | 2,6-di-tert-butyl-N-(1-(thiazol-2- | D | 415.7 |
| | ylmethylcarbamoyl)cyclopropyl)isonicotinamide | | |
| 11 | 2,6-di-tert-butyl-N-(1-((5-methylpyrazin-2- | D | 424.8 |
| | yl)methylcarbamoyl)cyclopropyl)isonicotinamide | | |
| 12 | 3,5-di-tert-butyl-4-hydroxy-N-(1-((5-methylisoxazol-3- | C | 470.8 |
| | yl)methylcarbamoyl)cyclohexyl)benzamide | | |
| 13 | 3,5-di-tert-butyl-4-hydroxy-N-(1-(thiazol-2- | C | 472.8 |
| | ylmethylcarbamoyl)cyclohexyl)benzamide | | |
| 14 | 4-(3,5-di-tert-butyl-4-hydroxybenzamido)-N-(4- | E | 545.8 |
| | (methylsulfonyl)benzyl)tetrahydro-2H-pyran-4-carboxamide | | |
| 15 | 1-(2-(3,5-di-tert-butyl-4-methoxyphenyl)acetamido)-N-(3- | E | 561.9 |
| | (trifluoromethyl)benzyl)cyclohexanecarboxamide | | |
| 16 | 3,5-di-tert-butyl-4-hydroxy-N-(1-(3- | D | 501.3 |
| | (methylsulfonyl)benzylcarbamoyl)cyclopropyl)benzamide | | |
| 17 | N-benzyl-1-(2-(3,5-di-tert-butyl-4- | E | 493.8 |
| | methoxyphenyl)acetamido)cyclohexanecarboxamide | | |
| 18 | 1-(2-(3,5-di-tert-butyl-4-methoxyphenyl)acetamido)-N-(4- | E | 523.9 |
| | methoxybenzyl)cyclohexanecarboxamide | | |
| 19 | N-(1-(1H-benzo[d]imidazol-2-ylcarbamoyl)cyclopropyl)-2,6-di-tert- | B | 434.8 |
| | butylisonicotinamide | | |
| 20 | 2,6-di-tert-butyl-N-(1-(pyridin-2- | D | 395.7 |
| | ylcarbamoyl)cyclopropyl)isonicotinamide | | |
| 21 | 2,6-di-tert-butyl-N-(1-(pyridin-2- | D | 409.8 |
| | ylmethylcarbamoyl)cyclopropyl)isonicotinamide | | |
| 22 | tert-butyl 4-(3,5-di-tert-butyl-4-methoxybenzamido)-4-((5- | I(SYNTHETIC | 596.9 |
| | methylpyrazin-2-yl)methylcarbamoyl)piperidine-1-carboxylate | INTERMEDIATE) | |
| 23 | 2,6-di-tert-butyl-N-(4-(4- | F | 509.9 |
| | ((dimethylamino)methyl)benzylcarbamoyl)tetrahydro-2H-pyran-4- | | |
| | yl)isonicotinamide | | |
| 24 | 2,6-di-tert-butyl-N-(4-(4- | F | 551.9 |
| | (morpholinomethyl)benzylcarbamoyl)tetrahydro-2H-pyran-4- | | |
| | yl)isonicotinamide | | |
| 25 | 2,6-di-tert-butyl-N-(4-(3- | F | 495.9 |
| | (dimethylamino)benzylcarbamoyl)tetrahydro-2H-pyran-4- | | |
| | yl)isonicotinamide | | |
| 26 | 2,6-di-tert-butyl-N-(4-(2- | F | 495.9 |
| | (dimethylamino)benzylcarbamoyl)tetrahydro-2H-pyran-4- | | |
| | yl)isonicotinamide | | |
| 27 | 2,6-di-tert-butyl-N-(4-(4-((4-methylpiperazin-1- | F | 564.9 |
| | yl)methyl)benzylcarbamoyl)tetrahydro-2H-pyran-4- | | |
| | yl)isonicotinamide | | |
| 28 | 3,5-di-tert-butyl-4-methoxy-N-(1- | A | 403.1 |
| | (methylcarbamoyl)cyclohexyl)benzamide | | |
| 29 | 3,5-di-tert-butyl-4-methoxy-N-(1-(morpholine-4- | A | 481.64 |
| | carbonyl)cyclohexyl)benzamide | | [M + Na] |
| 30 | 3,5-di-tert-butyl-4-methoxy-N-(1-(pyrrolidine-1- | A | 443.0 |
| | carbonyl)cyclohexyl)benzamide | | |
| 31 | 3,5-di-tert-butyl-4-methoxy-N-(1-(4- | A | 509.6 |
| | methoxybenzylcarbamoyl)cyclohexyl)benzamide | | |
| 32 | N-(1-(benzylcarbamoyl)cyclohexyl)-3,5-di-tert-butyl-4- | A | 479.6 |
| | methoxybenzamide | | |
| 33 | 3,5-di-tert-butyl-4-methoxy-N-(1-(3- | A | 547.7 |
| | (trifluoromethyl)benzylcarbamoyl)cyclohexyl)benzamide | | |
| 34 | 1-(3,5-di-tert-butylbenzamido)cyclohexanecarboxylic acid | A(SYNTHETIC | 360.3 |
| | | INTERMEDIATE) | |
| 35 | 3,5-di-tert-butyl-N-(1-(methylcarbamoyl)cyclohexyl)benzamide | E | 373.1 |
| 36 | N-(1-(benzylcarbamoyl)cyclopentyl)-3,5-di-tert-butylbenzamide | A | 435.6** |
| 37 | 3,5-di-tert-butyl-N-(1-(4- | E | 465.6** |
| | methoxybenzylcarbamoyl)cyclopentyl)benzamide | | |
| 38 | 3,5-di-tert-butyl-4-methoxy-N-(1-(4- | E | 495.6** |
| | methoxybenzylcarbamoyl)cyclopentyl)benzamide | | |
| 39 | 3,5-di-tert-butyl-N-(1-(3- | E | 503.6** |
| | (trifluoromethyl)benzylcarbamoyl)cyclopentyl)benzamide | | |
| 40 | 3,5-bis(2-cyanopropan-2-yl)-N-(1-(3- | A | 539.4 |
| | (trifluoromethyl)benzylcarbamoyl)cyclohexyl)benzamide | | |
| 41 | N-(1-(benzylcarbamoyl)cyclohexyl)-3,5-bis(2-cyanopropan-2- | A | 471.7 |
| | yl)benzamide | | |
| 42 | N-(3,5-di-tert-butylphenyl)-1-(2,4- | A | 457.4 |
| | difluorobenzylamino)cyclohexanecarboxamide | | |
| 43 | 1-(benzylamino)-N-(3,5-di-tert- | A | 421.5 |
| | butylphenyl)cyclohexanecarboxamide | | |
| 44 | N-(1-(benzylcarbamoyl)cyclohexyl)-3,5-di-tert-butylbenzamide | A | * |
| 45 | 3,5-di-tert-butyl-N-(1-(4- | A | * |
| | methoxybenzylcarbamoyl)cyclohexyl)benzamide | | |
| 46 | 3,5-di-tert-butyl-N-(1-(4- | A | * |
| | chlorobenzylcarbamoyl)cyclohexyl)benzamide | | |
| 47 | 3,5-di-tert-butyl-N-(1-(pyridin-2- | A | * |
| | ylmethylcarbamoyl)cyclohexyl)benzamide | | |
| 48 | 3,5-di-tert-butyl-N-(1-(4- | A | * |
| | (dimethylamino)benzylcarbamoyl)cyclohexyl)benzamide | | |
| 49 | 3,5-di-tert-butyl-N-(1-(4- | A | * |
| | fluorobenzylcarbamoyl)cyclohexyl)benzamide | | |
| 50 | 3,5-di-tert-butyl-N-(1-(2,4- | A | * |
| | difluorobenzylcarbamoyl)cyclohexyl)benzamide | | |
| 51 | 3,5-di-tert-butyl-N-(1-((5-methylisoxazol-3- | A | * |
| | yl)methylcarbamoyl)cyclohexyl)benzamide | | |
| 52 | N-(2-(benzylcarbamoyl)-2,3-dihydro-1H-inden-2-yl)-2,6-di-tert- | E | 484.6** |
| | butylisonicotinamide | | |
| 53 | 2-(3,5-di-tert-butylbenzamido)-N-(3-(trifluoromethyl)benzyl)-2,3- | E | 551.5** |
| | dihydro-1H-indene-2-carboxamide | | |
| 54 | 2,6-di-tert-butyl-N-(2-(3-(trifluoromethyl)benzylcarbamoyl)-2,3- | E | 552.5** |
| | dihydro-1H-inden-2-yl)isonicotinamide | | |
| 55 | 2-(4-(2-cyanopropan-2-yl)benzamido)-N-(3- | E | 506.5** |
| | (trifluoromethyl)benzyl)-2,3-dihydro-1H-indene-2-carboxamide | | |
| 56 | N-(1-(benzylcarbamoyl)cyclohexyl)-2,6-di-tert- | E | * |
| | butylisonicotinamide | | |
| 57 | N-benzyl-1-(3,5-di-tert- | G | * |
| | butylbenzylamino)cyclohexanecarboxamide | | |
| 58 | 1-(tert-butoxycarbonyl)-4-(2,6-di-tert- | A | 462.4 |
| | butylisonicotinamido)piperidine-4-carboxylic acid | | |
| 59 | tert-butyl 4-(benzylcarbamoyl)-4-(2,6-di-tert- | A | 551.7 |
| | butylisonicotinamido)piperidine-1-carboxylate | | |
| 60 | 1-(3,5-di-tert-butylbenzylamino)-N-(3- | G | * |
| | (trifluoromethyl)benzyl)cyclohexanecarboxamide | | |
| 61 | 3,5-bis(cyanomethyl)-N-(1-(3- | E | * |
| | (trifluoromethyl)benzylcarbamoyl)cyclohexyl)benzamide | | |
| 62 | 3,5-di-tert-butyl-N-(1-(3- | H | 517.4 |
| | (trifluoromethyl)benzylcarbamoyl)cyclohexyl)benzamide | | |
| 63 | 2,2′-(5-(1-(benzylcarbamoyl)cyclohexylcarbamoyl)-1,3- | E | 619.8 |
| | phenylene)bis(N-tert-butyl-2-methylpropanamide) | | |
| 64 | N-(4-(benzylcarbamoyl)piperidin-4-yl)-2,6-di-tert- | H(SYNTHETIC | 451.5 |
| | butylisonicotinamide | INTERMEDIATE) | |
| 65 | N-(1-(benzylcarbamoyl)cyclohexyl)-3,5-di-tert-butyl-4- | A | 465.5 |
| | hydroxybenzamide | | |
| 66 | 2,6-di-tert-butyl-N-(1-(pyrrolidine-1- | A | 414.7 |
| | carbonyl)cyclohexyl)isonicotinamide | | |
| 67 | 3,5-di-tert-butyl-4-hydroxy-N-(1-(4- | A | 495.5 |
| | methoxybenzylcarbamoyl)cyclohexyl)benzamide | | |
| 68 | 3,5-di-tert-butyl-N-(1-((1,5-dimethyl-1H-pyrazol-3- | A | 467.6** |
| | yl)methylcarbamoyl)cyclohexyl)benzamide | | |
| 69 | N-(1-(4-benzylpiperazine-1-carbonyl)cyclohexyl)-2,6-di-tert- | A | 519.7** |
| | butylisonicotinamide | | |
| 70 | N-(1-(benzylcarbamoyl)cyclohexyl)-3-tert-butyl-5-(2- | E | * |
| | cyanopropan-2-yl)benzamide | | |
| 71 | 1-(3,5-di-tert-butyl-4-methoxybenzylamino)-N-(3- | G | * |
| | (trifluoromethyl)benzyl)cyclohexanecarboxamide | | |
| 72 | N-benzyl-1-(3,5-di-tert-butyl-4- | G | * |
| | methoxybenzylamino)cyclohexanecarboxamide | | |
| 73 | N-(1-(3,5-di-tert-butyl-4-methoxybenzylcarbamoyl)cyclohexyl)-3- | G | * |
| | (trifluoromethyl)benzamide | | |
| 74 | N-(1-(3,5-di-tert-butyl-4-methoxybenzylcarbamoyl)cyclohexyl)-4- | E | * |
| | methoxybenzamide | | |
| 75 | 3,5-di-tert-butyl-N-(1-(pyridin-4- | H | * |
| | ylcarbamoyl)cyclohexyl)benzamide | | |
| 76 | 2,2′-(5-(1-(3- | E | * |
| | (trifluoromethyl)benzylcarbamoyl)cyclohexylcarbamoyl)-1,3- | | |
| | phenylene)bis(N-tert-butyl-2-methylpropanamide) | | |
| 77 | 2,6-di-tert-butyl-N-(1-(4- | A | 493.7** |
| | (dimethylamino)benzylcarbamoyl)cyclohexyl)isonicotinamide | | |
| 78 | 2,6-di-tert-butyl-N-(1-((1,5-dimethyl-1H-pyrazol-3- | A | 468.6** |
| | yl)methylcarbamoyl)cyclohexyl)isonicotinamide | | |
| 79 | 2,6-di-tert-butyl-N-(1-((5-methylisoxazol-3- | A | 455.6** |
| | yl)methylcarbamoyl)cyclohexyl)isonicotinamide | | |
| 80 | 2,6-di-tert-butyl-N-(1-(thiazol-2- | A | 457.6** |
| | ylmethylcarbamoyl)cyclohexyl)isonicotinamide | | |
| 81 | 2,6-di-tert-butyl-N-(1-((5-methylpyrazin-2- | A | 466.6** |
| | yl)methylcarbamoyl)cyclohexyl)isonicotinamide | | |
| 82 | 3,5-di-tert-butyl-4-methoxy-N-(1-(4-phenylpiperazine-1- | A | 534.7** |
| | carbonyl)cyclohexyl)benzamide | | |
| 83 | N-(1-(4-benzylpiperazine-1-carbonyl)cyclohexyl)-3,5-di-tert-butyl- | A | 548.7** |
| | 4-methoxybenzamide | | |
| 84 | 3,5-di-tert-butyl-N-(1-(4- | A | 522.7** |
| | (dimethylamino)benzylcarbamoyl)cyclohexyl)-4- | | |
| | methoxybenzamide | | |
| 85 | 3,5-di-tert-butyl-N-(1-((1,5-dimethyl-1H-pyrazol-3- | A | 497.7** |
| | yl)methylcarbamoyl)cyclohexyl)-4-methoxybenzamide | | |
| 86 | 3,5-di-tert-butyl-4-methoxy-N-(1-((5-methylisoxazol-3- | A | 484.6** |
| | yl)methylcarbamoyl)cyclohexyl)benzamide | | |
| 87 | 3,5-di-tert-butyl-4-methoxy-N-(1-(thiazol-2- | A | 486.6** |
| | ylmethylcarbamoyl)cyclohexyl)benzamide | | |
| 88 | 3,5-di-tert-butyl-4-methoxy-N-(1-((5-methylpyrazin-2- | A | 495.6** |
| | yl)methylcarbamoyl)cyclohexyl)benzamide | | |
| 89 | 2,6-di-tert-butyl-N-(1-(4- | A | 467.6** |
| | methoxybenzylcarbamoyl)cyclopentyl)isonicotinamide | | |
| 90 | 1-(2,6-di-tert-butylisonicotinimidamido)-N-((5-methylisoxazol-3- | B | 441.6** |
| | yl)methyl)cyclopentanecarboxamide | | |
| 91 | 2,6-di-tert-butyl-N-(1-((5-methylpyrazin-2- | A | 452.6** |
| | yl)methylcarbamoyl)cyclopentyl)isonicotinamide | | |
| 92 | 2,6-di-tert-butyl-N-(1-(pyrrolidine-1- | A | 400.6** |
| | carbonyl)cyclopentyl)isonicotinamide | | |
| 93 | 2,6-di-tert-butyl-N-(1-(4-methylpiperazine-1- | A | 429.6** |
| | carbonyl)cyclopentyl)isonicotinamide | | |
| 94 | 2,6-di-tert-butyl-N-(1-(morpholine-4- | A | 416.6** |
| | carbonyl)cyclopentyl)isonicotinamide | | |
| 95 | N-(1-(benzylcarbamoyl)cyclopentyl)-2,6-di-tert- | A | 436.6** |
| | butylisonicotinamide | | |
| 96 | 3,5-di-tert-butyl-4-methoxy-N-(1- | G | 465.2 |
| | (phenylcarbamoyl)cyclohexyl)benzamide | | |
| 97 | N-(4-(benzylcarbamoyl)-1-methylpiperidin-4-yl)-2,6-di-tert- | I | 465.4 |
| | butylisonicotinamide | | |
| 98 | 2,6-di-tert-butyl-N-(1-(morpholine-4- | A | 430.6** |
| | carbonyl)cyclohexyl)isonicotinamide | | |
| 99 | 2,6-di-tert-butyl-N-(1-(3- | A | 487.7** |
| | morpholinopropylcarbamoyl)cyclohexyl)isonicotinamide | | |
| 100 | 2,6-di-tert-butyl-N-(1-(pyridin-4- | A | 451.6** |
| | ylmethylcarbamoyl)cyclohexyl)isonicotinamide | | |
| 101 | 2,6-di-tert-butyl-N-(1- | A | 374.6** |
| | (methylcarbamoyl)cyclohexyl)isonicotinamide | | |
| 102 | 3,5-di-tert-butyl-N-(1-(3- | A | 486.7** |
| | morpholinopropylcarbamoyl)cyclohexyl)benzamide | | |
| 103 | 3,5-di-tert-butyl-N-(1-(pyridin-3- | A | 450.6** |
| | ylmethylcarbamoyl)cyclohexyl)benzamide | | |
| 104 | 3,5-di-tert-butyl-4-methoxy-N-(1-(4-methylpiperazine-1- | A | 472.7** |
| | carbonyl)cyclohexyl)benzamide | | |
| 105 | 3,5-di-tert-butyl-4-methoxy-N-(1-(3- | A | 516.8** |
| | morpholinopropylcarbamoyl)cyclohexyl)benzamide | | |
| 106 | 2,6-di-tert-butyl-N-(1-(4- | A | 528.5** |
| | (methylsulfonyl)benzylcarbamoyl)cyclohexyl)isonicotinamide | | |
| 107 | 3,5-di-tert-butyl-N-(1-(4- | A | 527.6** |
| | (methylsulfonyl)benzylcarbamoyl)cyclohexyl)benzamide | | |
| 108 | 3,5-di-tert-butyl-4-methoxy-N-(1-(4- | A | 557.6** |
| | (methylsulfonyl)benzylcarbamoyl)cyclohexyl)benzamide | | |
| 109 | 3,5-di-tert-butyl-4-hydroxy-N-(1-(4- | E | 453.5 |
| | methoxybenzylcarbamoyl)cyclopropyl)benzamide | | |
| 110 | 3,5-di-tert-butyl-4-methoxy-N-(1-(4- | E | 467.6 |
| | methoxybenzylcarbamoyl)cyclopropyl)benzamide | | |
| 111 | 3,5-di-tert-butyl-4-hydroxy-N-(1- | G | 451.6 |
| | (phenylcarbamoyl)cyclohexyl)benzamide | | |
| 112 | 3,5-di-tert-butyl-4-hydroxy-N-(1-(4- | G | 543.8 |
| | (methylsulfonyl)benzylcarbamoyl)cyclohexyl)benzamide | | |
| 113 | 2,6-di-tert-butyl-N-(4-(4-methoxybenzylcarbamoyl)tetrahydro-2H- | E | 482.5 |
| | pyran-4-yl)isonicotinamide | | |
| 114 | 4-(3,5-di-tert-butyl-4-methoxybenzamido)-N-(4- | E | 511.6 |
| | methoxybenzyl)tetrahydro-2H-pyran-4-carboxamide | | |
| 115 | 4-(3,5-bis(2-cyanopropan-2-yl)benzamido)-N-(4- | E | 503.6 |
| | methoxybenzyl)tetrahydro-2H-pyran-4-carboxamide | | |
| 116 | 3,5-di-tert-butyl-4-hydroxy-N-(1-((5-methylisoxazol-3- | A | 428.3 |
| | yl)methylcarbamoyl)cyclopropyl)benzamide | | |
| 117 | 3,5-di-tert-butyl-4-hydroxy-N-(1-(4- | A | 516.4 |
| | (methylsulfonamido)benzylcarbamoyl)cyclopropyl)benzamide | | |
| 118 | 3-(2-cyanopropan-2-yl)-4-methoxy-N-(1-(3- | E | 502.4 |
| | (trifluoromethyl)benzylcarbamoyl)cyclohexyl)benzamide | | |
| 119 | 2-(2-cyanopropan-2-yl)-N-(1-(3- | E | 473.7 |
| | (trifluoromethyl)benzylcarbamoyl)cyclohexyl)isonicotinamide | | |
| 120 | 2-tert-butyl-N-(4-(3-(trifluoromethyl)phenylcarbamoyl)tetrahydro- | E | 450.7 |
| | 2H-pyran-4-yl)isonicotinamide | | |
| 121 | 3-(1-amino-2-methyl-1-oxopropan-2-yl)-N-(1-(3- | E | * |
| | (trifluoromethyl)benzylcarbamoyl)cyclohexyl)benzamide | | |
| 122 | N-(1-(benzylcarbamoyl)cyclohexyl)-4-(2-cyanopropan-2- | E | * |
| | yl)benzamide | | |
| 123 | N-(1-(benzylcarbamoyl)cyclohexyl)-3-(2-cyanopropan-2- | E | * |
| | yl)benzamide | | |
| 124 | 3-(2-cyanopropan-2-yl)-N-(1-(3- | E | * |
| | (trifluoromethyl)benzylcarbamoyl)cyclohexyl)benzamide | | |
| 125 | N-(1-(benzylcarbamoyl)cyclohexyl)-4-(1-(tert-butylamino)-2- | E | * |
| | methyl-1-oxopropan-2-yl)benzamide | | |
| 126 | 4-(1-(tert-butylamino)-2-methyl-1-oxopropan-2-yl)-N-(1-(3- | E | * |
| | (trifluoromethyl)benzylcarbamoyl)cyclohexyl)benzamide | | |

* Note: Where [M + H] is not available, the compound was isolated by Mass Directed RPLC.
**Note: These ones are as above, but the Crude [M + H] was recorded and is the mass in the cell.

### Example 4

### Assay Example 1: Fluorescent Assay for Cav2.2 Channels Using Potassium Depolarization to Initiate Channel Opening

Human Cav2.2 channels were stably expressed in HEK293 cells along with alpha2-delta and beta subunits of voltage-gated calcium channels. An inwardly rectifying potassium channel (Kir2.3) was also expressed in these cells to allow more precise control of the cell membrane potential by extracellular potassium concentration. At low bath potassium concentration, the membrane potential is relatively negative, and is depolarized as the bath potassium concentration is raised. In this way, the bath potassium concentration can be used to regulate the voltage-dependent conformations of the channels. Compounds are incubated with cells in the presence of low (4 mM) potassium or elevated (12, 25 or 30 mM) potassium to determine the affinity for compound block of resting (closed) channels at 4 mM potassium or affinity for block of open and inactivated channels at 12, 25 or 30 mM potassium. After the incubation period, Cav2.2 channel opening is triggered by addition of higher concentration of potassium (70 mM final concentration) to further depolarize the cell. The degree of state-dependent block can be estimated from the inhibitory potency of compounds after incubation in different potassium concentrations.

Calcium influx through Cav2.2 channels is determined using a calcium sensitive fluorescent dye in combination with a fluorescent plate reader. Fluorescent changes were measured with either a VIPR (Aurora Instruments) or FLIPR (Molecular Devices) plate reader.

### Protocol

1. Seed cells in Poly-D-Lysine Coated 96 or 384-well plate and keep in a 37° C.-10% CO₂ incubator overnight
2. Remove media, wash cells with 0.2 mL (96-well plate) or 0.05 mL (384-well plate) Dulbecco's Phosphate Buffered Saline (D-PBS) with calcium & magnesium (Invitrogen; 14040)
3. Add 0.1 mL (96-well plate) or 0.05 mL (384-well plate) of 4 μM fluor-4 (Molecular Probes; F-14202) and 0.02% Pluronic acid (Molecular Probes; P-3000) prepared in D-PBS with calcium & magnesium (Invitrogen; 14040) supplemented with 10 mM Glucose & 10 mM Hepes/NaOH; pH 7.4
4. Incubate in the dark at 25° C. for 60-70 min
5. Remove dye, wash cells with 0.1 mL (96-well plate) or 0.06 mL (384-well plate) of 4, 12, 25, or 30 mM Potassium Pre-polarization Buffer (PPB)
6. Add 0.1 mL (96-well plate) or 0.03 mL (384-well plate) of 4, 12, 25, 30 mM PPB with or without test compound
7. Incubate in the dark at 25° C. for 30 min
8. Read cell plate on VIPR instrument, Excitation=480 nm, Emission=535 nm
9. With VIPR continuously reading, add 0.1 mL (96-well plate) or 0.03 mL (384-well plate) of Depolarization Buffer (DB), which is 2× the final assay concentration, to the cell plate.

| | | | | |
|---|---|---|---|---|
| 4 mM | 12 mM | 25 mM PPB | 30 mM PPB | 140 mM K DB |
| PPB | PPB | | | |
| 146 mM | 138 mM | 125 mM NaCl | 120 mM NaCl | 10 mM NaCl |
| NaCl | NaCl | | | |
| 4 mM KCl | 12 mM | 25 mM KCl | 30 mM KCl | 140 mM KCl |
| | KCl | | | |
| 0.8 mM | 0.8 mM | 0.8 mM CaCl₂ | 0.8 mM CaCl₂ | 0.8 mM CaCl₂ |
| CaCl₂ | CaCl₂ | | | |
| 1.7 MgCl₂ | 1.7 MgCl₂ | 1.7 MgCl₂ | 1.7 MgCl₂ | 1.7 MgCl₂ |
| 10 HEPES | 10 HEPES | 10 HEPES | 10 HEPES | 10 HEPES |
| pH = 7.2 | pH = 7.2 | pH = 7.2 | pH = 7.2 | pH = 7.2 |

### Example 5

### Assay Example 2: Electrophysiological Measurement of Block of Cav2.2 Channels Using Automated Electrophysiology Instruments

Block of N-type calcium channels is evaluated utilizing the IonWorks HT 384 well automated patch clamp electrophysiology device. This instrument allows synchronous recording from 384 well (48 at a time). A single whole cell recording is made in each well. Whole cell recording is established by perfusion of the internal compartment with amphotericin B.

The voltage protocol is designed to detect use-dependent block. A 2 Hz train of depolarizations (twenty 25 ms steps to +20 mV). The experimental sequence consists of a control train (pre-compound), incubation of cells with compound for 5 minutes, followed by a second train (post-compound). Use dependent block by compounds is estimated by comparing fractional block of the first pulse in the train to block of the 20^{th} pulse.

### Protocol:

Parallel patch clamp electrophysiology is performed using IonWorks HT (Molecular Devices Corp) essentially as described by Kiss and colleagues (Kiss et al. 2003; Assay and Drug Development Technologies, 1:127-135). Briefly, a stable HEK 293 cell line (referred to as CBK) expressing the N-type calcium channel subunits α_{1B}, α₂δ, β₃ₐ) and an inwardly rectifying potassium channel (Kᵢᵣ2.3) is used to record barium current through the N-type calcium channel. Cells are grown in T75 culture plates to 60-90% confluence before use. Cells are rinsed 3× with 10 mL PBS (Ca/Mg-free) followed by addition of 1.0 mL 1× trypsin to the flask. Cells are incubated at 37° C. until rounded and free from plate (usually 1-3 min). Cells are then transferred to a 15 mL conical tube with 13 ml of CBK media containing serum and antibiotics and spun at setting 2 on a table top centrifuge for 2 min. The supernatant is poured off and the pellet of cells is resuspended in external solution (in mM): 120 NaCl, 20 BaCl₂, 4.5 KCl, 0.5 MgCl₂, 10 HEPES, 10 Glucose, pH 7.4). The concentration of cells in suspension is adjusted to achieve 1000-3000 cells per well. Cells are used immediately once they have been resuspended. The internal solution is (in mM): 100 K-Gluconate, 40 KCl, 3.2 MgCl₂, 3 EGTA, 5 HEPES, pH 7.3 with KOH. Perforated patch whole cell recording is achieved by adding the perforating agent amphotericin B to the internal solution. A 36 mg/mL stock of amphtericin B is made fresh in DMSO for each run. 166 μL of this stock is added to 50 mL of internal solution yielding a final working solution of 120 μg/mL.

Voltage protocols and the recording of membrane currents are performed using the IonWorks HT software/hardware system. Currents are sampled at 1.25 kHz and leakage subtraction is performed using a 10 mV step from the holding potential and assuming a linear leak conductance. No correction for liquid junction potentials is employed. Cells are voltage clamped at −70 mV for 10 s followed by a 20 pulse train of 25 ms steps to +20 mV at 2 Hz. After a control train, the cells are incubated with compound for 5 minutes and a second train is applied. Use dependent block by compounds is estimated by comparing fractional block of the first pulse to block of the 20^{th} pulse. Wells with seal resistances less than 70 MOhms or less than 0.1 nA of Ba current at the test potential (+20 mV) are exluded from analysis. Current amplitudes are calculated with the IonWorks software. Relative current, percent inhibition and IC₅₀s are calculated with a custom Excel/Sigmaplot macro.

Compounds are added to cells with a fluidics head from a 96-well compound plate. To compensate for the dilution of compound during addition, the compound plate concentration is 3× higher than the final concentration on the patch plate.

Two types of experiments are generally performed: screens and titrations. In the screening mode, 10-20 compounds are evaluated at a single concentration (usually 3 μM). The percent inhibition is calculated from the ratio of the current amplitude in the presence and absence of compound, normalized to the ratio in vehicle control wells. For generation of IC₅₀s, a 10-point titration is performed on 2-4 compounds per patch plate. The range of concentrations tested is generally 0.001 to 20 μM. IC₅₀s are calculated from the fits of the Hill equation to the data. The form of the Hill equation used is: Relative Current=(Max−Min)/((1+(conc/IC50)̂slope)+Min). Vehicle controls (DMSO) and 0.3 mM CdCl₂ (which inhibits the channel completely) are run on each plate for normalization purposes and to define the Max and Min.

### Example 6

### Assay Example 3: Electrophysiological Measurement of Block of Cav2.2 Channel Using Whole Cell Voltage Clamp and Using PatchXpress Automated Electrophysiology Instrument

Block of N-type calcium channels is evaluated utilizing manual and automated (PatchXpress) patch clam electrophysiology. Voltage protocols are designed to detect state-dependent block. Pulses (50 ms) are applied at a slow frequency (0.067 Hz) from polarized (−90 mV) or depolarized (−40 mV) holding potentials. Compounds which preferentially block inactivated/open channels over resting channels will have higher potency at −40 mV compared to −90 mV.

### Protocol:

A stable HEK 293 cell line (referred to as CBK) expressing the N-type calcium channel subunits (α_{1B}, α₂δ, β₃ₐ) and an inwardly rectifying potassium channel (Kᵢᵣ2.3) is used to record barium current through the N-type calcium channel. Cells are grown either on poly-D-lysine coated coverglass (manual EP) or in T75 culture plates (PatchXpress). For the PatchExpress, cells are released from the flask using trypsin. In both cases, the external solution is (in mM): 130 CsCl₂, 10 EGTA, 10 HEPES, 2 MgCl₂, 3 MgATP, pH 7.3 with CsOH.

Barium currents are measured by manual whole-cell patch clamp using standard techniques (Hamill et. Al. Pfluegers Archiv 391:85-100 (1981)). Microelectrodes are fabricated from borosilicate glass and fire-polished. Electrode resistances are generally 2 to 4 MOhm when filled with the standard internal saline. The reference electrode is a silver-silver chloride pellet. Voltages are not corrected for the liquid junction potential between the internal and external solutions and leak is subtracted using the P/n procedure. Solutions are applied to cells by bath perfusion via gravity. The experimental chamber volume is ˜0.2 mL and the perfusion rate is 0.5-2 mL/min. Flow of suction through the chamber is maintained at all times. Measurement of current amplitudes is performe with PULSEFIT software (HEKA Elektronik).

PatchXpress (Molecular Devices is a 16-well whole-cell automated patch clamp device that operates asynchronously with fully integrated fluidics. High resistance (gigaohm) seals are achieved with 50-80% success. Capacitance and series resistance compensation is automated. No correction for liquid junction potentials is employed. Leak is subtracted using the P/n procedure. Compounds are added to cells with a pipettor from a 96-well compound plate. Voltage protocols and the recording of membrane currents are performed using the PatchXpress software/hardware system. Current amplitudes are calculated with DataXpress software.

In both manual and automated patch clamp, cells are voltage clamped at −4 mV or −90 mV and 50 ms pulses to +20 mV are applied every 15 sec (0.067 Hz). Compounds are added in escalating doses to measure % inhibition. Percent inhibition is calculated from the ratio of the current amplitude in the presence and absence of compound. When multiple doses are achieved per cell, IC₅₀s are calculated. The range of concentrations tested is generally 0.1 to 30 μM. IC₅₀s are calculated from the fits of the Hill equation to the data. The form of the Hill equation used is: Relative Current=1/(1+(conc/IC₅₀)̂slope).

### Example 7

### Assay Example 4: Assay for Cav3.1 and Cav3.2 Channels

The T-type calcium channel blocking activity of the compounds of this invention may be readily determined using the methodology well known in the art described by Xia, et al., Assay and Drug Development Tech., 1(5), 637-645 (2003).

In a typical experiment ion channel function from HEK 293 cells expressing the T-type channel alpha-1G, H, or I (CaV 3.1, 3.2, 3.3) is recorded to determine the activity of compounds in blocking the calcium current mediated by the T-type channel alpha-1G, H, or I (CaV 3.1, 3.2, 3.3). In this T-type calcium (Ca²⁺) antagonist voltage-clamp assay calcium currents are elicited from the resting state of the human alpha-1G, H, or I (CaV 3.1, 3.2, 3.3) calcium channel as follows. Sequence information for T-type (Low-voltage activated) calcium channels are fully disclosed in e.g., U.S. Pat. No. 5,618,720, U.S. Pat. No. 5,686,241, U.S. Pat. No. 5,710,250,U.S. Pat. No. 5,726,035, U.S. Pat. No. 5,792,846, U.S. Pat. No. 5,846,757, U.S. Pat. No. 5,851,824, U.S. Pat. No. 5,874,236, U.S. Pat. No. 5,876,958, U.S. Pat. No. 6,013,474, U.S. Pat. No. 6,057,114, U.S. Pat. No. 6,096,514, WO 99/28342, and J. Neuroscience, 19(6):1912-1921 (1999). Cells expressing the t-type channels were grown in H3D5 growth media which is comprised DMEM, 6% bovine calf serum (HYCLONE), 30 micromolar Verapamil, 200 microgram/ml Hygromycin B, 1× Penicillin/Streptomycin. Glass pipettes are pulled to a tip diameter of 1-2 micrometer on a pipette puller. The pipettes are filled with the intracellular solution and a chloridized silver wire is inserted along its length, which is then connected to the headstage of the voltage-clamp amplifier. Trypsinization buffer was 0.05% Trypsin, 0.53 mM EDTA. The extracellular recording solution consists of (mM): 130 mM NaCl, 4 mM KCl, 1 mM MgCl₂, 2 mM CaCl₂, 10 mM HEPES, 30 Glucose, pH 7.4. The internal solution consists of (mM): 135 mM CsMeSO₄, 1 MgCl₂, 10 CsCl, 5 EGTA, 10 HEPES, pH 7.4, or 135 mM CsCl, 2 MgCl₂, 3 MgATP, 2 Na₂ATP, 1 Na₂GTP, 5 EGTA, 10 HEPES, pH 7.4. Upon insertion of the pipette tip into the bath, the series resistance is noted (acceptable range is between 1-4 megaohm). The junction potential between the pipette and bath solutions is zeroed on the amplifier. The cell is then patched, the patch broken, and, after compensation for series resistance (>=80%), the voltage protocol is applied while recording the whole cell Ca²⁺ current response. Voltage protocols: (1) −80 mV holding potential every 20 seconds pulse to −20 mV for 40 msec duration; the effectiveness of the drug in inhibiting the current mediated by the channel is measured directly from measuring the reduction in peak current amplitude initiated by the voltage shift from −80 mV to −20 mV; (2). −100 mV holding potential every 15 seconds pulse to −20 mV for 40 msec duration; the effectiveness of the drug in inhibiting the current mediated by the channel is measured directly from measuring the reduction in peak current amplitude initiated by the shift in potential from −100 mV to −30 mV. The difference in block at the two holding potentials was used to determine the effect of drug at differing levels of inactivation induced by the level of resting state potential of the cells. After obtaining control baseline calcium currents, extracellular solutions containing increasing concentrations of a test compound are washed on. Once steady state inhibition at a given compound concentration is reached, a higher concentration of compound is applied. % inhibition of the peak inward control Ca²⁺ current during the depolarizing step to −20 mV is plotted as a function of compound concentration.

### Example 8

### In vitro Activity

The intrinsic T-type calcium channel antagonist activity of a compound which may be used in the present invention may be determined by these assays.

TABLE 2
In Vitro Activity

| | Cav 2.2 Activity | | |
|---|---|---|---|
| | % Inh at | % Inh at | IC₅₀ at 30 |
| ID | 10 μM | 30 μM | mM K (μM) |
| 1 | 95.15 | 104.32 | 1.211 |
| 2 | 99.05 | 103.55 | 1.41 |
| 3 | 89.75 | 98.79 | 1.619 |
| 4 | 68.04 | 76.05 | 0.3552 |
| 5 | 94.6 | 96.41 | 0.1272 |
| 6 | 54.72 | 65.98 | |
| 7 | 99.32 | 102.24 | 0.1279 |
| 8 | 100.04 | 98.95 | 0.1293 |
| 9 | 45.68 | 53.36 | |
| 10 | 95.69 | 101.43 | 0.1839 |
| 11 | 91.94 | 92.23 | 0.336 |
| 12 | 98.61 | 101.35 | 0.05502 |
| 13 | 96.87 | 99.84 | 0.03411 |
| 14 | 80.74 | 81.87 | |
| 15 | 48.24 | 65.93 | |
| 16 | 89.27 | 93.02 | |
| 17 | 48.17 | 90.62 | |
| 18 | 40.68 | 34.52 | |
| 19 | 60.31 | | |
| 20 | 100.23 | | 0.1022 |
| 21 | 97.1 | | 0.143 |
| 22 | 95.08 | | 0.4024 |
| 23 | 87.44 | | |
| 24 | 79.22 | | 0.7671 |
| 25 | 70.46 | | 0.08736 |
| 26 | 49.02 | | |
| 27 | 101.9 | | 1.044 |
| 28 | 82.56 | 89.4 | 0.06927 |
| 29 | 85.81 | 90.54 | 0.06764 |
| 30 | 91.68 | 93.43 | 0.02525 |
| 31 | 87.51 | 104.57 | 0.002603 |
| 32 | 88.85 | 98.03 | 0.005597 |
| 33 | 81.37 | 90.51 | 0.03762 |
| 34 | 68.25 | 82.26 | |
| 35 | 92.48 | 98.63 | 1.261 |
| 36 | 91.96 | 96.4 | 0.2513 |
| 37 | 92.99 | 95.84 | 0.1528 |
| 38 | 97.78 | 98.98 | 0.0359 |
| 39 | 79.59 | 77.88 | 0.5134 |
| 40 | 79.98 | 77.62 | 0.3665 |
| 41 | 75.96 | 73.3 | 0.9854 |
| 42 | 70.57 | 80.15 | |
| 43 | 76.25 | 82.5 | |
| 44 | 92.52 | 97.26 | 0.06038 |
| 45 | 95.73 | 99.54 | 0.02519 |
| 46 | 96.59 | 97.86 | 0.07795 |
| 47 | 93.67 | 95.27 | 0.03558 |
| 48 | 88.74 | 90.29 | 0.3706 |
| 49 | 103.35 | 91.58 | 0.04416 |
| 50 | 99.64 | 102.23 | 0.0803 |
| 51 | 105.88 | 105.53 | 0.06353 |
| 52 | 82.22 | 91.34 | 0.8932 |
| 53 | 56.3 | 74.34 | |
| 54 | 46.16 | 68.4 | |
| 55 | 89 | 100.24 | |
| 56 | 84.39 | 92.62 | 0.3205 |
| 57 | 99.85 | 100.76 | 0.8683 |
| 58 | 37.61 | 77.92 | |
| 59 | 50.25 | 62.46 | |
| 60 | 74.81 | 85.8 | |
| 61 | 18.66 | 56.37 | |
| 62 | 94.26 | 88.14 | 0.09907 |
| 63 | 66.42 | 82.02 | |
| 64 | 69.49 | 104.48 | |
| 65 | 103.66 | 106.55 | 0.1234 |
| 66 | | | 0.96 |
| 67 | 94.37 | 98.6 | 0.0206 |
| 68 | 98.36 | 94.02 | 0.06493 |
| 69 | 88.26 | 95.27 | 0.538 |
| 70 | 89.82 | 96.28 | 0.02743 |
| 71 | 77.32 | 81.08 | 0.4772 |
| 72 | 97.85 | 97.36 | 0.1451 |
| 73 | 95.22 | 97.71 | 0.1243 |
| 74 | 98.88 | 106.02 | 0.05576 |
| 75 | 93.59 | 100.54 | 0.5446 |
| 76 | 80.47 | 87.84 | |
| 77 | 48.66 | 66.91 | 0.5742 |
| 78 | 79.45 | 88.61 | 0.02449 |
| 79 | 97.84 | 101.18 | 0.04332 |
| 80 | 98.38 | 103.61 | 0.1059 |
| 81 | 87.76 | 93.34 | 0.05353 |
| 82 | 92.56 | 101.29 | 0.07091 |
| 83 | 97.15 | 100.49 | 0.1335 |
| 84 | 25.55 | 53.47 | |
| 85 | 87.74 | 93.19 | 0.02674 |
| 86 | 99.14 | 103.64 | 0.009574 |
| 87 | 102.09 | 104.47 | 0.006038 |
| 88 | 96.34 | 96.66 | 0.01425 |
| 89 | 90.83 | 99.96 | 0.0546 |
| 90 | 90.95 | 98.53 | 0.07724 |
| 91 | 95.3 | 96.07 | 0.1478 |
| 92 | 89.52 | 92.16 | 0.225 |
| 93 | 83.94 | 85.51 | 0.8559 |
| 94 | 96.62 | 87.2 | 0.3288 |
| 95 | 94.61 | 101.14 | 0.132 |
| 96 | 77.7 | 82.64 | 0.1613 |
| 97 | 76.01 | 90.62 | 1.072 |
| 98 | | | 0.2482 |
| 99 | | | 0.7005 |
| 100 | | | 0.1541 |
| 101 | | | 0.1054 |
| 102 | | | 0.2529 |
| 103 | | | 0.06534 |
| 104 | | | 1.022 |
| 105 | | | 0.7043 |
| 106 | | | 0.3172 |
| 107 | | | 0.1432 |
| 108 | | | 0.08163 |
| 109 | 29.76 | 39.97 | 0.08773 |
| 110 | 100.29 | 101.11 | 0.1215 |
| 111 | 71.83 | 105.71 | 0.5505 |
| 112 | 96.72 | 102.35 | 0.1379 |
| 113 | 79.03 | 92.99 | 0.05939 |
| 114 | 90.81 | 92.39 | 0.0324 |
| 115 | 53.5 | 73.36 | |
| 116 | 92.46 | 98.03 | 0.9431 |
| 117 | 74.2 | 100.58 | 1.094 |
| 118 | 75.42 | 77.64 | 0.3877 |
| 119 | 63.14 | | |
| 120 | 61.18 | | |
| 121 | 18.59 | 70.76 | |
| 122 | 45.24 | 80.47 | |
| 123 | 53.46 | 27.26 | |
| 124 | 84.24 | 74.03 | 0.8099 |
| 125 | 89.61 | 85.76 | 0.7392 |
| 126 | 102.04 | 103.62 | 0.2733 |

### Example 9

### In Vivo Assay 1: Rodent CFA Model

Male Sprague Dawley rates (300-400 g) are administered 200 μL CFA (complete Freund's Adjuvant) three days prior to the study. CFA is mycobacterium tuberculosis suspended in saline (1:1; Sigma) to form an emulsion that contains 0.5 mg mycobacterium/mL. The CFA is injected into the plantar area of the left hind paw.

Rats are fasted the night before the study only for oral administration of the compounds. On the morning of the test day using a Ugo Basile apparatus, 2 baseline samples can be taken 1 hour apart. The rat is wrapped in a towel. Its paw is placed over a ball bearing and under the pressure device. A foot pedal is depressed to apply constant linear pressure. Pressure is stopped when the rat withdraws its paw, vocalizes, or struggles. The right paw is then tested. Rats can then be dosed with compound and tested at predetermined time points.

Compounds are prepared in DMSO (15%)/PEG300 (60%)/Water (25%) and were dosed in a volume of 2 mL/kg.

Percent maximal possible effect (% MPE) can be calculated as: (post-treatment−pre-treatment)/(pre-injury threshold−pre-treatment)×100. The % responder is the number of rats that have an MPE 30% at any time following compound administration. The effect of treatment can be determined by one-way ANOVA Repeated Measures Friedman Test with a Dunn's post test.

### Example 10

### In Vivo Assay 2: Formalin-Induced Pain Model

The effects of intrathecally delivered compounds of the invention on the rat formalin model can also be measured. The compounds can be reconstituted to stock solutions of approximately 10 mg/mL in propylene glycol. Typically eight Holtzman male rats of 275-375 g size are randomly selected per test article.

The following study groups can be used, with test article, vehicle control (propylene glycol) and saline delivered intraperitoneally (IP):

TABLE 3
Formalin Model Dose Groups

| Test/Control Article | Dose | Route | Rats per group |
|---|---|---|---|
| Compound | 30 mg/kg | IP | 6 |
| Propylene glycol | N/A | IP | 4 |
| Saline | N/A | IP | 7 |

N/A = Not Applicable

Prior to initiation of drug delivery baseline behavioral and testing data can be taken. At selected times after infusion of the Test or Control Article these data can then be again collected.

On the morning of testing, a small metal band (0.5 g) is loosely placed around the right hind paw. The rat is placed in a cylindrical Plexiglas chamber for adaptation a minimum of 30 minutes. Test Article or Vehicle Control Article is administered 10 minutes prior to formalin injection (50 μL of 5% formalin) into the dorsal surface of the right hindpaw of the rat. The animal is then placed into the chamber of the automated formalin apparatus where movement of the formalin injected paw is monitored and the number of paw flinches tallied by minute over the next 60 minutes (Malmberg, A. B., et al., Anesthesiology (1993) 79:270 281).

Results can be presented as Maximum Possible Effect ±SEM, where saline control=100%.

### Example 11

### In Vivo Assay 3: Spinal Nerve Ligation Model of Neuropathic Pain

Spinal nerve ligation (SNL) injury can be induced using the procedure of Kim and Chung, (Kim, S. H., et al., Pain (1992) 50:355-363) in male Sprague-Dawley rats (Harlan; Indianapolis, Ind.) weighing 200 to 300 grams. Anesthesia is induced with 2% halothane in O₂ at 2 L/min and maintained with 0.5% halothane in O₂. After surgical preparation of the rats and exposure of the dorsal vertebral column from L4 to S2, the L5 and L6 spinal nerves are tightly ligated distal to the dorsal root ganglion using 4-0 silk suture. The incision is closed, and the animals are allowed to recover for 5 days. Rats that exhibit motor deficiency (such as paw-dragging) or failure to exhibit subsequent tactile allodynia are excluded from further testing. Sham control rats undergo the same operation and handling as the experimental animals, but without SNL.

The assessment of tactile allodynia consists of measuring the withdrawal threshold of the paw ipsilateral to the site of nerve injury in response to probing with a series of calibrated von Frey filaments. Each filament is applied perpendicularly to the plantar surface of the ligated paw of rats kept in suspended wire-mesh cages. Measurements are taken before and after administration of drug or vehicle. Withdrawal threshold is determined by sequentially increasing and decreasing the stimulus strength (“up and down” method), analyzed using a Dixon non-parametric test (Chaplan S. R., et al., J Pharmacol Exp Ther (1994) 269:1117-1123), and expressed as the mean withdrawal threshold.

The method of Hargreaves and colleagues (Hargreaves, K., et al., Pain (1988) 32:77-8) can be employed to assess paw-withdrawal latency to a thermal nociceptive stimulus. Rats are allowed to acclimate within a plexiglass enclosure on a clear glass plate maintained at 30° C. A radiant heat source (i.e., high intensity projector lamp) is then activated with a timer and focused onto the plantar surface of the affected paw of nerve-injured or carrageenan-injected rats. Paw-withdrawal latency can be determined by a photocell that halts both lamp and timer when the paw is withdrawn. The latency to withdrawal of the paw from the radiant heat source is determined prior to carrageenan or L5/L5 SNL, 3 hours after carrageenan or 7-21 days after L5/L6 SNL but before drug and after drug administration. A maximal cut-off of 40 seconds is employed to prevent tissue damage. Paw withdrawal latencies can be thus determined to the nearest 0.1 second. Reversal of thermal hyperalgesia is indicated by a return of the paw withdrawal latencies to the pre-treatment baseline latencies (i.e., 21 seconds). Anti-nociception is indicated by a significant (p<0.05) increase in paw withdrawal latency above this baseline. Data is converted to % anti hyperalgesia or % anti-nociception by the formula: (100×(test latency−baseline latency)/(cut-off−baseline latency) where cut-off is 21 seconds for determining anti-hyperalgesia and 40 seconds for determining anti-nociception.

1. A compound of formula 1: 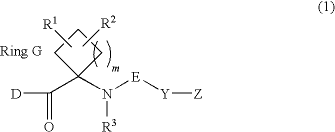
or a pharmaceutically acceptable salt or conjugate thereof, wherein
m is 0-3;
Ring G optionally contains O, S or NR as a ring member in place of one carbon atom,
R¹ and R² are independently H or selected from a group consisting of optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl group, halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, and NO₂,
or R¹ and R² are joined together to form an optionally substituted 5-6 membered ring fused to ring G; wherein the 5-6 membered ring fused to ring G may contain one or more N, O or S as a ring member; and
R³ is H or C1-C4 alkyl, C2-C4 alkenyl, or C2-C4 alkynyl, each of which is optionally substituted with one or more ═O, halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, and NO₂;
E is —C(═O)— or optionally substituted C1-C4 alkylene;
D is OH or D is NR⁴R⁵, wherein R⁴ is H and R⁵ is optionally substituted C1-C4 alkyl or -L-Q, wherein
L is a bond or optionally substituted C1-C4 alkylene;
Q is an optionally substituted 5-6 membered ring, which may contain up to 4 heteroatoms as ring members, each independently selected from the group consisting of O, S, N and NR⁶,
or R⁴ and R⁵ are joined to form an optionally substituted 5-6 membered saturated ring, which may contain up to 2 heteroatoms selected from NR⁶, O and S as ring members;
Y is a bond or C1-C3 alkylene optionally substituted with ═O;
Z is an optionally substituted 5-6 membered aromatic ring;
wherein each R is independently H or optionally substituted C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, C1-C8 carboxylic acid, C1-C8 carboalkoxy, carboxamide or C6-C12 heteroarylalkyl;
wherein two R groups on the same nitrogen atom may optionally form a 3 to 8 membered ring, which may be optionally substituted and optionally contain one or two N, O or S as ring members;
wherein R⁶ is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, C6-C12 heteroarylalkyl group, or SO₂R⁷, each of which is optionally substituted with up to four groups selected from R⁷, halo, CN, OR⁷, ═O, C(NR⁷)NR⁷₂, NR⁷₂, COR⁷, COOR⁷, CONR⁷₂, SR⁷, SOR⁷, SO₂R⁷, SO₂NR⁷₂, NR⁷COOR⁷, and COCOOR⁷,
wherein each R⁷ is independently H or C1-C8 alkyl, C2-C8 heteroalkyl, C7-C12 arylalkyl, or diarylalkyl, each of which may be optionally substituted, or two R⁷ groups on the same nitrogen atom may optionally form a 3 to 8 membered ring, which may be optionally substituted and optionally contain up to two heteroatoms selected from N, O and S as ring members; and
with the proviso, wherein
if D is 4-substituted aniline, R³ is H, and Y is a bond, then Z is not thiophenyl;
Z is not a substituted 9-membered bicyclic group comprised of a fused pyrazolyl and pyrimidinyl moiety;
if D is OH or D is NR⁴R⁵, wherein R⁴ is not H, then R³ is not an unsubstituted C1-C4 alkyl;
if Z is 2-(quinolin-5-yl)oxazole and E is C═O, then Y is not a bond; and
if E is ═O and Y is a bond, then Z is not a substituted 9-membered bicyclic ring comprising an imidazole.

2. The compound of claim 1, wherein R³ is H.

3. The compound of claim 1, wherein
Q is selected from phenyl, pyrimidinyl, pyridinyl, pyrazinyl, triazinyl, furanyl, oxadiazolyl, oxazolyl, isoxazolyl, pyrazolyl, thiazolyl, thiophenyl, thiadiazolyl, isothiazolyl, indazolyl, indolyl, morpholinyl and benzimidazolyl,
each of which is optionally substituted with up to four substituents independently selected from the group consisting of CF₃, and optionally substituted C1-C6 alkyl, C1-C6 alkoxy, C3-C10 heterocyclylalkyl, —SO₂R⁸, halo, and −L′NR⁹R⁹, wherein
L′ is a bond or optionally substituted C1-C4 alkylene;
R⁸ is H or C1-C4 alkyl; and
each R⁹ is independently selected from a group consisting of H or C1-C8 alkyl, C1-C8 alkenyl, C2-C8 heteroalkyl, C2-C8 heteroalkenyl, C3-C8 cyclylalkyl, C3-C8 heterocyclylalkyl, C6-C10 aryl, C7-C12 arylalkyl, C4-C12 heteroaryl, C6-C12 heteroarylalkyl, C1-C6 cyanoalkyl, C2-C6 carboxamidoalkyl and —SO₂R⁸, each of which is optionally substituted,
or two R⁹ on the same nitrogen may form an optionally substituted 5-6 membered ring optionally containing O or NR⁸ as a ring member.

4. The compound of claim 1, wherein
Q is selected from phenyl, pyridinyl, pyrazinyl, isoxazolyl, pyrazolyl, thiazolyl, morpholinyl and benzimidazolyl,
each of which is optionally substituted with up to four substituents independently selected from the group consisting of CF₃, C1-C6 alkyl, C1-C6 alkoxy, —SO₂R⁸, halo, C3-C8 heterocyclylalkyl, C1-C6 cyanoalkyl, C2-C6 carboxamidoalkyl, benzyl, phenyl and −L′NR⁹R⁹, wherein
L′ is a bond or optionally substituted C1-C4 alkylene;
R⁸ is H or C1-C4 alkyl; and
each R⁹ is independently selected from H or optionally substituted C1-C8 alkyl, or two R⁹ on the same nitrogen form an optionally substituted 5-6 membered ring optionally containing O or NR⁸ as a ring member.

5. The compound of claim 1, wherein
R⁴ and R⁵ are joined to form a 5-6 membered saturated ring, wherein said ring is piperidinyl, piperazinyl, pyrrolidinyl or morpholinyl, each of which may be optionally substituted with one or more optionally substituted C1-C8 alkyl, benzyl, or phenyl.

6. The compound of claim 1, wherein
m is 2, and
R¹ and R² are optionally connected together to form an optionally substituted 6-membered aromatic group with said ring G.

7. The compound of claim 6, wherein
R¹ and R² are attached to adjacent atoms of said ring G, and
R¹ and R² are joined to form a phenyl ring fused to said ring G wherein the phenyl ring may be optionally substituted with one or more C₁-C₆ alkyl, halo, CF₃, OCF₃, NO₂, NR¹⁰₂, OR¹⁰, SR¹⁰, COR¹⁰, COOR¹⁰, CONR¹⁰₂, NR¹⁰OCR¹⁰ or OOCR¹⁰,
wherein R¹⁰ is H or C₁-C₄ alkyl, or two R¹⁰ attached to the same N may be joined to form an optionally substituted 5-7 membered ring.

8. The compound of claim 1, wherein ring G contains a heteroatom O or NR as a ring member, wherein R is —COOR¹¹ or R¹¹, wherein
R¹¹ is H or C1-C8 alkyl.

9. The compound of claim 1, wherein Z is phenyl, pyridinyl, pyrazinyl, or pyrimidinyl, each of which is optionally substituted with up to four substituents independently selected from the group consisting of —CF₃, —OH, —CN, halo, C3-C8 cycloalkyl, C1-C6 alkoxy and C₁-C₆ alkyl, wherein C3-C8 cycloalkyl, C1-C6 alkoxy, and C1-C6 alkyl are optionally substituted with halo, —OR¹², —CN, —COOR¹² or —CONR¹²₂, wherein each R¹² is independently selected from H and C₁-C₆ alkyl.

10. The compound of claim 9, wherein Z is phenyl or pyridinyl, each of which is optionally substituted with up to four substituents independently selected from the group consisting of —CF₃, —OH, —CN, halo, C3-C8 cycloalkyl, —OR¹¹ and C₁-C₆ alkyl, wherein C3-C8 cycloalkyl, —OR¹¹ and C₁-C₆ alkyl are optionally substituted with halo, —OR¹¹, —CN, —COOR¹¹, —CONR¹¹₂, wherein each R¹¹ is independently selected from H and C₁-C₆ alkyl.

11. The compound of claim 10, wherein phenyl or pyridinyl is substituted with between one and four substituents independently selected from the group consisting of —CF₃, —OH, —CN, t-Bu, cyclopropyl, C2-C4 cyanoalkyl, OR¹¹ and C1-C4 alkyl, wherein C1-C4 alkyl is optionally substituted with —CONR¹¹₂, wherein each R¹¹ is independently selected from H and C₁-C₆ alkyl.

12. The compound of claim 1 wherein E is —C(═O)— or —CH₂—.

13. The compound of claim 1 wherein Y is a bond or —CH₂—.

14. The compound from claim 1, which is selected from

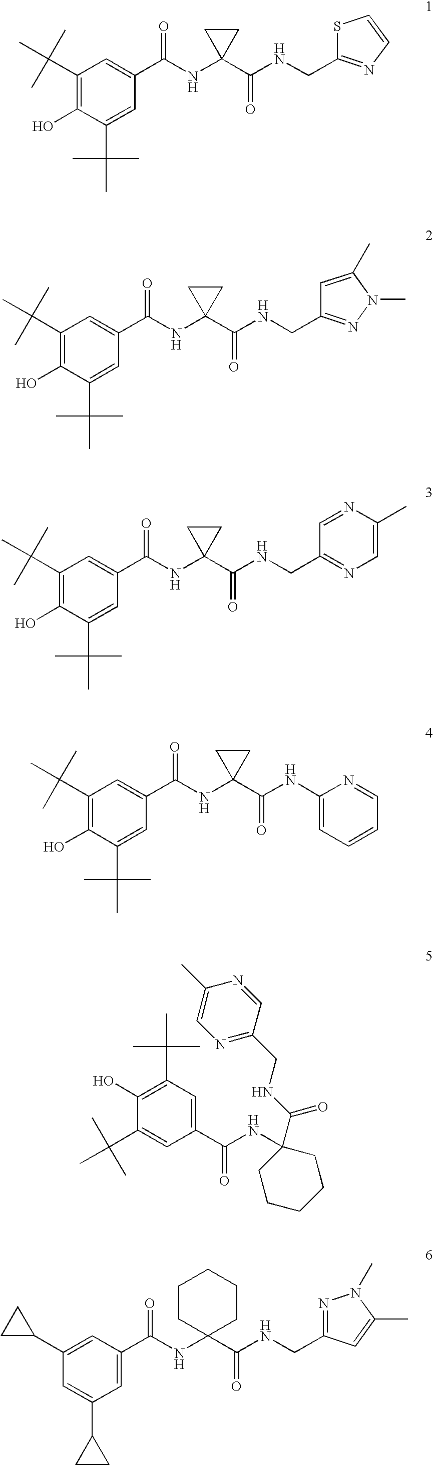

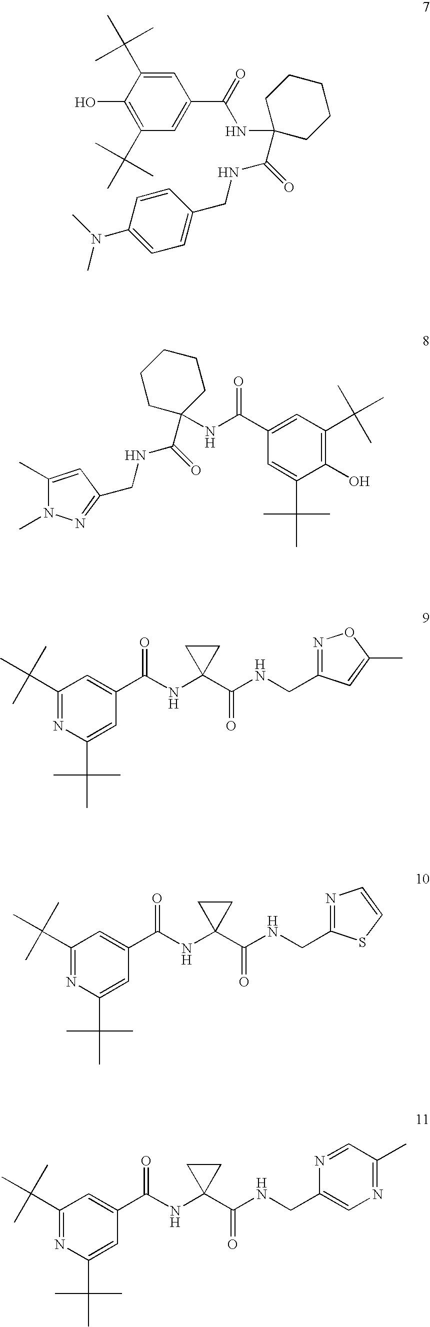

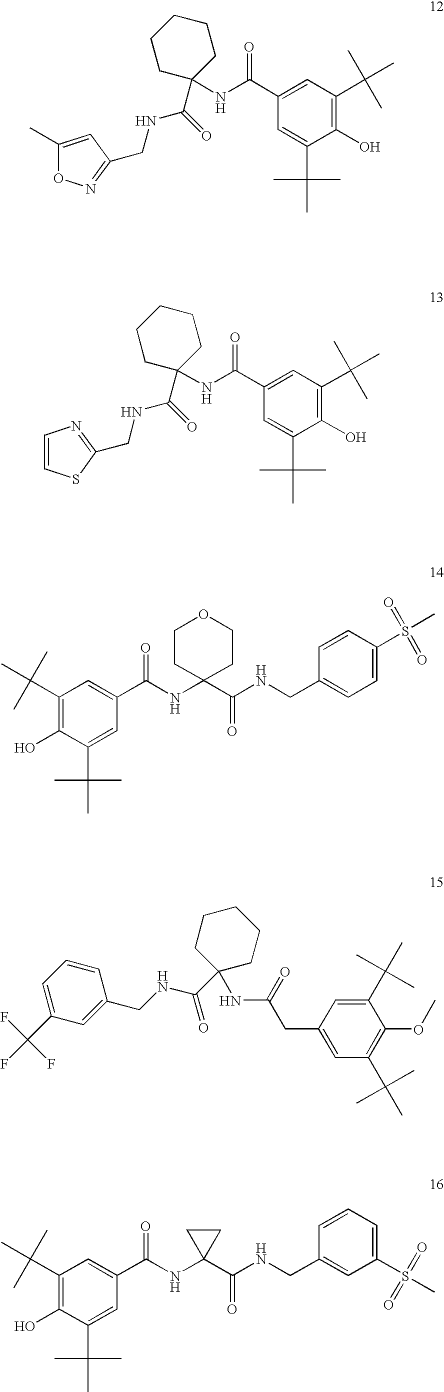

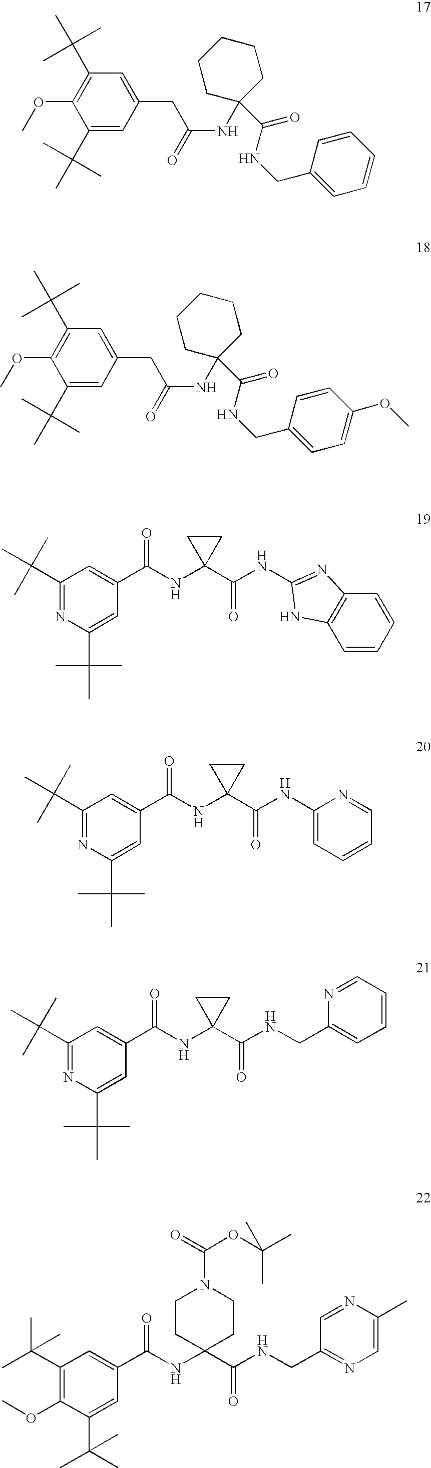

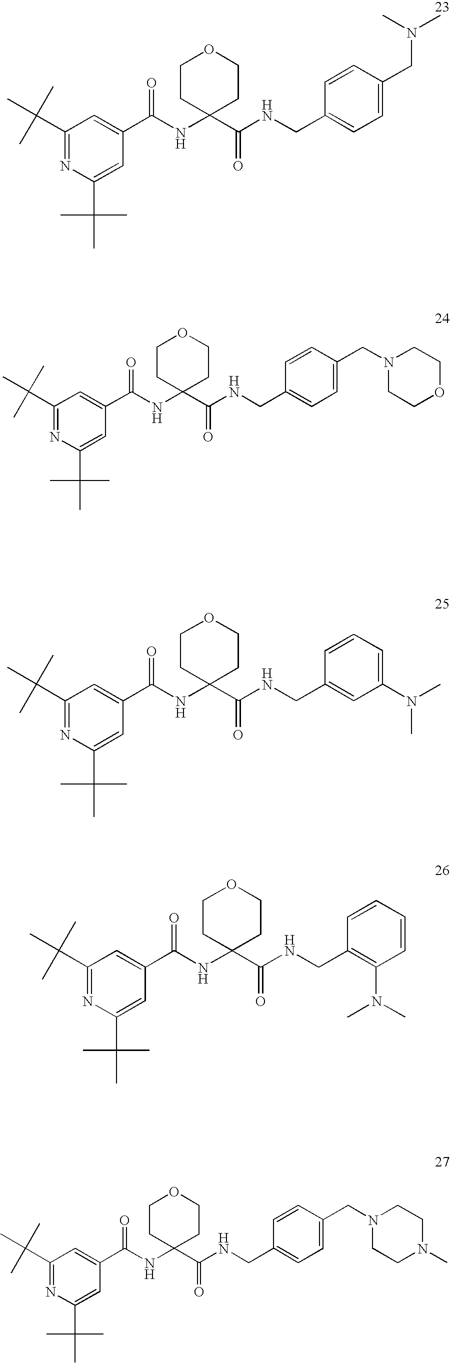

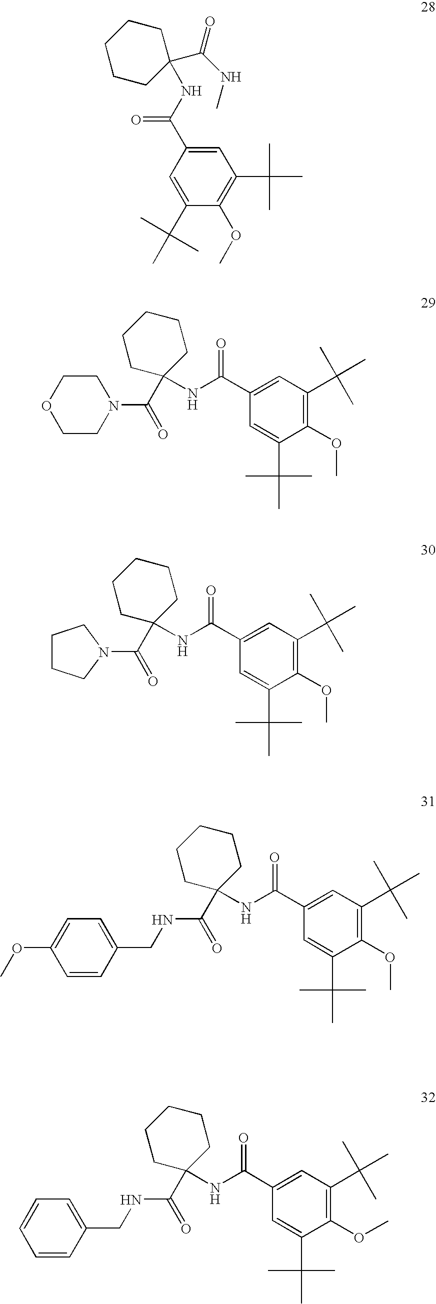

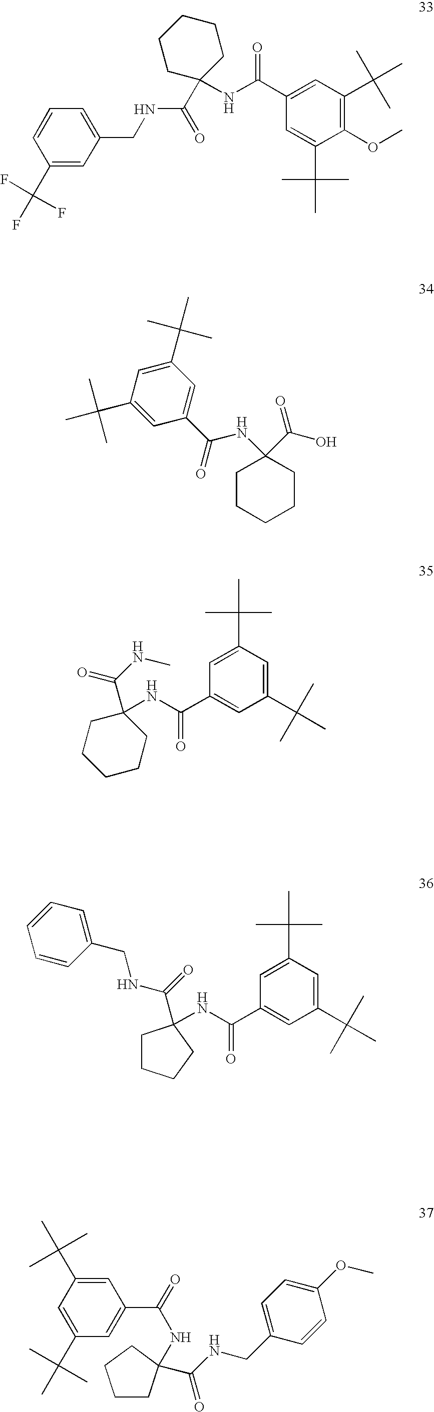

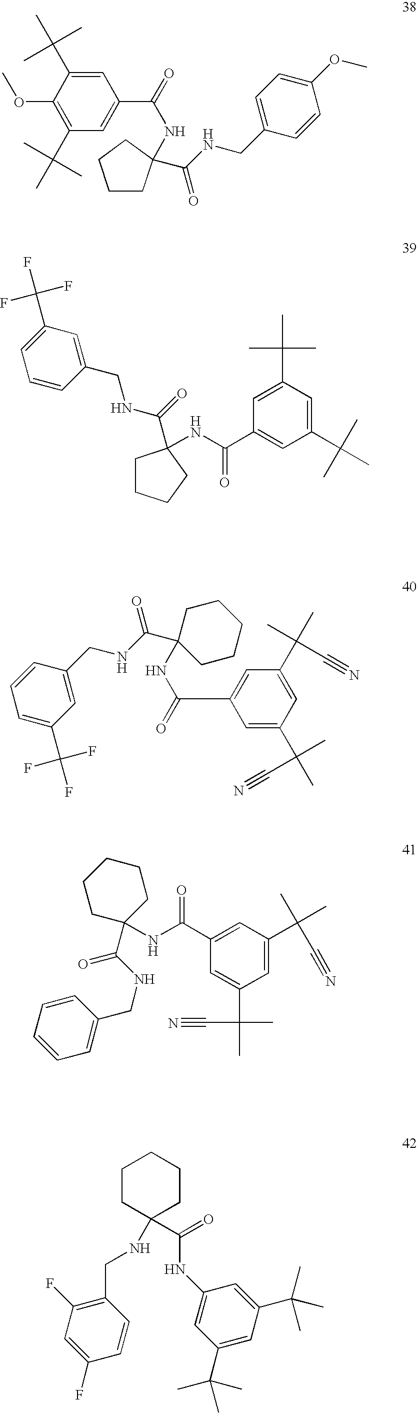

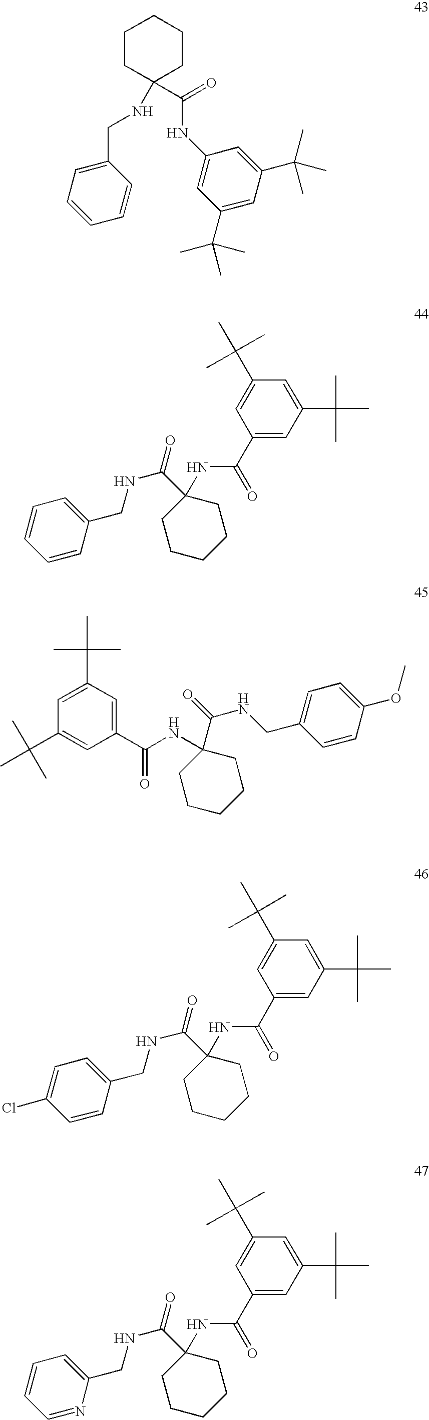

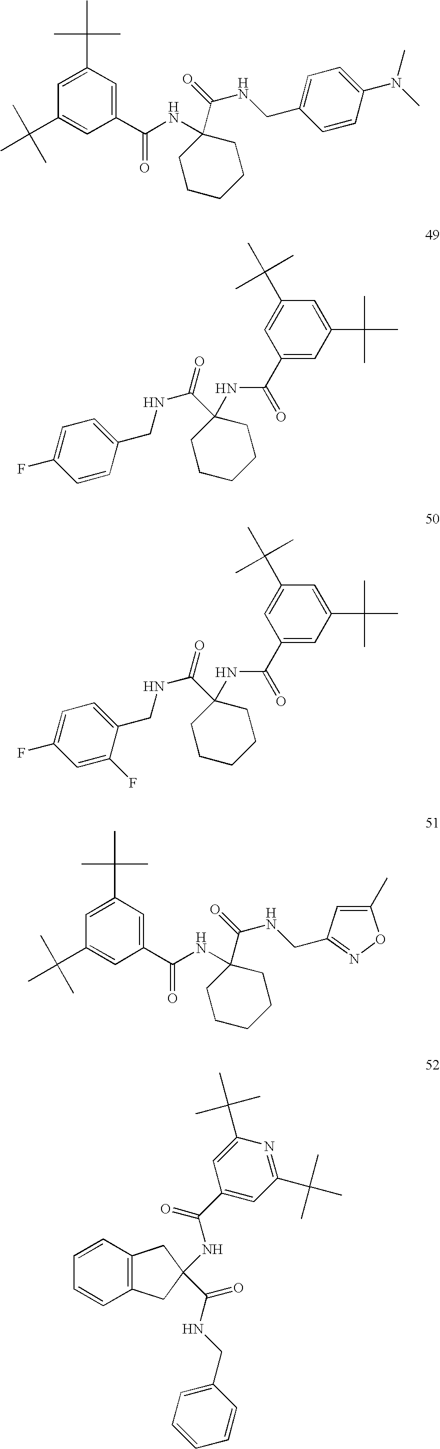

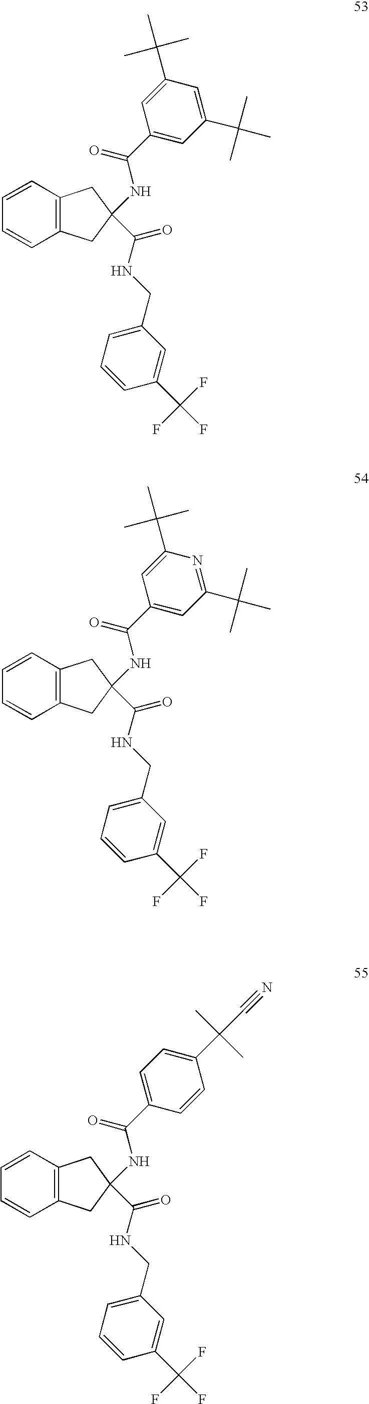

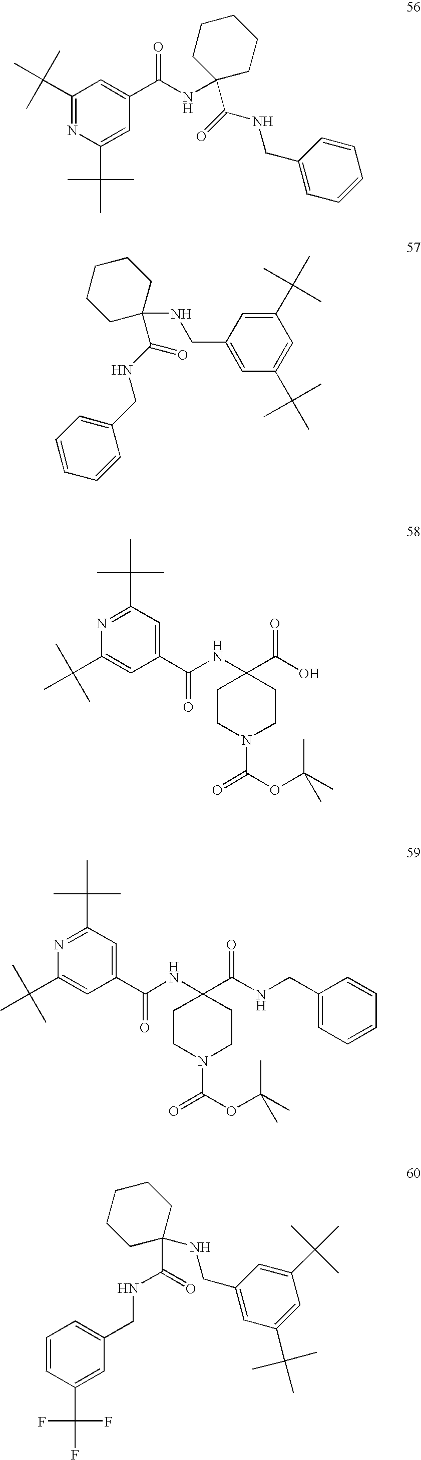

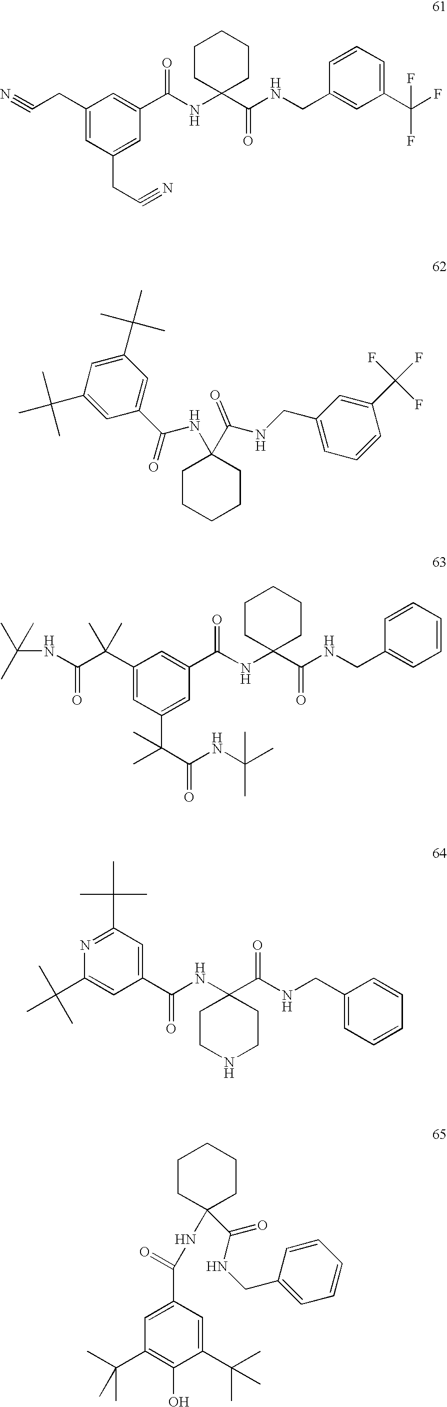

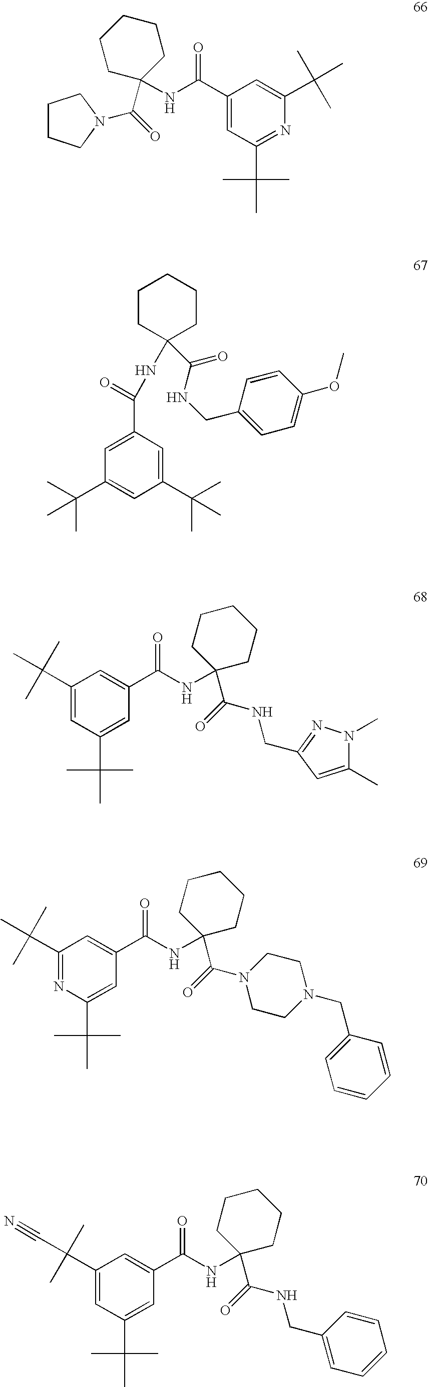

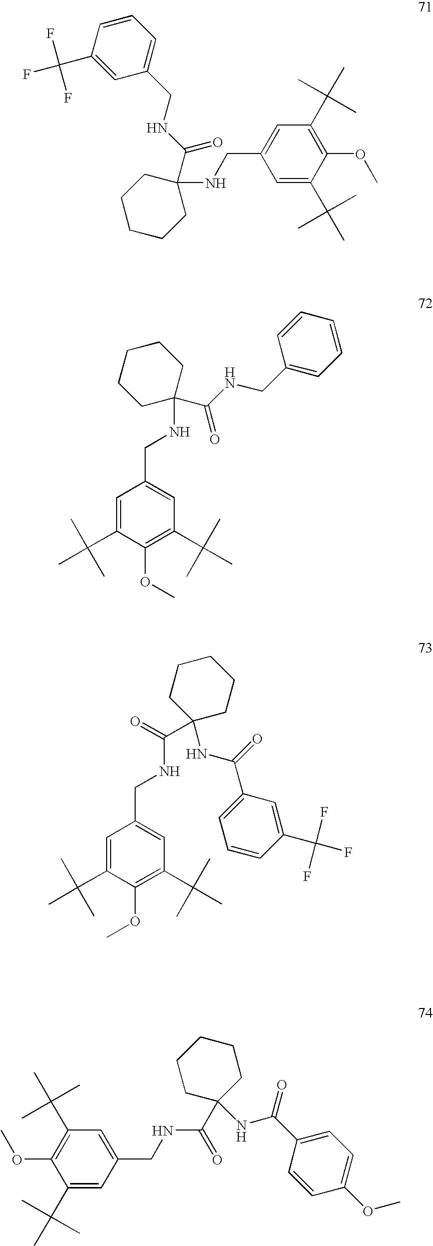

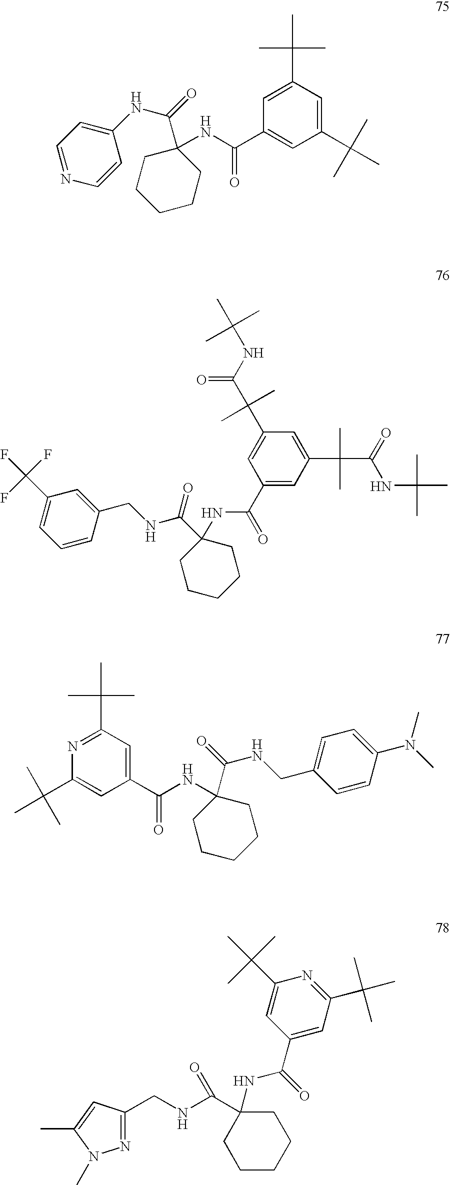

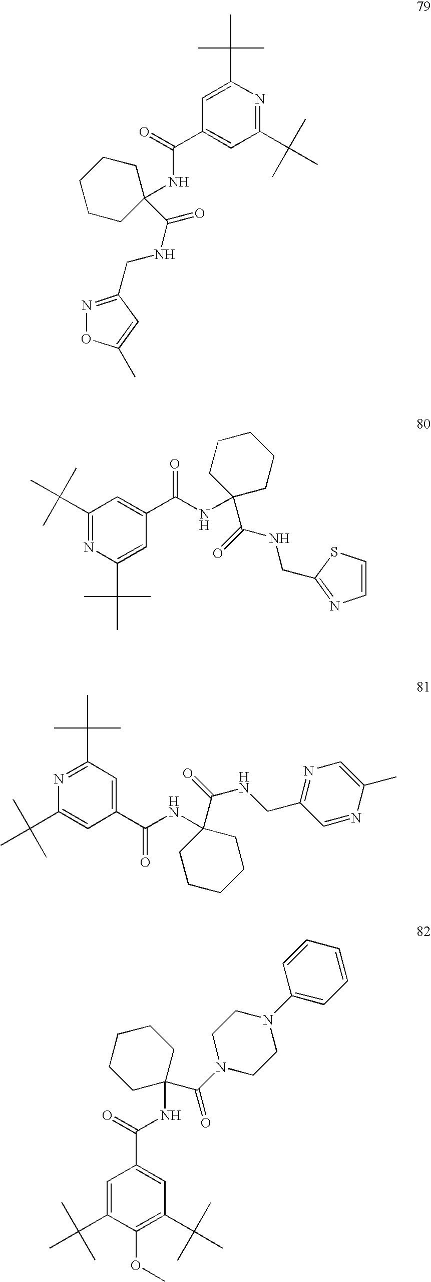

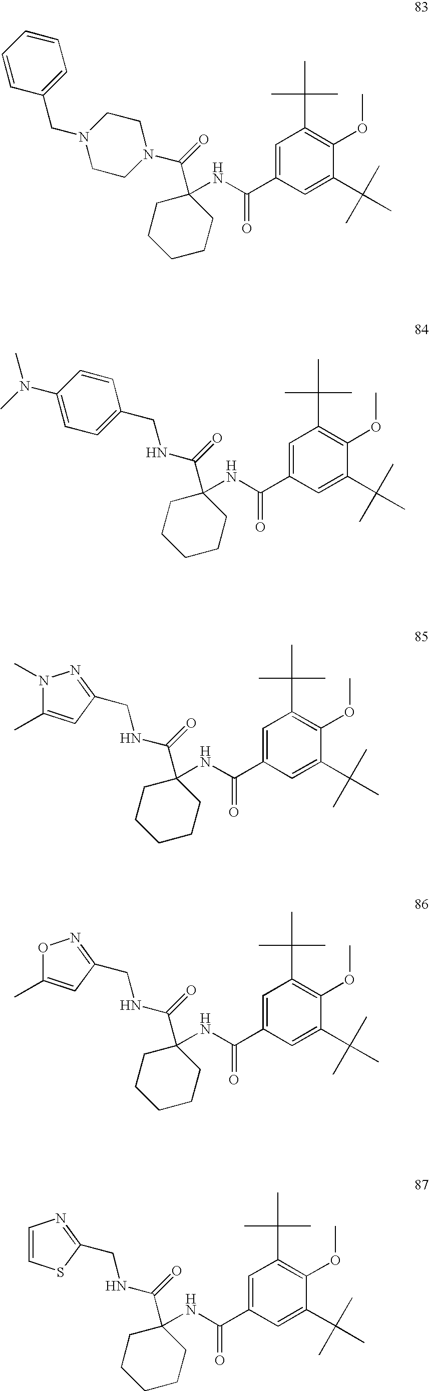

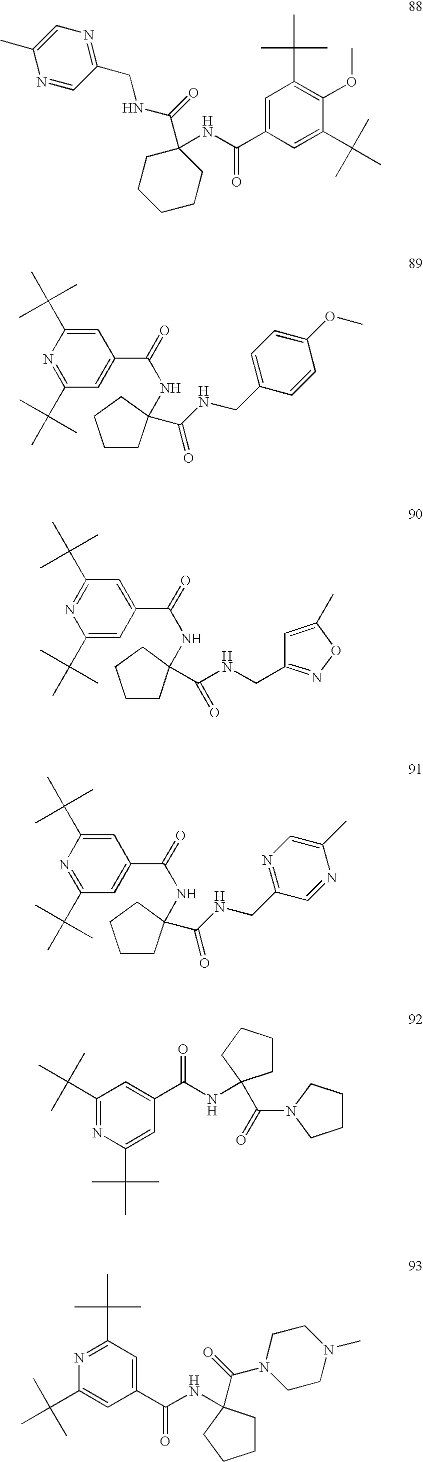

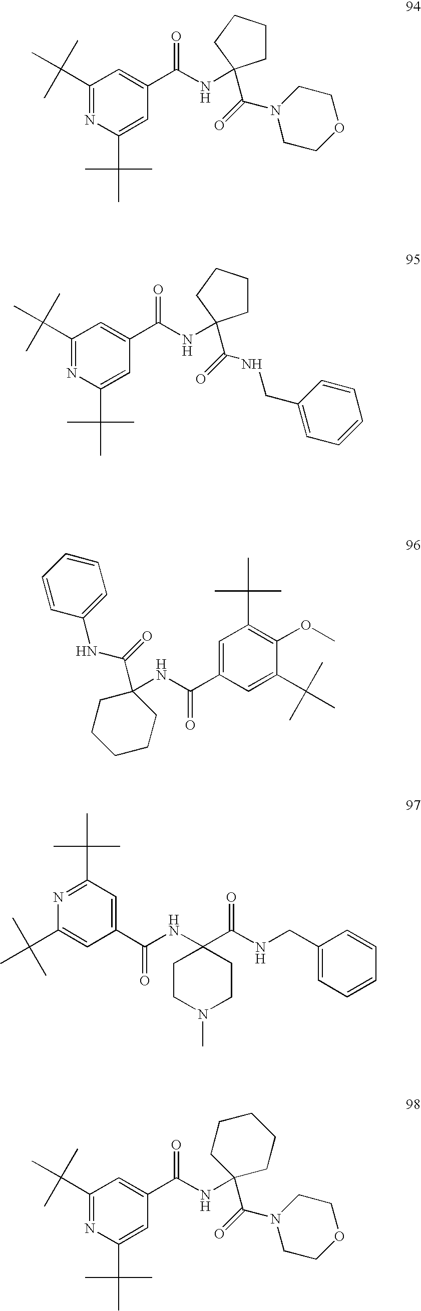

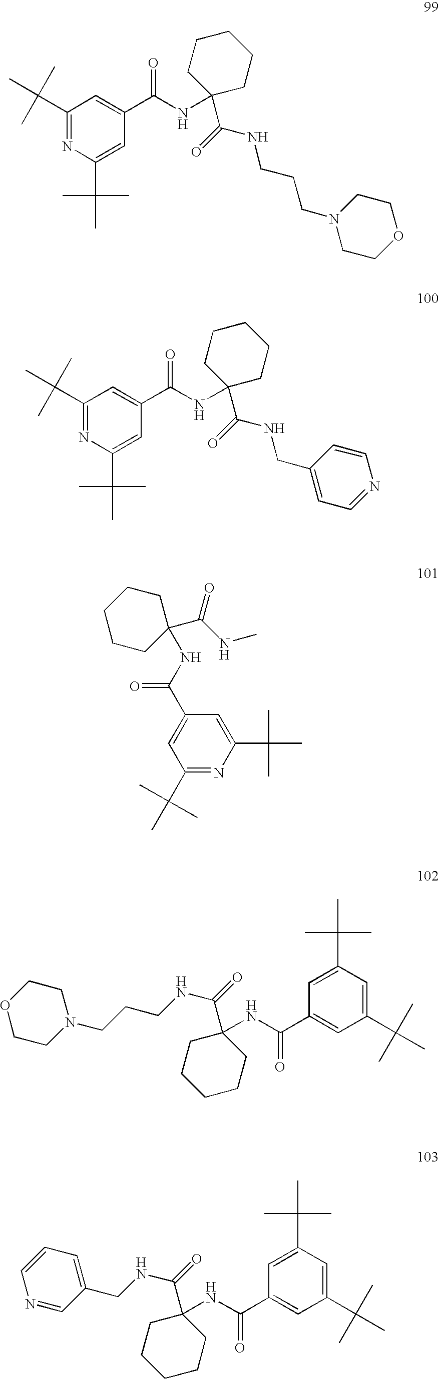

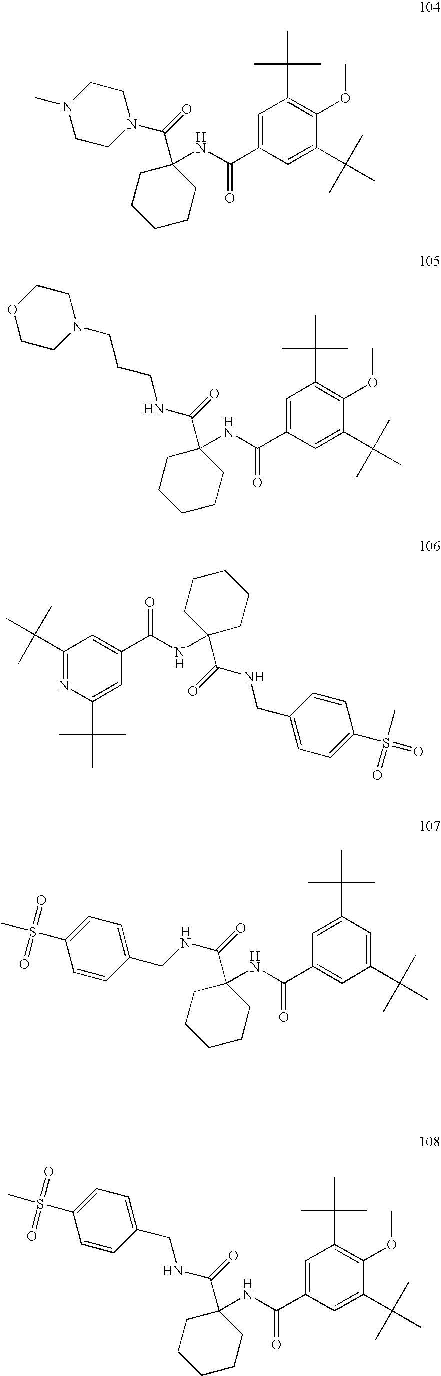

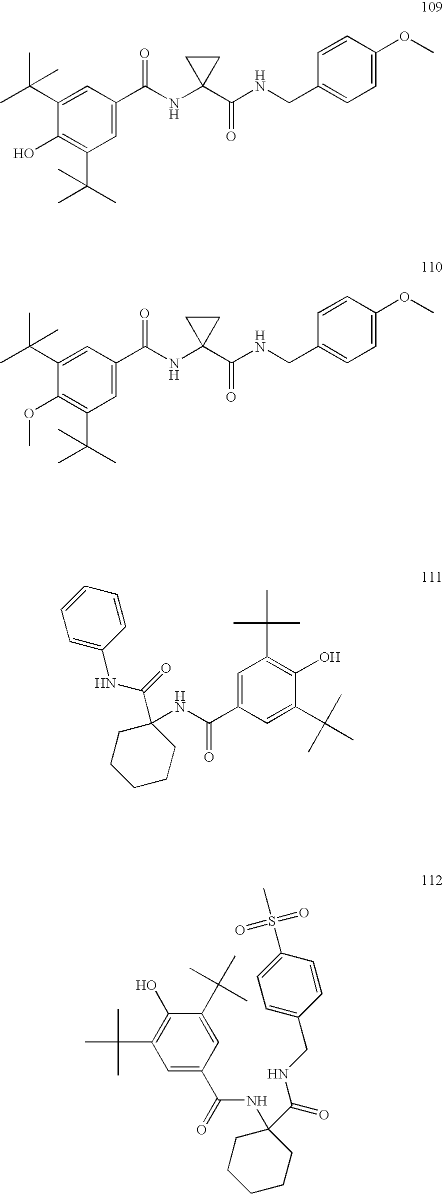

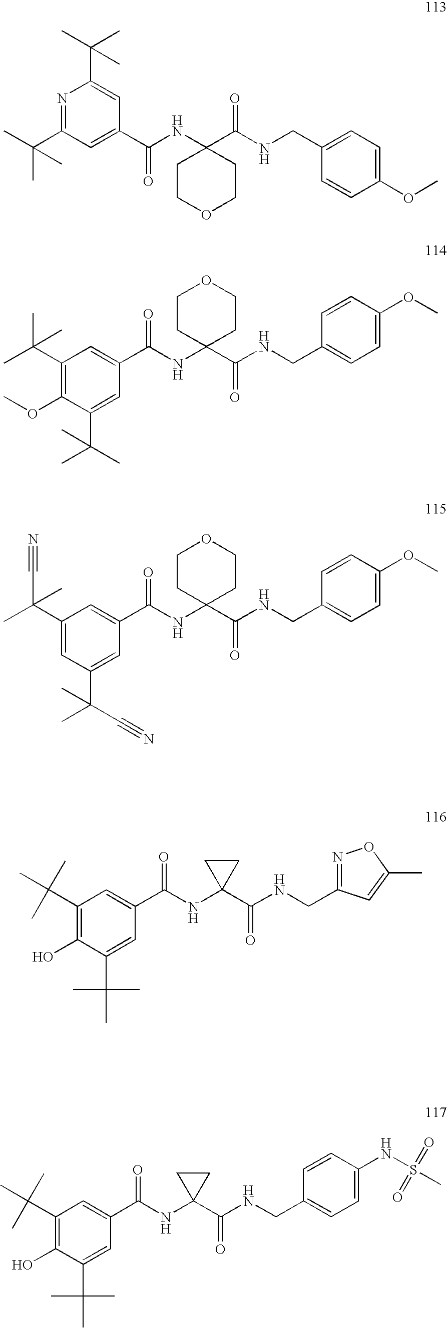

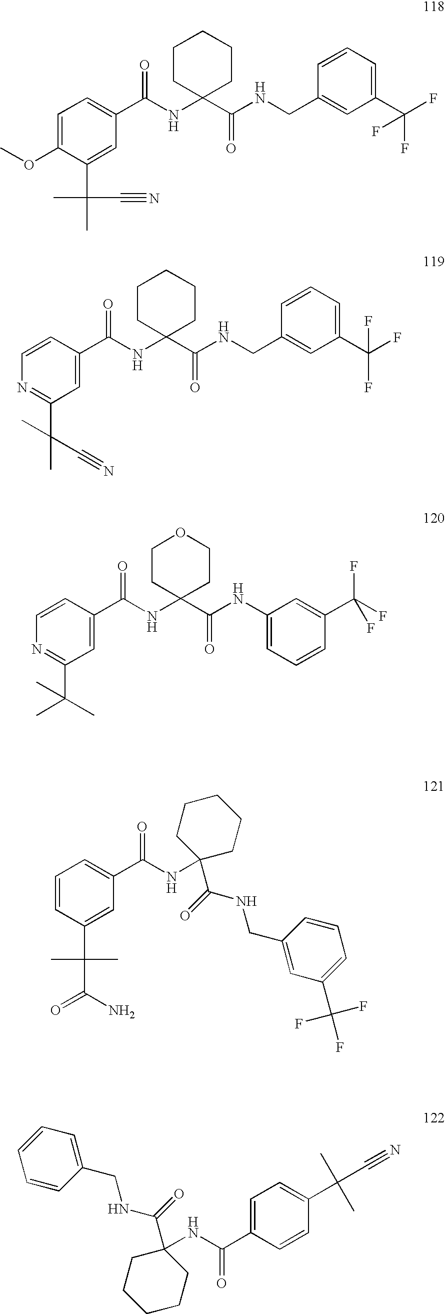

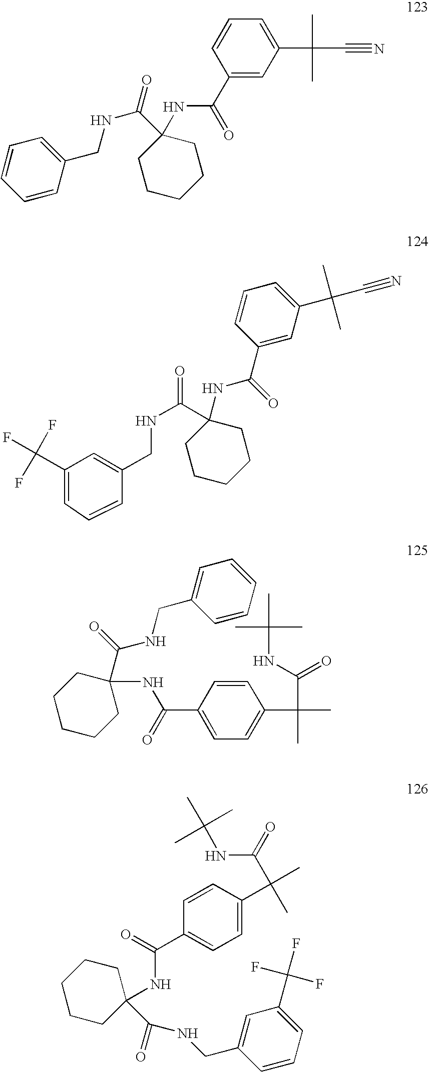

15. A pharmaceutical composition which comprises the compound of claim 1 in admixture with a pharmaceutically acceptable excipient.

16. A method to treat a condition mediated by N-type or T-type calcium ion channels, which method comprises administering to a subject in need of such treatment an amount of the compound of claim 1 or a pharmaceutical composition thereof effective to ameliorate said condition.

17. The method of claim 16, wherein said condition is chronic or acute pain, mood disorders, neurodegenerative disorders, gastrointestinal disorders, genitourinary disorders, neuroprotection, metabolic disorders, cardiovascular disease, epilepsy, diabetes, prostate cancer, sleep disorders, Parkinson's disease, schizophrenia or male birth control.

18. The method of claim 16, wherein said condition is chronic or acute pain.
